SEQUENCE LISTING (111 sequences)

SEQ ID NO: 1 - DNA, 1266 bp, Escherichia coli
  CDS (1)..(1266): uncharacterized protein (B0821)
atggcttctacttttaccagcgacacattgcctgccgatcacaaagcagctatccgtcagatgaagcacgcgctgcgggcgcagcttggcgacgtccagcagatctttaatcagctaagcgatgacattgccacgcgagtggctgaaatcaacgcactcaaagcacagggcgatgccgtctggccggtgctgtcttatgccgatatcaaagcaggtcatgttactgcagagcagcgcgaacagattaaacgtcgcggttgtgcggtgataaaaggccatttcccccgcgaacaagcgctaggctgggatcagtcgatgctggactatctggaccgcaaccgctttgacgaggtctacaaaggccccggcgataatttcttcgggacgctcagcgcttcacgtcccgagatttaccccatctactggtcgcaggcgcaaatgcaggcccgccagagtgaagaaatggcgaatgcgcagtcgtttctcaatcgtctgtggacatttgaaagtgatggaaagcaatggtttaacccggatgtgagcgtcatctaccctgaccgtatccgccgccgtccgcccggaacgacctccaaaggtcttggagcgcataccgactccggggcactggaacgctggctgcttccagcgtatcagcgcgttttcgccaacgtctttaatggcaatctggcgcaatatgatccctggcatgcggcacatcgtacggaagttgaagagtacacggtggacaacaccaccaaatgttccgtgtttcggacattccagggctggacagcgctctctgatatgctgcctggtcaggggctgctgcacgtcgtgcccattcctgaagctatggcgtacgtactgttacgtccgctgcttgatgatgtgccggaggatgaactgtgcggcgtagcgcccggaagagtattgccggtatcagagcaatggcatccactgttgattgaggcgttaaccagcattccaaaactcgaagccggagactccgtctggtggcactgcgacgtcatccattccgttgcccccgttgaaaatcaacaaggttggggcaacgtgatgtacattcctgcggcaccgatgtgcgagaaaaatcttgcctacgcgcacaaggtgaaggccgcactggaaaaaggcgcatcgccgggcgacttcccgcgcgaggactatgaaacaaactgggaaggacgctttacgcttgccgacctcaacattcacggtaagcgagcgttgggcatggatgtttaa

SEQ ID NO: 2 - protein, 421 aa, Escherichia coli
MASTFTSDTLPADHKAAIRQMKHALRAQLGDVQQIFNQLSDDIATRVAEINALKAQGDAVWPVLSYADIKAGHVTAEQREQIKRRGCAVIKGHFPREQALGWDQSMLDYLDRNRFDEVYKGPGDNFFGTLSASRPEIYPIYWSQAQMQARQSEEMANAQSFLNRLWTFESDGKQWFNPDVSVIYPDRIRRRPPGTTSKGLGAHTDSGALERWLLPAYQRVFANVFNGNLAQYDPWHAAHRTEVEEYTVDNTTKCSVFRTFQGWTALSDMLPGQGLLHVVPIPEAMAYVLLRPLLDDVPEDELCGVAPGRVLPVSEQWHPLLIEALTSIPKLEAGDSVWWHCDVIHSVAPVENQQGWGNVMYIPAAPMCEKNLAYAHKVKAALEKGASPGDFPREDYETNWEGRFTLADLNIHGKRALGMDV

SEQ ID NO: 3 - DNA, 525 bp, Escherichia coli
  CDS (1)..(525): uncharacterized protein (B2668)
atggctttgacaaccatttcgccgcatgatgcacaagaattaatcgcacgcggcgcaaagttaatcgatattcgtgatgctgatgaatatcttcgtgaacatattcctgaagcagatctggctccattatccgtgctggaacagtcaggtcttccggctaaattacgtcacgagcaaattatattccactgccaggcaggtaaacgcaccagtaataatgccgataaattagcagcgattgccgcccccgcagaaatctttttactcgaagatgggattgatggctggaaaaaagcaggattgccagtagcggtaaataaatctcaacccttgccgttaatgcgccaggtgcagatcgctgcgggtggtttaatattaatcggcgttgtactgggttataccgtaaatagcggtttcttcttattaagtggctttgttggtgccgggttactgtttgcaggaatcagcggtttttgtggaatggcaaggttgttagataagatgccgtggaaccaacgagcttaa

SEQ ID NO: 4 - protein, 174 aa, Escherichia coli
MALTTISPHDAQELIARGAKLIDIRDADEYLREHIPEADLAPLSVLEQSGLPAKLRHEQIIFHCQAGKRTSNNADKLAAIAAPAEIFLLEDGIDGWKKAGLPVAVNKSQPLPLMRQVQIAAGGLILIGVVLGYTVNSGFFLLSGFVGAGLLFAGISGFCGMARLLDKMPWNQRA

SEQ ID NO: 5 - DNA, 168 bp, Escherichia coli
  CDS (1)..(168): uncharacterized protein (B3362)
atgaagaaactcaccgataagcaaaagtcccgtctctgggagcttcagcgtaatcgtaattttcaggccagtcgccgtcttgaaggcgtcgagatgcctttagtcactcttactgccgcagaggctttagcgcgccttgaagagctgaggagtcactatgagcgataa

SEQ ID NO: 6 - protein, 55 aa, Escherichia coli
MKKLTDKQKSRLWELQRNRNFQASRRLEGVEMPLVTLTAAEALARLEELRSHYER

SEQ ID NO: 7 - DNA, 291 bp, Escherichia coli
  CDS (1)..(291): uncharacterized protein (B3555)
atggaatataaagatccaatgcatgagctgttgagcagcctggaacagattgtttttaaagatgaaacgcagaaaattaccctgacgcacagaacaacgtcctgtaccgaaattgagcagttacgaaaagggacaggattaaaaatcgatgatttcgcccgggttttgggcgtatcagtcgccatggtaaaggaatgggaatccagacgcgtgaagccttcaagtgccgaactaaaattgatgcgtttgattcaagccaacccggcattaagtaagcagttgatggaatag

SEQ ID NO: 8 - protein, 96 aa, Escherichia coli
MEYKDPMHELLSSLEQIVFKDETQKITLTHRTTSCTEIEQLRKGTGLKIDDFARVLGVSVAMVKEWESRRVKPSSAELKLMRLIQANPALSKQLME

SEQ ID NO: 9 - DNA, 387 bp, Synechocystis PCC6811
  CDS (1)..(387): uncharacterized protein (SLL1911)
atggccggattatttggcttatttggtaaaaaggcacagtacgtcgaagatattgaagccaaccccagtcctcagccagaaaaaaaagaggcattttttcttgaaagcgacgatgccaaaagcttgggcaatgccgaatacatgcggactcccatcaaaattaaacggagttttcctaaaactctcaactcccagggaggggaagtggtcaaggagatttctgccatggaggtgaaaaaaatccaggctaatggtcaaccggcccccagtaccaaaatggattccgccccatcccaggccaactctaccccggccaacaacgatcgccgttccaacgacaatagtttggacatgttccgccagatggctaaggatttgaaaaagtaa

SEQ ID NO: 10 - protein, 128 aa, Synechocystis PCC6811
MAGLFGLFGKKAQYVEDIEANPSPQPEKKEAFFLESDDAKSLGNAEYMRTPIKIKRSFPKTLNSQGGEVVKEISAMEVKKIQANGQPAPSTKMDSAPSQANSTPANNDRRSNDNSLDMFRQMAKDLKK

SEQ ID NO: 11 - DNA, 348 bp, Synechocystis PCC6818
  CDS (1)..(348): uncharacterized protein (SLR1062)
atggatctgaagcaggcagaagtcgtcctatgcgagttctatttttctgacctcaagcagaataaactaaggcccgttgttgtttttaaggacaatttgcccttcgatgattttgttggaatgccggtgagtagcagggtaggtcaattaaatgacgatgaaatcgtgttggatgagtcggattttattgagggccttctgtccaagtgctccaaagtgatggtgagaaaaatattcgtcatctcaaaacaggttgtcatcaaaaagcatggtacgctctctacccaaagctttagtaaacttcatctgactttctgtaggtattttggatgtgaaaaccagtcttga

SEQ ID NO: 12 - protein, 115 aa, Synechocystis PCC6818
MDLKQAEVVLCEFYFSDLKQNKLRPVVVFKDNLPFDDFVGMPVSSRVGQLNDDEIVLDESDFIEGLLSKCSKVMVRKIFVISKQVVIKKHGTLSTQSFSKLHLTFCRYFGCENQS

SEQ ID NO: 13 - DNA, 1128 bp, Saccharomyces cerevisiae
  CDS (1)..(1128): undecaprenyl pyrophosphate synthetase (YDL193W)
atgcccacgatgatcaaaaaggatgataaagcaatggagccccctaatgaaaaaccgcatagaaagatcgaaagagatgatgttccagaatcttccaatcacatcccacctccagaatctggtgttttaaagggcggtaaagttaattcaaaaaccagagctttaaaggccgttacaagtatcattgcagacgccgatgagaaccctcagaagaaagtgaacaatgagacgaatggagtccaaaagcaaaagacagaagatttgagtaaaagaataggtaaatttgaataccttttttacaagtttttacttgtgttgttatacatctgcttcgggttgtttcggtacggtcaataccaatataataaaatgaaactaagaatattcagtatcatctacaaccatgcatatacaccacagttgattagacaggacgttattcctctgaaaaaaattcctaaaaggttggccgctatcttggaagtcaagccagttggcgacgttggcggcggtgtgacaggtttattaaatgacgcgagtgaaattgtttgctggactgtttcagctggtataaaacatttgatgttgtacgattacgatggaatattacaaagaaatgttccagagctgagaatggaaattcattccaacctggctaaatattttgggccagctcatgttccaaactacgctgttaaaatacctcattctaacaagatattctacaatctagacggaattgaaaccgagactgatgtaggcaatgagatagaagctaaccaagaaaaggacaaaattgctattgaaatttctttattgtctaacagagatggtagagaaacgattgtcgatctgaccaaaactatggctgagttatgtgcggttaacgaattgagcgtttctgacatcacaatggatttagttgattcagaactgaaacaactagttggacccgaaccagatttactgttatacttcgggccttcgttggatttacaagggttcccaccttggcatattagattaaccgaattttattgggaaaaagataacaacgaagtcatatattcggttttcatccgcggcctaagacagtacgcaggatgtaaagtgaatgttggtaaatga

SEQ ID NO: 14 - protein, 375 aa, Saccharomyces cerevisiae
MPTMIKKDDKAMEPPNEKPHRKIERDDVPESSNHIPPPESGVLKGGKVNSKTRALKAVTSIIADADENPQKKVNNETNGVQKQKTEDLSKRIGKFEYLFYKFLLVLLYICFGLFRYGQYQYNKMKLRIFSIIYNHAYTPQLIRQDVIPLKKIPKRLAAILEVKPVGDVGGGVTGLLNDASEIVCWTVSAGIKHLMLYDYDGILQRNVPELRMEIHSNLAKYFGPAHVPNYAVKIPHSNKIFYNLDGIETETDVGNEIEANQEKDKIAIEISLLSNRDGRETIVDLTKTMAELCAVNELSVSDITMDLVDSELKQLVGPEPDLLLYFGPSLDLQGFPPWHIRLTEFYWEKDNNEVIYSVFIRGLRQYAGCKVNVGK

SEQ ID NO: 15 - DNA, 720 bp, Escherichia coli
  CDS (1)..(720): uncharacterized transcription factor of fatty acid metabolism (B1187)
atggtcattaaggcgcaaagcccggcgggtttcgcggaagagtacattattgaaagtatctggaataaccgcttccctcccgggactattttgcccgcagaacgtgaactttcagaattaattggcgtaacgcgtactacgttacgtgaagtgttacagcgtctggcacgagatggctggttgaccattcaacatggcaagccgacgaaggtgaataatttctgggaaacttccggtttaaatatccttgaaacactggcgcgactggatcacgaaagtgtgccgcagcttattgataatttgctgtcggtgcgtaccaatatttccactatttttattcgcaccgcgtttcgtcagcatcccgataaagcgcaggaagtgctggctaccgctaatgaagtggccgatcacgccgatgcctttgccgagctggattacaacatattccgcggcctggcgtttgcttccggcaacccgatttacggtctgattcttaacgggatgaaagggctgtatacgcgtattggtcgtcactatttcgccaatccggaagcgcgcagtctggcgctgggcttctaccacaaactgtcggcgttgtgcagtgaaggcgcgcacgatcaggtgtacgaaacagtgcgtcgctatgggcatgagagtggcgagatttggcaccggatgcagaaaaatctgccgggtgatttagccattcaggggcgataa

SEQ ID NO: 16 - protein, 239 aa, Escherichia coli
MVIKAQSPAGFAEEYIIESIWNNRFPPGTILPAERELSELIGVTRTTLREVLQRLARDGWLTIQHGKPTKVNNFWETSGLNILETLARLDHESVPQLIDNLLSVRTNISTIFIRTAFRQHPDKAQEVLATANEVADHADAFAELDYNIFRGLAFASGNPIYGLILNGMKGLYTRIGRHYFANPEARSLALGFYHKLSALCSEGAHDQVYETVRRYGHESGEIWHRMQKNLPGDLAIQGR

SEQ ID NO: 17 - DNA, 987 bp, Escherichia coli
  CDS (1)..(987): uncharacterized protein (B2173)
atgaccaggaccaacctcatcaccggttttctcggcagcgggaaaaccacgtcgattcttcatctgttagcccataaagatcccaacgaaaaatgggcggtactggttaatgaatttggggaagtcggaattgatggtgctttgctcgccgatagcggcgcattgctgaaagagatccccggcggctgcatgtgctgcgttaatggtttacccatgcaggtagggttgaataccttactgcgtcagggaaaaccagaccgcttgttgatagagccgaccgggctgggccatccgaaacagatcctcgatctgttaaccgcaccagtctatgaaccgtggatagatctgcgcgccaccttgtgcattctcgatccgcgcttgctgctggacgaaaaaagcgccagcaatgaaaacttccgtgaccagctggctgccgcagacatcattgtcgccaataaatccgaccgtacgacgcccgaaagtgagcaagcgctacagcgttggtggcagcaaaatggtggcgatcgacaattaattcacagtgagcatgggaaagttgacggtcatcttctggatttgccgcgtcgcaatttagccgagttgcccgccagcgccgcgcattctcatcagcatgtcgtgaaaaaagggttagcagcgttaagcctgccagagcatcaacgctggcgtcgcagtctgaacagcgggcaaggatatcaggcctgcggctggatattcgacgctgatacggtattcgacaccattggcattctggaatgggcgcgacttgcaccggtggaacgcgtcaaaggcgtgctgcgtattcccgaagggctggtgcgaatcaaccgtcagggcgatgacctgcacattgaaacgcaaaacgttgcgccaccggacagccgtattgagctgatttccagcagcgaagctgactggaatgccttgcagagcgcgctgttgaagcttcgtttagcgactaccgcgtaa

SEQ ID NO: 18 - protein, 328 aa, Escherichia coli
MTRTNLITGFLGSGKTTSILHLLAHKDPNEKWAVLVNEFGEVGIDGALLADSGALLKEIPGGCMCCVNGLPMQVGLNTLLRQGKPDRLLIEPTGLGHPKQILDLLTAPVYEPWIDLRATLCILDPRLLLDEKSASNENFRDQLAAADIIVANKSDRTTPESEQALQRWWQQNGGDRQLIHSEHGKVDGHLLDLPRRNLAELPASAAHSHQHVVKKGLAALSLPEHQRWRRSLNSGQGYQACGWIFDADTVFDTIGILEWARLAPVERVKGVLRIPEGLVRINRQGDDLHIETQNVAPPDSRIELISSSEADWNALQSALLKLRLATTA

SEQ ID NO: 19 - DNA, 1155 bp, Glycine max
  CDS (1)..(1155): uncharacterized protein (GM50181105)
atggagcacgacgaagacgaagagccacctcttgctgttcagattcaagggaatgatgaatccgtttctcagcaatcttcttctgttggggtcactctcatcaccggttatcttggttcgggcaagtccactctagtgaatcatattttgaattcgcaacatgggaagaggattgctgtcattttaaatgagtttggtgaggaaattggcgtcgaaagagcgatgatcaatgaaggagataagggtgcattggttgaagaatgggttgagcttgccaatgggtgtatatgttgcacagtcaagcatagtttggttcaagcacttgaacaacttgttcagagaaaggaaaggcttgaccatatattgctggaaaccactggattggcaaatccagctcctttggcatctgttctatggttggatgaacagttggaatcagaagtgaagcttgattctattgtcacggtggtggatgctaaaaatttgcgcttccagcttgatgagcatcgtggatcatcttcatttcctgaagcatattttcagatagcatttgcggacattataattcttaacaaggttgatttggtatctgtagagagctctggagctctggaggaacttgaggtggaaatacataacattaattctcttgcagagataatacattctgttcgatgtcaagttgacttgtctaagatattgaaccgccaagcttatgataccgcacgtgccacacaattagaggcattgttagaagaaagtcgttctttgtctaccaaaaagcttcatgatagtcttcatgatagtgacgtgagaaccatatgcatttgtgagacacggatgattaatcttgataagactcggatatggcttgaggagattctctgggagaagaaatatgatatggatgtataccgttgcaaaggagtgttaagtgttcaaaattctgatcaacttcatactttgcaggcagtgaaggaactgtatgagattgttccatctcgcaagtgggaaaaggaagagaaacgcataaataagatagtgtttattggtaatttgatctacttttctagttttaacacaattttgagtgaaaaaggccttcttcagcttgttaacattctttcctttttctttctgaatgacaggtcataa

SEQ ID NO: 20 - protein, 384 aa, Glycine max
MEHDEDEEPPLAVQIQGNDESVSQQSSSVGVTLITGYLGSGKSTLVNHILNSQHGKRIAVILNEFGEEIGVERAMINEGDKGALVEEWVELANGCICCTVKHSLVQALEQLVQRKERLDHILLETTGLANPAPLASVLWLDEQLESEVKLDSIVTVVDAKNLRFQLDEHRGSSSFPEAYFQIAFADIIILNKVDLVSVESSGALEELEVEIHNINSLAEIIHSVRCQVDLSKILNRQAYDTARATQLEALLEESRSLSTKKLHDSLHDSDVRTICICETRMINLDKTRIWLEEILWEKKYDMDVYRCKGVLSVQNSDQLHTLQAVKELYEIVPSRKWEKEEKRINKIVFIGNLIYFSSFNTILSEKGLLQLVNILSFFFLNDRS

SEQ ID NO: 21 - DNA, 450 bp, Escherichia coli
  CDS (1)..(450): uncharacterized membrane protein (B2670)
atgttctcaccgcagtcacgcttgcgtcatgcagttgcagatacgttcgcgatggttgtttactgttctgtcgtgaacatgtgtattgaagttttcctctccggaatgagcttcgaacagtctttttattccagattggtagcgattccggtgaacatcttaattgcatggccatacggtatgtaccgtgatctgtttatgcgcgcggcacgcaaagttagcccgtcgggctggataaaaaatctggcggatatcctggcttatgtgacgttccagtcaccggtgtatgtggcgatcttgttagtggtgggcgcagactggcatcagattatggcggcggtcagttcaaacatcgttgtttcgatgttgatgggggcggtttatggctacttcctcgattattgccgccgactgtttaaagtcagccgttaccagcaggtaaaagcctaa

SEQ ID NO: 22 - protein, 149 aa, Escherichia coli
MFSPQSRLRHAVADTFAMVVYCSVVNMCIEVFLSGMSFEQSFYSRLVAIPVNILIAWPYGMYRDLFMRAARKVSPSGWIKNLADILAYVTFQSPVYVAILLVVGADWHQIMAAVSSNIVVSMLMGAVYGYFLDYCRRLFKVSRYQQVKA

SEQ ID NO: 23 - DNA, 1632 bp, Saccharomyces cerevisiae
  CDS (1)..(1632): peroxisomal-coenzyme A synthetase (YBR222C)
atgacaagtgccgctactgttactgcttcgttcaacgatacttttagcgtatccgataatgtcgccgttattgttcctgaaacggacactcaggtgacctacagggatctatcccacatggtgggtcacttccagaccatgttcacaaatcctaattctccattgtacggagctgttttcagacaagatacagtggcgatatccatgcgtaatgggctggaatttatcgtcgctttcctcggtgctactatggacgctaaaattggcgcgcccttgaatcccaattataaggaaaaggagttcaatttttatttgaatgacctgaaatctaaggcgatttgcgtcccaaagggtaccacaaagttacagagttctgaaattctaaaatctgcctccacgtttggatgttttatcgtagagctggccttcgatgcgaccaggtttagggtagagtatgatatatactctccagaggacaactacaaaagggttatttaccggtctttgaacaacgccaaatttgtcaacacaaatcccgttaaattccctgggtttgcccgttccagtgacgttgccctgattttgcataccagtggtaccacctccactccaaaaacggtgcctttgttacatttgaacattgtgagaagcacgttgaacattgctaacacttacaagctaacgcccttggacagatcttatgtcgtgatgcctcttttccacgtccatgggttaattggtgttttactttccacttttagaactcagggttctgttgtggttcccgatggattccatccaaagttattctgggaccaatttgttaagtacaactgtaattggttcagttgcgttcccacaataagcatgattatgctgaacatgcccaaaccaaaccctttcccacacattagattcatcagatcgtgttcttctgctttggctccagcaacgttccataagctggagaaggaattcaatgcacctgtcttggaggcctatgcgatgaccgaagcatcacatcaaatgacctcaaacaatctgcctccaggaaagagaaagcctggtactgtgggccagccacaaggagtcaccgtcgtcattctagatgacaatgacaatgtcttgcccccgggcaaagtcggcgaagtttccatcagaggcgaaaacgtcactttggggtatgctaataatccaaaagctaacaaggagaacttcaccaagagagagaactatttcagaaccggtgaccaaggttatttcgaccctgaggggtttttggtccttacaggcagaatcaaagagcttatcaacaggggtggtgaaaagatttcacccattgagctcgacggcattatgctatcgcatccaaagatcgatgaagccgttgcatttggtgttcccgacgatatgtacggccaagtagttcaagccgccattgttttgaagaagggagaaaaaatgacctacgaagaactggtgaacttcttaaagaagcacctagcctctttcaaaattccaaccaaggtgtactttgttgataagctaccaaaaaccgctacaggtaaaatccagagaagagttatcgcagaaacttttgctaagagcagcagaaataagagtaagttgtag

SEQ ID NO: 24 - protein, 543 aa, Saccharomyces cerevisiae
MTSAATVTASFNDTFSVSDNVAVIVPETDTQVTYRDLSHMVGHFQTMFTNPNSPLYGAVFRQDTVAISMRNGLEFIVAFLGATMDAKIGAPLNPNYKEKEFNFYLNDLKSKAICVPKGTTKLQSSEILKSASTFGCFIVELAFDATRFRVEYDIYSPEDNYKRVIYRSLNNAKFVNTNPVKFPGFARSSDVALILHTSGTTSTPKTVPLLHLNIVRSTLNIANTYKLTPLDRSYVVMPLFHVHGLIGVLLSTFRTQGSVVVPDGFHPKLFWDQFVKYNCNWFSCVPTISMIMLNMPKPNPFPHIRFIRSCSSALAPATFHKLEKEFNAPVLEAYAMTEASHQMTSNNLPPGKRKPGTVGQPQGVTVVILDDNDNVLPPGKVGEVSIRGENVTLGYANNPKANKENFTKRENYFRTGDQGYFDPEGFLVLTGRIKELINRGGEKISPIELDGIMLSHPKIDEAVAFGVPDDMYGQVVQAAIVLKKGEKMTYEELVNFLKKHLASFKIPTKVYFVDKLPKTATGKIQRRVIAETFAKSSRNKSKL

SEQ ID NO: 25 - DNA, 1710 bp, Brassica napus
  CDS (1)..(1710): peroxisomal-coenzyme A synthetase (BN51408632)
atggcggcgactaagtctcgtgacatcgacgaccttccgaaaattcaggcgaactacaccgcgttgacgccgctctggttcttagacagggctgcggcggttcatccgacgaggaaatcgctgattcacggatccttggagtacacgtggcggcagacttacgagcgatgtcgccgtctagcctccgctctcgccgatcgttcgattggacctggctccacggtggctgtaattgcacccaacactccagcaatgtacgaagctcatttcggaataccaatgtgtggagccgtcttgaacgccgtcaacatccgtctcaacgcccccactatcgctttccttctcggccactctcagagcgctgttatcatggtggatcaagagtttttcactcttgcagaggagtctttgagactcatggaggagaaagctgggagcagcttcaaacgcccgctcctaatcgtcataggtgatcacacttgtcctccagagtcgcttcaccgggctttgtcgaaaggagttgtagaatacgaggattttcttggaagtggagatcctaactatgcgtgggagacaccagctgatgagtggcagagtattgctcttggttacacctcgggaacaaccgctagcccgaaaggtgtggtgcttcatcatcgaggagcgtatctaatggctttaagcaatcctcttatttgggggatgcaagaaggctctgtttacttgtggactctccctatgtttcattgcaatggttggtgtttcacttgggctcttgctgcgctctccggtactaacatctgtctccgtcaggtcacggcgaaagaagtgtattcgagcatagccaagtataacgttacccatttctgtgcggctcctgtggtcctcaacactattgtcaatgctcctcaagaggacactatcctcccccttccccatactgtccatgtcatgaccgcaggagctgctcctccaccttctgttctcttctccatgaaccagaagggcttccgagtcactcacacctatgggctatccgagacgtacggtccttccaccgtagccgcctggaagcccgagtgggactccctccctcctgagacgcaggccaagctcaatgctcgccaaggtgtccgctacatcggcatggagcagcttgatgtcatcgacactcagacaggaaaacctgttcctgcagacggtaaaaccgccggagagattgttttccgagggaacatggtgatgaaaggatacttaaagaatcctaaagctaacgaggagactttcgctggtgggtggttccattcaggggatatcgcggtgaaacatccagacaactacatcgagatcaaggacaggtcgaaggacattataatctccggcggtgagaacatcagcagcgtggaggtggaaaacgtcgtgtatcatcacccggcggtgcttgaagcctctgttgtggccaggccagacgagcggtggcaggaatctccgtgtgcttttgtgacggtcaagagcggttacgagaaacaagaccagaataatttggctcaggatataatgaaattctgcaaggagaagctgccggcgtactgggttccgaagtcggtggtgtttgggccgttaccgaagactgctactggaaagattcagaagcatgttttgaggactaaggcgaaagagttaggaccagtaccaagaagcaggttgtga

SEQ ID NO: 26 - protein, 569 aa, Brassica napus
MAATKSRDIDDLPKIQANYTALTPLWFLDRAAAVHPTRKSLIHGSLEYTWRQTYERCRRLASALADRSIGPGSTVAVIAPNTPAMYEAHFGIPMCGAVLNAVNIRLNAPTIAFLLGHSQSAVIMVDQEFFTLAEESLRLMEEKAGSSFKRPLLIVIGDHTCPPESLHRALSKGVVEYEDFLGSGDPNYAWETPADEWQSIALGYTSGTTASPKGVVLHHRGAYLMALSNPLIWGMQEGSVYLWTLPMFHCNGWCFTWALAALSGTNICLRQVTAKEVYSSIAKYNVTHFCAAPVVLNTIVNAPQEDTILPLPHTVHVMTAGAAPPPSVLFSMNQKGFRVTHTYGLSETYGPSTVAAWKPEWDSLPPETQAKLNARQGVRYIGMEQLDVIDTQTGKPVPADGKTAGEIVFRGNMVMKGYLKNPKANEETFAGGWFHSGDIAVKHPDNYIEIKDRSKDIIISGGENISSVEVENVVYHHPAVLEASVVARPDERWQESPCAFVTVKSGYEKQDQNNLAQDIMKFCKEKLPAYWVPKSVVFGPLPKTATGKIQKHVLRTKAKELGPVPRSRL

SEQ ID NO: 27 - DNA, 1698 bp, Brassica napus
  CDS (1)..(1698): peroxisomal-coenzyme A synthetase (BN51423788)
atgataactgcaactctacaagaacctcagattcatcaaccggtggatacaactactccccccgccgatgctcctcctactccgccgcgtattttccgatcaaagcttccggacatagacattcccaaccacctccctctccacacctactgcttccagaagctctcctctgtttccgacaagccttgtctgatcgtagggtccacgggaaaaaactacacgtacggcgaaacgcacctcatatgtcggagagtcgccgccgggctacacaaaatggggattcgaaaaggcgacgtgataatgatcctccttcaaaactcagccgagttcgtcttctccttcatgggcgcttccatgatcggcgccgtctccaccaccgcgaaccctttctacacttctcaggagattcacaaacaggtcaaatcctccggagctaagcttataatcactcactcccattacgtcgataagctgagaaacctcgacggagaaacaagaatcggtgaagatctcaccgttatcaccacggaggagaatcctcccccggagaattgtcttcctttctccacactactactcactgacgacgagacaacctcgctagatgttgttgatgtcggtggtgatgacgccgcggcgcttcctttctcctctggcacgacggggttacctaaaggagtagttttaacgcacaagagcttaatcacgagcgtcgcgcaacaagtcgatggagacaacccgaatctttacctgaaaccaaacgacgtcgtactctgcgttttgcctcttttccacatctactcgctcaacagcgttcttctcaactccatccgatccggtgcgacggttcttttgatgcacaaattcgaaatcggggcgttattggatctaatacagagacacaaggtgacggtagcggcgcttgtcccgccgcttgtgattgctttggccaagaacccaactgttaactcttacgatctctcttccgttagattggttctctctggtgcagctcccttaggcaaagatctcgaggatagtctaggccgccgtctccctcaggccgtccttggccagggatatggtatgacagaggcaggaccagtgttgtcaatgagccttgggtttgctaaagagccaactccgtcaaaatcaggctcttgtgggactgtagtccgaaacgcagagcttaaagtggttcaccttgagacacgtctctctcttggttacaaccaacctggtgagatttgtatccgcggtcaacaaatcatgaaagagtacttgaacgatccggaagccacgtcagctacaattgacgaggaaggttggcttcacacaggggacattgggtatgttgatgaagctgatgagatattcattgttgatcgactcaaagaagtcatcaagttcaaaggctttcaggtgcctccagctgagctagaggctttgctcatcaatcaccactccattgcggatgctgcggttgttcctcaaagagatgaagtggctggagaagttccggtggctttcgtagtccgatcaaatggaaacgttatcacggaagaagatataaaagaatatatagccaaacaggtggtattctacaagagattgcacaaggtcttctttgttccctccattcccaaatctccttccggaaaaattctgagaaaggatcttaaagccaaactttgttga

SEQ ID NO: 28 - protein, 565 aa, Brassica napus
MITATLQEPQIHQPVDTTTPPADAPPTPPRIFRSKLPDIDIPNHLPLHTYCFQKLSSVSDKPCLIVGSTGKNYTYGETHLICRRVAAGLHKMGIRKGDVIMILLQNSAEFVFSFMGASMIGAVSTTANPFYTSQEIHKQVKSSGAKLIITHSHYVDKLRNLDGETRIGEDLTVITTEENPPPENCLPFSTLLLTDDETTSLDVVDVGGDDAAALPFSSGTTGLPKGVVLTHKSLITSVAQQVDGDNPNLYLKPNDVVLCVLPLFHIYSLNSVLLNSIRSGATVLLMHKFEIGALLDLIQRHKVTVAALVPPLVIALAKNPTVNSYDLSSVRLVLSGAAPLGKDLEDSLGRRLPQAVLGQGYGMTEAGPVLSMSLGFAKEPTPSKSGSCGTVVRNAELKVVHLETRLSLGYNQPGEICIRGQQIMKEYLNDPEATSATIDEEGWLHTGDIGYVDEADEIFIVDRLKEVIKFKGFQVPPAELEALLINHHSIADAAVVPQRDEVAGEVPVAFVVRSNGNVITEEDIKEYIAKQVVFYKRLHKVFFVPSIPKSPSGKILRKDLKAKLC

SEQ ID NO: 29 - DNA, 1656 bp, Brassica napus
  CDS (1)..(1656): peroxisomal-coenzyme A synthetase (BN51486050)
atggctccacaagaagacgccatgcagaaacagagcagcaacaagagtgacgtcatattccgatcaaagcttccggatatttacatcccgaaccacctccctctccacgactacatcttccaaaacatctccgagttcgcctccaagccttgcctgatcaacggtcccaccggccacgtgtacacttactccgaggtccatgtcgcttcccgtcgcatcgccgccggttttcaaaaactcggcgttaaccaaaacgacgtcgtgatgatcctcctctcgaattgccccgagttcgtcctctctttcctcgccgcctccttccgcggcgcaacggccaccgccgctaacccgtttttcactccggcggagatcgccaaacaggcgaaagcctcgaactcgaagctcatcgtcaccgagtctcgctacgtcgataaaatcaaagacctccaaaacgacggcgttataatcgtctgcaccgacgaggaaccttctccgatcccggaaggctgcctccgattcaccgagttgactcagtcaaccgaaatggaaacggtggagatttcttccgacgacgtggtggcgcttccttactcctccggcacgacgggtctacccaaaggagtgatgctgactcacaagggactagtcacgagcgtcgctcagcaagtcgacggcgataatccgaatctttacttccacagcgatgacgtcatactctgtgtcttgcccttgttccatatctacgctttgaactcgatcatgctgtgtgggcttagagttggtgcctccatcttgatcatgcccaagttcgagattaatctgctcttggagctcatacagcggtgtaaagtcacggttgctccgatggttccgccgattgttttggccatggcgaagtcgccggagacggagaagtatgacttgagctcgattagggttgtcaagtctggcgctgccccgcttggtaaggagcttgaagatgccgtcagtgccaagtttcctaacgccaaactcggtcagggatacggaatgacggaagcaggtccggtgctagcaatgtcgttagggttcgcgaaagagccgtttccagtgaagtcgggagcttgtggtacggtggttagaaacgccgaaatgaaaatcatcgatccagacaccggagactcactttccaaaaacaaacctggagagatttgcatccgtggtcaccagatcatgaaaggttacctcaacaacccggcggctacagcagagaccatagacaaagacggctggcttcacaccggagatatcgggttgatcgatgacgacgacgagcttttcattgtcgatcgtttgaaagagcttatcaagtacaaaggttttcaagtggctccggctgagctagaggctctcctcatcggtcatcaggatatcaccgacgtggccgttgtcgcaatgaaggaagaggctgctggtgaagttcccgttgcgtttgttgtgaaatccaaggattcagagttatcggaagatgatgtgaaacaattcgtggcgaaacaggttgtgttttacaagagaatcaacaaagtgttcttcgttgagtccattcctaaagctccgtcagggaagatattgaggaaagacctgagggcaaaactagcaaatgggttggtaaactaa

SEQ ID NO: 30 - protein, 551 aa, Brassica napus
MAPQEDAMQKQSSNKSDVIFRSKLPDIYIPNHLPLHDYIFQNISEFASKPCLINGPTGHVYTYSEVHVASRRIAAGFQKLGVNQNDVVMILLSNCPEFVLSFLAASFRGATATAANPFFTPAEIAKQAKASNSKLIVTESRYVDKIKDLQNDGVIIVCTDEEPSPIPEGCLRFTELTQSTEMETVEISSDDVVALPYSSGTTGLPKGVMLTHKGLVTSVAQQVDGDNPNLYFHSDDVILCVLPLFHIYALNSIMLCGLRVGASILIMPKFEINLLLELIQRCKVTVAPMVPPIVLAMAKSPETEKYDLSSIRVVKSGAAPLGKELEDAVSAKFPNAKLGQGYGMTEAGPVLAMSLGFAKEPFPVKSGACGTVVRNAEMKIIDPDTGDSLSKNKPGEICIRGHQIMKGYLNNPAATAETIDKDGWLHTGDIGLIDDDDELFIVDRLKELIKYKGFQVAPAELEALLIGHQDITDVAVVAMKEEAAGEVPVAFVVKSKDSELSEDDVKQFVAKQVVFYKRINKVFFVESIPKAPSGKILRKDLRAKLANGLVN

SEQ ID NO: 31 - DNA, 996 bp, Glycine max
  CDS (1)..(996): peroxisomal-coenzyme A synthetase (GM50942269)
atgaccatgcttatggttggaggttgccatgtcttgatgccaaaatttgatgcagaatcagctgttgatgccatagagcaatatgcggtgacatcttttatcacagttcctgcaataatggctagtctgatttctataattaggcacaaagagacctggcaagggggagacactgtcaagaaaattcttaatgggggtggaagcctctcacatgagctcatcaaggacactagcatattcttccacaaagctaaacttatttcagcttatgggatgacagagacatgttcttcattgacattcctgacactctatgagccaatgcatgaaacaacaagccagtcccttcaagcatttggtgtggcaggatcaaagctcattcaccagcaacaaggtgtttgtgttggcaaggctgcaccacatatagaactaaagataagtgcagatgcttctggtcacattgggagaattctaactagaggaccacatataatgctaaggtattgggaccaaactctcacaaatccattaaatccgaataatgaagcctggcttgacacaggtgacattggatcaattgatcattatggtaatttgtggctccttggtcgaacaaatggtcgaatcaagagtggtggggagaacatttaccctgaagaggttgaggcaattctacaacaacatccagggattgcaagtgttgttgttgtgggaatcccagatgctcacctgacagagatggtagcagcatgtatccaactaagggaaaattggcaatggtcagagcagttgtctgcttcaaatgaagagtttctcctatctagaaagaatctttatcaatattgtttagaaaatcatttaagcaggtttaagataccaaaaacgtttattgtatggaggaagccatttcaactcaccacgacagggaaaataagaagagaccaagtcagaaaagaagttatgtctcagctgcaatctttgcatagtaatctttga

SEQ ID NO: 32 - protein, 331 aa, Glycine max
MTMLMVGGCHVLMPKFDAESAVDAIEQYAVTSFITVPAIMASLISIIRHKETWQGGDTVKKILNGGGSLSHELIKDTSIFFHKAKLISAYGMTETCSSLTFLTLYEPMHETTSQSLQAFGVAGSKLIHQQQGVCVGKAAPHIELKISADASGHIGRILTRGPHIMLRYWDQTLTNPLNPNNEAWLDTGDIGSIDHYGNLWLLGRTNGRIKSGGENIYPEEVEAILQQHPGIASVVVVGIPDAHLTEMVAACIQLRENWQWSEQLSASNEEFLLSRKNLYQYCLENHLSRFKIPKTFIVWRKPFQLTTTGKIRRDQVRKEVMSQLQSLHSNL

SEQ ID NO: 33 - DNA, 1008 bp, Glycine max
  CDS (1)..(1008): peroxisomal-coenzyme A synthetase (GM59534234)
atgtcaatccttaacatatatgatatacatgttcatgggcttttcaatggtttaatggcccctctctatgcaggttccacggttgagtttttgccaaaatttagtgtgagaggagtttggcagagatggcgtgagtcatatccaaccgatggttctaaggctgaagaggctataactgtgttcactggagttccaactatctatgcccgattgatacaaggttatcatgcaatggatcctgagctacaagctgcttcagtatctgctgcaaagaacttgcgtcttatgatgtgtgggtcctctgcacttccactgcctgttatgcaagaatgggaagccatcactgggcatcgcttattggaacgatatggcatgactgaatttgttatggcattgtcgaatccattgaaaggtgagcgcaaacctggaactgttggcaaaccatttcccggtatacaggtcaagattattacagatgaagagagtgtgaatggaaatactggaatgggtgagctttgcattaaaagcccttcattgtttaaagaatattggaaacttcctgaggcaacaaaggaatcatttactgatgatggattcttcaagactggagatgcagttactacagatgaagatggatatttcatcattctaggacgtactaatgctgatataatcaaggctggtggatataagctttctgcattagaaattgaatcggttataatagagcatccagctgtctcagaatgttgcgttttaggcctaccagacaaagactatggagagattgttagtgcaatcgttgtgccagaggccgatgttaaacgaaaacaagatcaagaatcaaagcctgttctaagcctagaagaattatccaactgggccaaagacaaaattgcaccttacaagattccaactcagctaatcgtgtgggataaactccctcgcaatgccatggggaaggtaaataaaaaagagctgaagaagctgcttgtttcagaacagtga

SEQ ID NO: 34 - protein, 335 aa, Glycine max
MSILNIYDIHVHGLFNGLMAPLYAGSTVEFLPKFSVRGVWQRWRESYPTDGSKAEEAITVFTGVPTIYARLIQGYHAMDPELQAASVSAAKNLRLMMCGSSALPLPVMQEWEAITGHRLLERYGMTEFVMALSNPLKGERKPGTVGKPFPGIQVKIITDEESVNGNTGMGELCIKSPSLFKEYWKLPEATKESFTDDGFFKTGDAVTTDEDGYFIILGRTNADIIKAGGYKLSALEIESVIIEHPAVSECCVLGLPDKDYGEIVSAIVVPEADVKRKQDQESKPVLSLEELSNWAKDKIAPYKIPTQLIVWDKLPRNAMGKVNKKELKKLLVSEQ

SEQ ID NO: 35 - DNA, 999 bp, Glycine max
  CDS (1)..(999): peroxisomal-coenzyme A synthetase (GM59654631)
atgaggtctatgccggtatatttatggtgtgttcccatgtttcactgcaatggttggtgcctcccttgggccattgctgctcagggtggcactaacgtctgccaaagaagtgtcaccgctgaagggatatttcacaatatttttaggcacaaggttactcacatggggggtgcaccgacagttttaaacatgataatcaattcaccacccaaagtccggaagccgcttccgggaaaggtcgaagtgatgacaggtggtgcaccaccgcctccggatgtgattatcaggatggaagaactaggatttaatgtgactcattcatatggtttgacagaaacctatggtccaggatcaatttgcacatggaaaccagaatgggacaatctgtctcgagatgcacaagcaaaactcaaggcacgtcaaggagtggcccatgttgggatggaagacctagatgtgaaagatcctcacacaatgaagagtgtaccggctgatgcaaaaaccatgggtgaggtgatgttcaggggcaacactgtgatgaatggatatctgaaggacttgaaagcaacacaagaggcatttaaaggtggatggttttggactggtgatttgggagtaaagcatcctgatggatacatagagcttaaggatcgctcgaaggacatcatcatctctgggggagaaaatattagcacaattgagttggaaggagtgatttttagtcatccggcagtttttgaggcagctgttgttgggagacctgatgattattggggagagacaccttgtgcatttgtcaaactgaaggaggggtgcagtgctacatcagaggagataatacaattttgtcagaatcgattgcctcgctttatggctcctcgaactgtggtgtttactgatctgccaaagacatcaactggcaagacacagaaatttgttctgagggagaaggcaaaggccatgggaagcctgactaagaagaacactagccggttataa

SEQ ID NO: 36 - protein, 332 aa, Glycine max
MRSMPVYLWCVPMFHCNGWCLPWAIAAQGGTNVCQRSVTAEGIFHNIFRHKVTHMGGAPTVLNMIINSPPKVRKPLPGKVEVMTGGAPPPPDVIIRMEELGFNVTHSYGLTETYGPGSICTWKPEWDNLSRDAQAKLKARQGVAHVGMEDLDVKDPHTMKSVPADAKTMGEVMFRGNTVMNGYLKDLKATQEAFKGGWFWTGDLGVKHPDGYIELKDRSKDIIISGGENISTIELEGVIFSHPAVFEAAVVGRPDDYWGETPCAFVKLKEGCSATSEEIIQFCQNRLPRFMAPRTVVFTDLPKTSTGKTQKFVLREKAKAMGSLTKKNTSRL

SEQ ID NO: 37 - DNA, 717 bp, Glycine max
  CDS (1)..(717): peroxisomal-coenzyme A synthetase (GM59778298)
atgtctgaacgtggatttcgtgtcacacacacttatggtctctcagaaacctatggcccctctgtctactgtgcctggaaaccagaatgggaatcacttccccctgaaaaccaagcccggctcaatgcaagacaaggtgttaggtacattggcttggaaggcctggctgttgtgaacacaaaaaccatggaacctgttcctgctgatggtaaaactgttggtgagattgtgatgcggggtaattctgtgatgaaaggctacttaaagaaccctaaggctaacgaggagacctttgcaaatggatggtttcattctggggatcttgctgtgaagcacccagatggatatatagaaattaaagacagatcaaaggacatcataatctctggtgctgaaaacatcagcagtgtagagatagagaatactctttactcgcaccctgcaatactggaagcagcagtggttgcaagggcagatgaaaaatggggcgagtctccctgcgcttttgtgacattaaagccaggagtggataaaagcaatgagcagcgtataattgaggatatactaaagttcagcagggccaagatgcctgcttattgggttccaaaatcagttgtgtttggagccttacctaagacagctactgggaagatacagaagcatatactgagggccaaggcaaaagagatgggacctgtaaagttgagcaagttataa

SEQ ID NO: 38 - protein, 238 aa, Glycine max
MSERGFRVTHTYGLSETYGPSVYCAWKPEWESLPPENQARLNARQGVRYIGLEGLAVVNTKTMEPVPADGKTVGEIVMRGNSVMKGYLKNPKANEETFANGWFHSGDLAVKHPDGYIEIKDRSKDIIISGAENISSVEIENTLYSHPAILEAAVVARADEKWGESPCAFVTLKPGVDKSNEQRIIEDILKFSRAKMPAYWVPKSVVFGALPKTATGKIQKHILRAKAKEMGPVKLSKL

SEQ ID NO: 39 - DNA, 312 bp, Saccharomyces cerevisiae
  CDS (1)..(312): histone H4 (YNL030W)
atgtccggtagaggtaaaggtggtaaaggtctaggaaaaggtggtgccaagcgtcacagaaagattctaagagataacattcaaggtatcactaagccagctatcagaagattagctagaagaggtggtgtcaagcgtatttctggtttgatctacgaagaagtcagagccgtcttgaaatccttcttggaatccgtcatcagggactctgttacttacactgaacacgccaagagaaagactgttacttctttggatgttgtttatgctttgaagagacaaggtagaaccttatatggtttcggtggttaa

SEQ ID NO: 40 - protein, 103 aa, Saccharomyces cerevisiae
MSGRGKGGKGLGKGGAKRHRKILRDNIQGITKPAIRRLARRGGVKRISGLIYEEVRAVLKSFLESVIRDSVTYTEHAKRKTVTSLDVVYALKRQGRTLYGFGG

SEQ ID NO: 41 - DNA, 321 bp, Linum usitatissimum
  CDS (1)..(321): histone H4 (LU62237699)
atgtcaggaagaggcaaagggggaaagggacttggaaagggaggagccaagaggcacaggaaggtcttgcgagataacatccagggcatcaccaagcccgccatccgaagactcgcccgcagaggtggggtcaagcgtatcagtggcctcatctacgaggaaaccagaggcgttctcaagatcttcctcgagaacgtcattcgcgatgctgtcacctacactgagcacgctcgcaggaagaccgtcaccgcaatggatgtcgtctacgctttgaagaggcaaggcgtaccctctacggttcggtggttgaggcgcccttga

SEQ ID NO: 42 - protein, 106 aa, Linum usitatissimum
MSGRGKGGKGLGKGGAKRHRKVLRDNIQGITKPAIRRLARRGGVKRISGLIYEETRGVLKIFLENVIRDAVTYTEHARRKTVTAMDVVYALKRQGVPSTVRWLRRP

SEQ ID NO: 43 - DNA, 318 bp, Oryza sativa
  CDS (1)..(318): histone H4 (OS36075085)
atgactggacgcggcaagggtggcaagggcctcggcaagggcggtgccaagcgccaccgtaagattcttcgcgacaacatccagggtatcaccaagcccgcgattcgccgcctggcacgccgtggtggtgtcaagcgtatctctgccatgatctacgaggagacacgtgggggccttaagaccttcctcgagggcgtcatgcgtgactttcttttctattccgaacacaccaaacacaagaatggttctttcctctacactctctacaggttgaagaagcaaagacaaaccctttatccgtgtttaagaaaaaaataa

SEQ ID NO: 44 - protein, 105 aa, Oryza sativa
MTGRGKGGKGLGKGGAKRHRKILRDNIQGITKPAIRRLARRGGVKRISAMIYEETRGGLKTFLEGVMRDFLFYSEHTKHKNGSFLYTLYRLKKQRQTLYPCLRKK

SEQ ID NO: 45 - DNA, 594 bp, Saccharomyces cerevisiae
  CDS (1)..(594): SYM1-type integral membrane protein (YLR251W)
atgaagttattgcatttatatgaagcgagcttggagagaaggcccaaaactacgaatgcgataatgacaggtgcgctatttggaattggtgatgtttctgctcaattgttgtttccaacatccaaagtaaacaagggttatgattataaaaggacagctagggctgtcatctatggttctttaattttctcctttataggtgacaagtggtacaagatcttgaacaacaagatttatatgcgtaacagacctcagtaccactggtctaatatggttttacgggtagctgtcgatcaattggcgtttgcgccgctaggtttgccattttatttcacctgtatgtccatcatggaaggtagatcatttgacgtagctaagttgaaaataaaagagcaatggtggcctacacttttgactaattgggcagtttggccacttttccaagcgattaacttttctgttgttcctttacaacataggttactagctgttaatgtcgttgcaatattttggaacacttacttatcttataaaaactcaaaggttatggagaaagacaaggtacctgttcattatccacccgtggtcgaataa

SEQ ID NO: 46 - protein, 197 aa, Saccharomyces cerevisiae
MKLLHLYEASLERRPKTTNAIMTGALFGIGDVSAQLLFPTSKVNKGYDYKRTARAVIYGSLIFSFIGDKWYKILNNKIYMRNRPQYHWSNMVLRVAVDQLAFAPLGLPFYFTCMSIMEGRSFDVAKLKIKEQWWPTLLTNWAVWPLFQAINFSVVPLQHRLLAVNVVAIFWNTYLSYKNSKVMEKDKVPVHYPPVVE

SEQ ID NO: 47 - DNA, 837 bp, Brassica napus
  CDS (1)..(837): SYM1-type integral membrane protein (BN42108421)
atgcttatcgacgccactttcacccggagaacaccactcactctcagttctcttggtgtttccggtaatcacaatcgaaagattttaaccggagatggttcaagcagggctctttcgtttggttacaaaaatggttcactgagttctgctcgtatcaactgttctggtcgttccgggactgggtttggccatctgggtcgggtctcttcggtctcaggtggaggttcaggtgattccggcggaattggtggttcaggtggtggtgggggtggtgatagttccggcggcggaaacggaaacaagtggtcatttctgtcatggtacttggctcttctctcgaattatcctgttttgaccaaatctgtgacatcagcaattttgacactcattggtgatttgatctgtcagcttacaatcaatagaacctcatctctggacaaaaagaggacactcacgtttaccatattgggattaggactagtcggtccagcattgcatttctggtatttgtatttgagcaaagtggtgacagcttccggattatcaggcgcagttctgagacttttactggaccagtttgtttttgctcctgtttttgttggagttttcttatcagctgttgtgacacttgaaggaaaaccatcaaatgtcataccgaagctaaagcaggagtggactggtgcagtggtagcaaattggcagctatggataccgtttcagtttcttaacttcagatttgttccacaaaacttccaggtactggcttccaacgtagtggctttggcttggaatgtgatcttatcattcaaggctcacaaagaagttgtgccaaagtag

SEQ ID NO: 48 - protein, 278 aa, Brassica napus
MLIDATFTRRTPLTLSSLGVSGNHNRKILTGDGSSRALSFGYKNGSLSSARINCSGRSGTGFGHLGRVSSVSGGGSGDSGGIGGSGGGGGGDSSGGGNGNKWSFLSWYLALLSNYPVLTKSVTSAILTLIGDLICQLTINRTSSLDKKRTLTFTILGLGLVGPALHFWYLYLSKVVTASGLSGAVLRLLLDQFVFAPVFVGVFLSAVVTLEGKPSNVIPKLKQEWTGAVVANWQLWIPFQFLNFRFVPQNFQVLASNVVALAWNVILSFKAHKEVVPK

SEQ ID NO: 49 - DNA, 834 bp, Glycine max
  CDS (1)..(834): SYM1-type integral membrane protein (GMsf23a01)
atggtgtatggaggagcaatgtgcgctcacgcgcacaaattcctactctcgccccgtgccctcaacctttgccgtcaaaacaccactcgccttcaacaatttcaaactctcactccgctccactctcaatcccttcctttcaacttcaaaaactccgccacccccttcaaacccaccctcatccgcctctccgccctctccaacgatggctccggcggaaacggcccacatggcggcgccggcggaggcggaggctctgacggtcccaattctggcggcgccggcgatggaggaggcaagtggtccttcttgtcatggtacttggctcttcttggaaaataccctgttgccgtgaaagctctaacatcttcaattttgaatctaattggagatttgatttgtcagcttgtgatagaccaagtgccgtcactggatttcaagaggacatttgtctttacttttcttggttttgctttagtgggtccaacactgcatttctggtatttgtatctgagtaaattggttacacttcctggagcatcaggtgcacttttacggcttgtacttgatcagttcttattttcacccatattcatcggagttttcttatctacattggtgacactggagggaaacccatcacgagctgtacccaagcttaaacaggagtggttttctgcagttctagcaaactggaaactatggataccttttcaatttctcaacttcagatttgttccacaacaatttcaggtccttgctgccaatgttattgctttggtgtggaatgttattctctcatttatggcacataaagaggttcttccaaaatag

SEQ ID NO: 50 - protein, 277 aa, Glycine max
MVYGGAMCAHAHKFLLSPRALNLCRQNTTRLQQFQTLTPLHSQSLPFNFKNSATPFKPTLIRLSALSNDGSGGNGPHGGAGGGGGSDGPNSGGAGDGGGKWSFLSWYLALLGKYPVAVKALTSSILNLIGDLICQLVIDQVPSLDFKRTFVFTFLGFALVGPTLHFWYLYLSKLVTLPGASGALLRLVLDQFLFSPIFIGVFLSTLVTLEGNPSRAVPKLKQEWFSAVLANWKLWIPFQFLNFRFVPQQFQVLAANVIALVWNVILSFMAHKEVLPK

SEQ ID NO: 51 - DNA, 486 bp, Hordeum vulgare
  CDS (1)..(486): SYM1-type integral membrane protein (HV62697288)
atgatcactgctgggttcctcgcaggcatcagtgactccgtggcgcagaagctctctggatatcagaagattgagaagcgccgcctcctgctcaagatgatatttgggtttgcatatggtggcccatttgggcatttcctgcataaaatgttggattatatctttaaagggaagaaggataccaaaactgtagctaagaaggtgttgctggagcagatcacttcctcaccctggaacaatttactcttcttattctattatggatatgttgttgagaagaggcctttcaaggaggtgaaaattagggtgaagaaacagtatctgtcagtgcaattgtctgcttggatgttttggccagtagttggttggataaaccatcagtacgtgcctttgcagtttcgggtgattgtccacagctttgttgcatgttgctgggggatatttctcaaccttcgcgccagagcgatgtctttgaagcagtcatag

SEQ ID NO: 52 - protein, 161 aa, Hordeum vulgare
MITAGFLAGISDSVAQKLSGYQKIEKRRLLLKMIFGFAYGGPFGHFLHKMLDYIFKGKKDTKTVAKKVLLEQITSSPWNNLLFLFYYGYVVEKRPFKEVKIRVKKQYLSVQLSAWMFWPVVGWINHQYVPLQFRVIVHSFVACCWGIFLNLRARAMSLKQS

SEQ ID NO: 53 - DNA, 843 bp, Linum usitatissimum
  CDS (1)..(843): SYM1-type integral membrane protein (LU61649286)
atgatgatgtcgatgatgactacggtgccgcaaagcccagtatccctcacccgcaaaaccctgctcttcggcggcctaactcgctcgccgtctcctcattccttctgctctaactccgttcatataactggttttctgtttacggatttggattcttctgaatatggatttaggcagctgggtcgtcatcatttacgggtatcggctgtttccggtggaggagggtctggtggtggtgccggcgggataggaggctccggcggagatggaaattccggtggtggaagccccgatggtgggaatcatcactggtctttactttcatggtatctgagccttctagccaaatatcctgttcttaccaaggctgtaacttccggacttctgaattttcttggagacatagtctgccagcttttgattgataaagctccatcaccagacttcaagaggacatcccgattttccttcttgggacttgtacttgttggtcctgctttgcatttctggtatttgtatttgagcaaactggtgacaattcctggagcagcaggtgcatgcactcgtcttatactcgatcagttcctttttgctccggtttttctcggggccttcttatctacattacgtgtgcttgaaggaaggccatcccaagttgtaccaaagctccaacaggagctgttttcttctgttgtagcaaattggcaactgtggatcccgtttcaatttctgaacttcagattcatgccacaacagtttcaggtactgggtgcaaatgtgattgctttggtttggaatgtgatattttcattcaaagcccacaaagagattctgctgaaatag

SEQ ID NO: 54 - protein, 280 aa, Linum usitatissimum
MMMSMMTTVPQSPVSLTRKTLLFGGLTRSPSPHSFCSNSVHITGFLFTDLDSSEYGFRQLGRHHLRVSAVSGGGGSGGGAGGIGGSGGDGNSGGGSPDGGNHHWSLLSWYLSLLAKYPVLTKAVTSGLLNFLGDIVCQLLIDKAPSPDFKRTSRFSFLGLVLVGPALHFWYLYLSKLVTIPGAAGACTRLILDQFLFAPVFLGAFLSTLRVLEGRPSQVVPKLQQELFSSVVANWQLWIPFQFLNFRFMPQQFQVLGANVIALVWNVIFSFKAHKEILLK

SEQ ID NO: 55 - DNA, 882 bp, Oryza sativa
  CDS (1)..(882): SYM1-type integral membrane protein (OS40298410)
atgcccccgaccggagcgctccacgccggcggccgccacctcctcccgctccgccgcgccccgggtgcggcccagcagccatggtcccacgtccgctcccacctcatctcctcctccaagcgcccctccccctccccctccccgccgccgccgccgcccccgccgctgccggttgccccgtctacctccgccttcgtgcagaccgcgggcaggaggagcgggggcggggccggggccggggccgccgccgggtcgacgattttcgcgtggtacctcggctcgatcgaggcgcggcccgtgctgaccaagagcgtcaccgcccgggtcgacgatttcgtgtggtacctcggctcgatcgaggcgcggcccgtgctgaccaagagcgtcaccgccgccgccatcttcaccgtcgccgacctctcctcccagatgatcacacttggccctgaagattcacttgatctagttaggactctgcggatggctagttatgggctgctgatctcaggaccttccttgcatatttggttcaactttgtctcaaaattgctccccaaacaggacgtaatgaacaccttcaagaagatgtttcttgggcaagcggtttatggaccaattattaattcagttttcttctcatacaatgcaggattgcaaggtgagaccatcccagagatcatggcaagactgaagagagatttaatcccgaccatcaaaagtggacttatatactggccactttgtgactttatcactttcaagtttatccccgttcatttacagacgctagtgagcaattccttctcgtttctttggaccatctacataacatacatggctagcttaaagaaagcagatgttgatgtgaccacgagttcataa

SEQ ID NO: 56 - protein, 293 aa, Oryza sativa
MPPTGALHAGGRHLLPLRRAPGAAQQPWSHVRSHLISSSKRPSPSPSPPPPPPPPLPVAPSTSAFVQTAGRRSGGGAGAGAAAGSTIFAWYLGSIEARPVLTKSVTARVDDFVWYLGSIEARPVLTKSVTAAAIFTVADLSSQMITLGPEDSLDLVRTLRMASYGLLISGPSLHIWFNFVSKLLPKQDVMNTFKKMFLGQAVYGPIINSVFFSYNAGLQGETIPEIMARLKRDLIPTIKSGLIYWPLCDFITFKFIPVHLQTLVSNSFSFLWTIYITYMASLKKADVDVTTSS

SEQ ID NO: 57 - DNA, 1437 bp, Saccharomyces cerevisiae
  CDS (1)..(1437): Vacuolar proton pump subunit H (YPR036W)
atgggcgcaaccaaaattttaatggacagtactcatttcaatgagatccgtagtataatccgttcgaggtcagtggcatgggacgccttagccagatctgaggaattgagcgaaattgatgcgtctactgcaaaagcgttagaatccattctggtgaagaagaacattggtgacggtttatcatcttcgaacaatgcacattccgggttcaaagtgaatggcaagacgttgataccattaattcacttactttccacctcagacaacgaagactgcaaaaaatctgtgcagaacctaatagctgaattgttatcgtctgacaagtatggagacgataccgtgaagtttttccaagaagaccccaagcaattggaacaattatttgatgtgtcactcaagggagacttccagactgtgctaatctctgggttcaacgtggtctcactcttagtgcaaaatgggttgcacaatgtgaaactagtggaaaagctgttgaaaaacaacaacttgatcaatatcttgcaaaacattgagcagatggacacttgttacgtgtgcatcagactattgcaagaactggccgtgataccagagtatcgtgacgtgatatggttgcatgagaagaagttcatgcccaccttattcaagatcctgcaacgtgccacggactctcaattggccacgcggatagttgcaacaaactccaaccacctgggtattcaattgcagtaccactctttactattgatatggttgctgacctttaacccagtttttgcaaacgagctagtccagaaatacttgagtgatttcttggacctcttgaaattggttaagataaccataaaggagaaagtgtccagattgtgcatatccatcatcctgcaatgttgctccacgcgcgtcaagcagcacaagaaggtgattaagcaacttttgttgctcggcaacgcgttgcccaccgtacagagcttgagcgaaagaaagtattccgacgaagaattgcgtcaagacatcagcaacctcaaggaaatcctagaaaacgagtaccaagaattgacctccttcgatgaatacgtcgccgaattggactccaagttgctgtgctggtctccaccacatgtcgacaacggtttctggtccgataacattgacgagttcaagaaggacaactacaagatctttagacaattgatcgaactcttgcaagcaaaggtccgtaacggcgacgtcaacgcgaaacaagaaaagatcattatccaagtcgccttgaacgacatcactcacgtggtcgagcttctaccagagagcatcgacgttctcgacaagactggcggcaaagccgacatcatggagttgctgaaccattcagattctagggtgaaatacgaggccctcaaggccacgcaggcaatcattggatataccttcaaataa

SEQ ID NO: 58 - protein, 478 aa, Saccharomyces cerevisiae [sequence omitted]

SEQ ID NO: 59 - DNA, 1353 bp, Brassica napus
  CDS (1)..(1353): Vacuolar proton pump subunit H (BN51362135)
atggatcaagcagaactgtctatggagcaggtgttgaaaagggatattccatgggagacttacatgacgacgaagctcatttcagctacaggtctccagctcttgaggcgctttgataaaaaacctgaaagtgcgagggcacagctgctcgatgaagatggtccagcttatgttcatctgtttgttaccatcttgcgtgatatattcaaggaggaaactgtggaatatgttttggctttgatttacgaaatgctctctgcaaacccaacacgagctcggttattccatgatgaaactttggaacatgaggatacttacgagcctttcttgaggttgctgtcgaagggaaactggttcattcaagaaaaaagctgcaagatccttgcctggataataagtgctaggccaaaagctggtgttattgctaatggagaagcttcgggttctaaaaaacctattactacaattgatgatgttctcaatgggttggtggagtggctttgtgctcagttgaggcaaccttctcatccaactcgtggtgctccaattgctatcagctgcctctcgacactgcttaaggaacctgttgtcagatcatcgtttgttaaggcagatggggtgaagttacttgtccctttaatctcaccagcatccactcagcagtctatccagcttctctacgaaacatgtctctgcatctggcttctttcctactatgaacccgcaatagagtacttggcaacatctaggacaatgcaaaggctcacggaagtggttaagagctcgactaaggaaaaggttgtcagggtggtcatattgacattcaggaacttgcttccaaaaggtacatttggtgcccaaatggttgatcttggactcccacatatcatccacagtctgaaaacacaagcatggagtgacgaggacttgctggatgcactgaaccaactagaagaagggctaaaagacaagatcaagaagctgagttccttcgacaaatataagcaagaggttcttcttggccatcttgactggaacccaatgcacaaagaagccaacttctggcgtgagaatgtcactagctttgaggagaatgacttccagatactcagggttctcctcacaatcctggacacgtcaagtgatccaagatcattggcggtggcatgctttgatatctcgcagttcatacagtaccacccagcggggagagtgatcgtgacagacctcaaggcgaaagaaagagtgatgaaactgatgaaccatgagaacgctgaggttaccaagaacgctctcttatgcattcagaggcttctccttggtgctaagtacgccagcttcttgcaagcttga

SEQ ID NO: 60 - protein, 450 aa, Brassica napus
MDQAELSMEQVLKRDIPWETYMTTKLISATGLQLLRRFDKKPESARAQLLDEDGPAYVHLFVTILRDIFKEETVEYVLALIYEMLSANPTRARLFHDETLEHEDTYEPFLRLLSKGNWFIQEKSCKILAWIISARPKAGVIANGEASGSKKPITTIDDVLNGLVEWLCAQLRQPSHPTRGAPIAISCLSTLLKEPVVRSSFVKADGVKLLVPLISPASTQQSIQLLYETCLCIWLLSYYEPAIEYLATSRTMQRLTEVVKSSTKEKVVRVVILTFRNLLPKGTFGAQMVDLGLPHIIHSLKTQAWSDEDLLDALNQLEEGLKDKIKKLSSFDKYKQEVLLGHLDWNPMHKEANFWRENVTSFEENDFQILRVLLTILDTSSDPRSLAVACFDISQFIQYHPAGRVIVTDLKAKERVMKLMNHENAEVTKNALLCIQRLLLGAKYASFLQA

SEQ ID NO: 61 - DNA, 1512 bp, Synechocystis PCC6807
  CDS (1)..(1512): ATP synthase subunit alpha (SLL1326)
atggtaagcattagacccgacgaaattagcagtattatccgccaacagattgagtcctacgaccaaagcgtccaggtttccaatgtgggaacggtgctccaggtgggggacggtacggctcggatctatgggctggaacaggtaatgtcccaggagttactggaatttgaagatggcaccatcggcattgccctaaacctagaggaggacaatgttggggcggtattgatgggggatggttttggtatccaagagggcagtaccgttaaaaccactggtcagattgcccaaattcccattggggatgccatggtgggccgggtggtggattccctcggtcgtcccatcgacggtaaaggccccatcagttccaccgctacccgtttgttggaatccccggcccctggaattattgaacggaagtccgtttgtgaacctatgcaaaccggcatcaccgccattgatgccatgattcccattggtcggggtcagcgggagttgatcattggggaccgtaagaccggtaaaacggcaatcgccattgacaccatcattaaccagaagtccgaagacgtaatttgtgtttacgtggcgatcggtcaaaaagcttccaccgtcgctcaaatcattgacaccctgacggaaaaaggtgccatggcctataccattgtggtggccgccaacgccaacgaccccgccactctgcaatatttggccccctacaccggtgccaccttggcggaacactttatgtatcaaggcaagagcaccttggtaatctatgacgatttgtccaagcaagcccaggcttaccgtcagatgtccctgttgatgcgtcgtccccccggtcgggaagcttaccccggtgatgtgttctacatccactcccgtttgttggagcgggccgccaaattgagtgatgccctcggcggtggtagcatgaccgccctaccggtgattgaaacccaggctggagacgtatctgcctacattcccaccaacgtaatttccattaccgatggtcaaattttcctttccactgaccttttcaacgctggcttccgtcctgctattaacgctggtatttcagtgagccgggttggttccgccgcccaaaccaaagccatgaaaaaagtggctggtaaattgaaactggaactagctcagtttgctgagttggaagccttctcccagtttgcctctgatttggatgccgccacccaagcccaattggcccggggtcaacggttgcgtcaattgttgaagcaaccagaaaattcccccttgtccgtgtgggaacaggtggccattagctatgccggtttaaatggttacatcgacaccattcctgtggacaaagtgactgaatttgcccagggtctgcgggattacctcaaggccaacaaagccaagtacgtagaaattatcaatagctccaaagctttgaccgacgaagcagaaactttgttgaaagaagggatcaaagaattcacccaaggttttgccgcctaa

SEQ ID NO: 62 - protein, 503 aa, Synechocystis PCC6807
MVSIRPDEISSIIRQQIESYDQSVQVSNVGTVLQVGDGTARIYGLEQVMSQELLEFEDGTIGIALNLEEDNVGAVLMGDGFGIQEGSTVKTTGQIAQIPIGDAMVGRVVDSLGRPIDGKGPISSTATRLLESPAPGIIERKSVCEPMQTGITAIDAMIPIGRGQRELIIGDRKTGKTAIAIDTIINQKSEDVICVYVAIGQKASTVAQIIDTLTEKGAMAYTIVVAANANDPATLQYLAPYTGATLAEHFMYQGKSTLVIYDDLSKQAQAYRQMSLLMRRPPGREAYPGDVFYIHSRLLERAAKLSDALGGGSMTALPVIETQAGDVSAYIPTNVISITDGQIFLSTDLFNAGFRPAINAGISVSRVGSAAQTKAMKKVAGKLKLELAQFAELEAFSQFASDLDAATQAQLARGQRLRQLLKQPENSPLSVWEQVAISYAGLNGYIDTIPVDKVTEFAQGLRDYLKANKAKYVEIINSSKALTDEAETLLKEGIKEFTQGFAA

SEQ ID NO: 63 - DNA, 1167 bp, Linum usitatissimum
  CDS (1)..(1167): ATP synthase subunit alpha (LU61815688)
atgaatgttattggagagcctattgatgagaagggcgaaatctcaaccgagcactttttgcctatccatagagaagctccatcctttgttgagcaggccactgagcagcaaatccttgtgaccggtatcaaggttgttgatctccttgctccataccagaggggaggcaagattggtctttttggtggtgctggtgtcggaaagactgttcttattatggaactgatcaacaatgttgccaaagcccatggtggtttctccgtctttgctggtgttggggagcgtacccgtgagggtaatgacttgtacagggaaatgattgagagtggtgtcattaagctaggcgataagcaggctgacagcaaatgtgctcttgtctacggtcaaatgaatgagcccccgggtgctcgtgctcgtgttggtctcactggactaactgttgcagaacatttccgtgatgctgaaggtcaggatgtgctgctctttgttgacaacattttccgcttcacacaggctaactcagaagtgtctgccttgcttggtcgtatcccatctgctgtcggataccaaccaactcttgctaccgatcttggaggactccaagagcgtattaccaccaccaagaaggggtccattacctctgtgcaagctatttatgtgccagctgatgatttgacagatcctgctcctgctaccacctttgcacacttggatgccacaactgtgttgtcgcgacagatctcagagctgggtatttaccctgcagtggatcccctagattctacatcccgtatgctttctcctcacatcctaggagaggaccactatggaaccgctcgtggtgtgcagaaggttcttcagaactacaagaatcttcaggatatcattgctattttgggtatggatgagctgagtgaagacgacaagttgactgtcgcccgtgcccgtaagattcaaaggttcttgagccagccgttccatgtcgctgaggtcttcaccggtgcccccgggaagtacgtagagttgaaggagagcatccaaagtttccagggtgttttggatggaaagtacgacgacttgtcagaacagtcgttctatatggttggaggaatcgacgaggtgattgccaaggcagagaagattgctaaggaatcagcaacctcgtaa

SEQ ID NO: 64 - protein, 388 aa, Linum usitatissimum
MNVIGEPIDEKGEISTEHFLPIHREAPSFVEQATEQQILVTGIKVVDLLAPYQRGGKIGLFGGAGVGKTVLIMELINNVAKAHGGFSVFAGVGERTREGNDLYREMIESGVIKLGDKQADSKCALVYGQMNEPPGARARVGLTGLTVAEHFRDAEGQDVLLFVDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYGTARGVQKVLQNYKNLQDIIAILGMDELSEDDKLTVARARKIQRFLSQPFHVAEVFTGAPGKYVELKESIQSFQGVLDGKYDDLSEQSFYMVGGIDEVIAKAEKIAKESATS

SEQ ID NO: 65 - DNA, 1452 bp, Synechocystis PCC6819
  CDS (1)..(1452): ATP synthase subunit beta (SLR1329)
atggtagccgtaaaagaagcaactaacgttggcaaaattacccaggtcatcgggcctgtaattgacgcccagttccccagtggtaaattgccccgtatttataatgcccttaaagtccaaggcagaaactctgctggtaacgaagtagctgttacctgtgaagtgcagcagcttctcggcgataaccaagtccgagccgtagccatgagttccaccgacggtctcgtccggggcatggacgtggtagacaccggggcccccatcagcgttcccgtcggcaccggcaccctgggtcgtatttttaacgttcttggtgagcctgttgacaacaaaggccccgtgcccgctggtgaaactttccccattcaccgtcccgctcccaaattggtggatttggaaaccaagccccaagtatttgaaaccggcattaaggtaattgacctgcttactccctaccgtcagggtggcaaaatcggtctcttcggtggtgctggtgtgggcaaaaccgtaatcatgatggaattgattaacaacatcgccatccaacatggtggtgtatctgtatttggtggcgtaggggaacggacccgggaagggaatgacctctacaacgaaatgatcgaatccaacgtaatcaacgccgacaaaccggaagagtccaaaattgctctggtgtacggtcagatgaacgaaccccccggggctcggatgcgggtaggcttaaccgctttgaccatggcggaatatttccgggatgtgaacaaacaggacgtattgctcttcatcgacaacattttccgcttcgtccaagctggttcggaagtatcggctctgttgggccggatgccctctgcggtaggttaccagcccactttaggtacggacgttggtgatttgcaagagcgtatcacctccaccaaggaaggttccattacctccattcaggctgtgtatgtaccggcggacgacttgactgaccccgcccccgccaccacctttgcccacttggacggtaccaccgtgctttcccgtggtttggccgctaaaggtatttaccccgccgtggaccccttggattccaccagcaccatgcttcagccctccatcgttgggtcagagcattacgacaccgctcgggaagtgcaatccaccctgcaacgctacaaagaattgcaagatattattgccattcttggcttggatgaattgtctgaggaagaccgtttgaccgtagaccgggctcggaaaattgagcgtttcctttcccaacccttcttcgtcgccgaagtatttaccggtgcccccggcaagtacgtttccctggctgacaccatcaaaggtttcaaagcgattttggctggtgaattggatgatttaccggagcaagctttctacctggtgggagacattgaagaagctaaggccaaaggtgccaagctcaaagagggttaa

SEQ ID NO: 66 - protein, 483 aa, Synechocystis PCC6819
MVAVKEATNVGKITQVIGPVIDAQFPSGKLPRIYNALKVQGRNSAGNEVAVTCEVQQLLGDNQVRAVAMSSTDGLVRGMDVVDTGAPISVPVGTGTLGRIFNVLGEPVDNKGPVPAGETFPIHRPAPKLVDLETKPQVFETGIKVIDLLTPYRQGGKIGLFGGAGVGKTVIMMELINNIAIQHGGVSVFGGVGERTREGNDLYNEMIESNVINADKPEESKIALVYGQMNEPPGARMRVGLTALTMAEYFRDVNKQDVLLFIDNIFRFVQAGSEVSALLGRMPSAVGYQPTLGTDVGDLQERITSTKEGSITSIQAVYVPADDLTDPAPATTFAHLDGTTVLSRGLAAKGIYPAVDPLDSTSTMLQPSIVGSEHYDTAREVQSTLQRYKELQDIIAILGLDELSEEDRLTVDRARKIERFLSQPFFVAEVFTGAPGKYVSLADTIKGFKAILAGELDDLPEQAFYLVGDIEEAKAKGAKLKEG

SEQ ID NO: 67 - DNA, 831 bp, Synechocystis PCC6803
  CDS (1)..(831): ABC transporter (SLR0977)
atgaaaacttcccccccagaactgattattgaagcaggacgcacggagcgtcagtattggcaagacctatggcgttaccgggaattgttttacaccctggcttggcgggacattgcggtacggtacaaacaaacggcgatcggtatagcttgggccttaatccggccatttttgaccatggtggtgtttacggtggtatttggtaagttggctaatttaccttcggagggggtgccctatcccattctggtgtttgcgggaatgttgccctggcagtttttttccacttcccttagttccgccagcgatagtctaattgccaatgccaatctaatttctaaggtgtattttcctcgcttagtggtgcctaccagtgccgtggtgactagctttgttgattttttaatttctgggatgattatgttggggctgatggcttggtataatttcttgcccagttggcatgtgattacattgcctttcttcattttgattgcctttatggcttccatgggagcagggttatggctttgttccctcaatgtcaaataccgagattttcgctacattgtgccattcattgtccaatttggtttgtacatttccccggtgggttttagtagtaatgtggtgccggaaaaatggcgattgctctattccattaacccgatggtgagtgtaattgatggttttcgttgggcgattttggggggagaatcaactatatttctgccgggttttttgttgtctttactgttggtgatcattatttttataacaggaattctctattttcgtaagatggaacgcaccttcgctgatgtgatttaa

SEQ ID NO: 68 - protein, 276 aa, Synechocystis PCC6803
MKTSPPELIIEAGRTERQYWQDLWRYRELFYTLAWRDIAVRYKQTAIGIAWALIRPFLTMVVFTVVFGKLANLPSEGVPYPILVFAGMLPWQFFSTSLSSASDSLIANANLISKVYFPRLVVPTSAVVTSFVDFLISGMIMLGLMAWYNFLPSWHVITLPFFILIAFMASMGAGLWLCSLNVKYRDFRYIVPFIVQFGLYISPVGFSSNVVPEKWRLLYSINPMVSVIDGFRWAILGGESTIFLPGFLLSLLLVIIIFITGILYFRKMERTFADVI

SEQ ID NO: 69 - DNA, 261 bp, Synechocystis sp.
  CDS (1)..(261): psaK (SSR0390)
atgcatagctttttgttggccaccgccgttcccgccaccctgtcctggagccctaaagttgctggggtgatgattgcttgcaacattttggcgatcgcctttggtaaattgaccatcaaacaacaaaatgtgggcacccccatgccttcctctaacttctttggcggctttggtttaggggctgtgctgggcaccgctagctttggccacatcctcggcgctggagtaattctggggctagccaatatgggagtactttaa

SEQ ID NO: 70 - protein, 86 aa, Synechocystis sp.
MHSFLLATAVPATLSWSPKVAGVMIACNILAIAFGKLTIKQQNVGTPMPSSNFFGGFGLGAVLGTASFGHILGAGVILGLANMGVL

SEQ ID NO: 71 - DNA, 369 bp, Synechocystis sp.
  CDS (1)..(369): ferredoxin (SLL1382)
atgtcccgttcccaccgagttctgatccacgatcgccaaaacgaaaaagactacagcgtgattgtctccgacgaccgttacattctccaccaggcggaagaccaaggttttgagttaccgttttcctgtcgtaatggggcctgtacggcctgtgcggtgcgggttatttctgggcaaatccatcaaccggaggccatgggactatccccggacctacaacggcaaggttatgctcttctctgtgttagttatgcccaatcggatttggaagtggaaacccaagatgaggacgaggtttatgagttacagtttggccgctactttggggctggtcgggtgcgattaggtttgcctttagatgaggactag

SEQ ID NO: 72 - protein, 122 aa, Synechocystis sp.
MSRSHRVLIHDRQNEKDYSVIVSDDRYILHQAEDQGFELPFSCRNGACTACAVRVISGQIHQPEAMGLSPDLQRQGYALLCVSYAQSDLEVETQDEDEVYELQFGRYFGAGRVRLGLPLDED

SEQ ID NO: 73 - DNA, 387 bp, Brassica napus
  CDS (1)..(387): ferredoxin (BN42448747)
atggctgtgcctacgcacaaggtcactgtacacgatcgacaaagaggagtagttcatgagtttgaggttccagaggatcagtacatattgcactcagctgaatctcagaacattactcttccctttgcttgcaggcatggttgctgcactagttgtgctgtacgtgtaaaatctggagagctgaggcagcctcaagcattaggaatatcagcagaactcaagtctcaggggtatgcacttctttgtgtgggtttccctacatctgaccttgaagtagaaacgcaagatgaggacgaggtctactggctgcaatttggaagatacttcgctcgtggaccaattgaaagagacgattacgcccttgaactcgcaatgggagacgagtag

SEQ ID NO: 74 - protein, 128 aa, Brassica napus
MAVPTHKVTVHDRQRGVVHEFEVPEDQYILHSAESQNITLPFACRHGCCTSCAVRVKSGELRQPQALGISAELKSQGYALLCVGFPTSDLEVETQDEDEVYWLQFGRYFARGPIERDDYALELAMGDE

SEQ ID NO: 75 - DNA, 540 bp, Glycine max
  CDS (1)..(540): ferredoxin (GM49779037)
atggcacttccccttcttcgaatccccggcatgccactcaccaaccaacaccaatcattcccttccaatacccgccgcaaagccacgacggcgaaggcggagctcgggacggcggtggcgcggacgggcggagccggttaccaatctccgtccgtcgacgttcccacgcataaggtcacagtccacgacagacaacgaggaatcgttcacgagttcgtcgtgcctgaggaccagtatatattacatactgctgaggcccagaatattacccttccattcgcctgcaggcatggttgttgtactagctgtgctgtacgtataaagaagggacaaattaggcaaccagaggcacttgggatatctgccgaattgagagacaagggttatgcacttctttgtgtgggcttcccgacctctgatgttgaagtggaaactcaagatgaagatgaggtatattggcttcaatttggacgttattttgcccgaggaccagtggaaagagatgactatgccttggagttggccatggctgacgagtaa

SEQ ID NO: 76 - protein, 179 aa, Glycine max
MALPLLRIPGMPLTNQHQSFPSNTRRKATTAKAELGTAVARTGGAGYQSPSVDVPTHKVTVHDRQRGIVHEFVVPEDQYILHTAEAQNITLPFACRHGCCTSCAVRIKKGQIRQPEALGISAELRDKGYALLCVGFPTSDVEVETQDEDEVYWLQFGRYFARGPVERDDYALELAMADE

SEQ ID NO: 77 - DNA, 513 bp, Synechocystis sp.
  CDS (1)..(513): flavodoxin (SLL0248)
atgacaaaaattggacttttttacggtactcaaaccggcaacactgaaaccattgctgaactgattcaaaaagaaatgggcggcgatagtgtggtcgatatgatggatatatcccaggctgatgttgatgattttaggcaatatagttgcctgattatcggttgtcccacctggaatgtgggggaactccagagtgattgggaaggcttttatgaccaattagacgaaattgattttaatggcaaaaaagtagcctattttggtgctggcgatcaggttggttatgcagataattttcaagacgccatgggcattttagaagaaaaaatcagtggattaggcggtaaaacagtggggttttggcccaccgctggctatgattttgacgaatcaaaagcggtgaaaaatgggaaatttgttggtttagctttggacgaagataatcagccagagttaacagaattaagagtaaagacatgggtaagtgaaattaaaccaattttgcaatcctaa

SEQ ID NO: 78 - protein, 170 aa, Synechocystis sp.
MTKIGLFYGTQTGNTETIAELIQKEMGGDSVVDMMDISQADVDDFRQYSCLIIGCPTWNVGELQSDWEGFYDQLDEIDFNGKKVAYFGAGDQVGYADNFQDAMGILEEKISGLGGKTVGFWPTAGYDFDESKAVKNGKFVGLALDEDNQPELTELRVKTWVSEIKPILQS

SEQ ID NO: 79 - DNA, 498 bp, Synechocystis sp.
  CDS (1)..(498): photosystem I reaction center subunit III (SLL0819)
atgaaacatttgttggcgttgctcctagcctttacactctggtttaatttcgctccctcagcttcggcggacgattttgccaatttgaccccctgtagcgaaaaccccgcttacttggccaagtctaaaaacttcctcaataccaccaacgatcccaactccggtaaaattcgggcggaacgttatgcctctgccctctgtggccccgaaggttatccccacctgattgtggacggtcgtttcacccacgctggtgactttttgattcctagcattttgttcctgtacattgctggttggatcggctgggttggtcgttcttacctgattgaaattcgggaaagcaaaaatcctgaaatgcaggaagtggttattaatgtccccctagcgatcaaaaaaatgttgggtggtttcctttggcccttggccgccgttggtgaatacacctccggcaaactggtgatgaaggattcagaaatccccacttccccccgctaa

SEQ ID NO: 80 - protein, 165 aa, Synechocystis sp.
MKHLLALLLAFTLWFNFAPSASADDFANLTPCSENPAYLAKSKNFLNTTNDPNSGKIRAERYASALCGPEGYPHLIVDGRFTHAGDFLIPSILFLYIAGWIGWVGRSYLIEIRESKNPEMQEVVINVPLAIKKMLGGFLWPLAAVGEYTSGKLVMKDSEIPTSPR

SEQ ID NO: 81 - DNA, 654 bp, Brassica napus
  CDS (1)..(654): photosystem I reaction center subunit III (BN51362302)
atgtctctcacgatcccgacgaacctcgtcctcaacccgagacccaacaagaagactctcactcaatccttacctaaatccaccgcgagattcgtctgctccaccgatgacaacaagtccatgaaggcattctcagcggcggtggctctctcttccatcctcctctcatctccgatgccagccgccgctgatatctcgggcctaaccccttgcaaggagtcgaaacagttcgccaagagggagaagcaacagatcaagaagctccaatcatctctcaagctctacgctcctgagagtgctcctgctcttgctcttaacgctcagatcgagaagaccaaacgcaggttcgacaactacggaaagtacgggcttctgtgcggtgcagacgggctaccacacctgatagtgaacggagaccagaggcattggggagagttcataacacctggacttttgttcctctacattgcgggatggatcgggtgggtggggagaagctacttgatagctattagtgatgagaagaaaccagcgatgaaagagatcatcattgatgttccattggctagtcgtctcatcttccgtggtttcatttggcctgttgctgcttacagagccttgctcaatggcgatctcattgccaaggatgtctaa

SEQ ID NO: 82 - protein, 217 aa, Brassica napus
MSLTIPTNLVLNPRPNKKTLTQSLPKSTARFVCSTDDNKSMKAFSAAVALSSILLSSPMPAAADISGLTPCKESKQFAKREKQQIKKLQSSLKLYAPESAPALALNAQIEKTKRRFDNYGKYGLLCGADGLPHLIVNGDQRHWGEFITPGLLFLYIAGWIGWVGRSYLIAISDEKKPAMKEIIIDVPLASRLIFRGFIWPVAAYRALLNGDLIAKDV

SEQ ID NO: 83 - DNA, 663 bp, Brassica napus
  CDS (1)..(663): photosystem I reaction center subunit III (BNDLM1779_30)
atgtcgctcacgatcccgacgaacctcgttctcaacccgagatccaacaaatctctcactcaatccttgcctaaatccaccgcgagattcgtttgctccgatgacaaatccgcgacgcagcaacagtccatgaaggctttctccgccgcagtcgctctctcttccatcctcctatcagctccgatgccagccgtcgctgatatctcggggttgacaccttgcaaggagtcgaaacagttcgccaagagggagaagcaacagatcaagaagcttcaatcatctcttaagctctacgctcctgaaagtgctcctgctcttgctcttaacgctcagatcgagaagaccaaacgcaggttcgacaactacggcaagtatggacttctctgtggcgcagacggtctaccccacctgatagtgaacggagaccagcggcattggggagagttcataactccaggccttctcttcctttacattgctggatggatcgggtgggttggacgaagctacttgatagcaatcagcgatgagaagaaaccagcaatgaaagagatcatcattgatgttccattggctagtcgcctcatcttccgtggtttcatttggcctgttgctgcttacagagcgttactcaatggcgatctcattgccaaggatgtctag

SEQ ID NO: 84 - protein, 220 aa, Brassica napus
MSLTIPTNLVLNPRSNKSLTQSLPKSTARFVCSDDKSATQQQSMKAFSAAVALSSILLSAPMPAVADISGLTPCKESKQFAKREKQQIKKLQSSLKLYAPESAPALALNAQIEKTKRRFDNYGKYGLLCGADGLPHLIVNGDQRHWGEFITPGLLFLYIAGWIGWVGRSYLIAISDEKKPAMKEIIIDVPLASRLIFRGFIWPVAAYRALLNGDLIAKDV

SEQ ID NO: 85 - DNA, 675 bp, Glycine max
  CDS (1)..(675): photosystem I reaction center subunit III (GMsk95f02)
atgtctctcaccattccctccaacctctccaagcccgccgccctccgccccaaactaagcccaaagctaaggtccgccgccacaaccaccatcgtctgcagcaccaccaacaacaacaacaacaacaacgtgtcctccgacctgaaggcgttctccgccgcgctggccctctcctccatcctcctctccgcccctctccccgccggcgccgacatctcggggctcaccccatgcaaggagtcgaagcagttcgcgaagcgtgagaagcagtcgataaagaagctggagtcgtcgctgaagctctacgaggcgggcagcgcccccgcactggccattaaggcgagcgtggagaagacgaagaggaggttcgacaactacgcgaagcaggggttgctgtgcggcggcgatgggttgccgcacctgatcgtgagcggagaccagaggcactggggtgagttcatcacacccggaatactattcctgtacatctcgggatggatcggctgggtgggtcggagctacctgattgcgatcagggacgagaagaaacccaccatgaaggagatcatcatcgatgttcctctggcttctcgcttgctcttcaggggtttcagctggcccgttgctgcctacagagaactcatcaacggcgacctcattgccaaggatgtctag

SEQ ID NO: 86 - protein, 224 aa, Glycine max
MSLTIPSNLSKPAALRPKLSPKLRSAATTTIVCSTTNNNNNNNVSSDLKAFSAALALSSILLSAPLPAGADISGLTPCKESKQFAKREKQSIKKLESSLKLYEAGSAPALAIKASVEKTKRRFDNYAKQGLLCGGDGLPHLIVSGDQRHWGEFITPGILFLYISGWIGWVGRSYLIAIRDEKKPTMKEIIIDVPLASRLLFRGFSWPVAAYRELINGDLIAKDV

SEQ ID NO: 87 - DNA, 675 bp, Glycine max
  CDS (1)..(675): photosystem I reaction center subunit III (GMso56a01)
atgtctctcaccattccctccaacctctccaagcccgccgccctccgccccaaactaagcccaaagctaaggtccgccgccacaaccaccatcgtctgcagcaccaccaacaacaacaacagcaacaacgtgtcctccgacctgaaggcgttctccgccgcgctggccctctcctccatcctcctctccgcccctctccccgccggcgccgacatctcggggctcaccccatgcaaggagtcgaagcagttcgcgaagcgtgagaagcagtcgataaagaagctggagtcgtcgctgaagctctacgaggcgggcagcgcccccgcactggccattaaggcgagcgtggagaagacgaagaggaggttcgacaactacgcgaagcaggggttgctgtgcggcggcgatgggttgccgcacctgatcgtgagcggagaccagaggcactggggtgagttcatcacacccggaatactattcctgtacatctcgggatggatcggctgggtgggtcggagctacctgattgcgatcagggacgagaagaaacacaccatgaaggagatcatcatcgatgttcctctggcttctcgcttgctcttcaggggtttcagctggcccgttgctgcctacacagaactcatcaacggcgacctcattgtcaaggatgtctag

SEQ ID NO: 88 - protein, 224 aa, Glycine max
MSLTIPSNLSKPAALRPKLSPKLRSAATTTIVCSTTNNNNSNNVSSDLKAFSAALALSSILLSAPLPAGADISGLTPCKESKQFAKREKQSIKKLESSLKLYEAGSAPALAIKASVEKTKRRFDNYAKQGLLCGGDGLPHLIVSGDQRHWGEFITPGILFLYISGWIGWVGRSYLIAIRDEKKHTMKEIIIDVPLASRLLFRGFSWPVAAYTELINGDLIVKDV

SEQ ID NO: 89 - DNA, 363 bp, Synechocystis sp.
atgtttaaattattcaaccaagctagccgaattttttttggcattgccctcccctgtctaattttcctgggggggattttttccctgggtaacaccgccctagccgctgacctagcccatggtaaagctatctttgccggtaactgtgccgcttgtcataatgggggccttaatgccatcaaccccagcaaaaccttaaaaatggcagacttggaagccaatggtaaaaattccgtggcggcaattgtggcccagatcaccaatggtaatggcgctatgcctggctttaagggtcgcatcagtgacagcgacatggaagatgtggcggcctacgtgcttgatcaagcggaaaagggctggtag

SEQ ID NO: 90 - protein, 120 aa, Synechocystis sp.
MFKLFNQASRIFFGIALPCLIFLGGIFSLGNTALAADLAHGKAIFAGNCAACHNGGLNAINPSKTLKMADLEANGKNSVAAIVAQITNGNGAMPGFKGRISDSDMEDVAAYVLDQAEKGW

SEQ ID NO: 91 - DNA, 369 bp, Synechocystis sp.
atgatgaccctcactcccaccatcgaattttttgctgatctccccgaagaactcagcaatgtcagcctccgccgcaattctactaccggcgctcgcactgtggtgatgacctttgaacgtctccaggcgatcgaaaagtttcagagttttacccaaaggtttaacggccatttacgtcttgctgacgaagagggggcgatggaaattgaaccatcgtcagtaaaatttatttttggtggagacgaaggggatgaactgcggggtgcccagtgcagttttgacctagttaaaaatgatcattgggaacgctttatccgtttcatggagcgctatgcggaggccaatggcatgggctaccaagatcgttag

SEQ ID NO: 92 - protein, 122 aa, Synechocystis sp.
MMTLTPTIEFFADLPEELSNVSLRRNSTTGARTVVMTFERLQAIEKFQSFTQRFNGHLRLADEEGAMEIEPSSVKFIFGGDEGDELRGAQCSFDLVKNDHWERFIRFMERYAEANGMGYQDR

SEQ ID NO: 93 - DNA, 792 bp, Synechocystis sp.
atggttgttaggttgcaaaacggattgatgggcaaagtgtatttagttggagcggggcctggggatcctggcttgataactgttaagggtaaaactttgttggaaaatgccgaagcagtggtctatgacgctttggtgagtacagctattttagctatggttaatccccaagccgaattgattgatgcaggcaaaaggaggggcagacacaccaaattacagtcagaaactacacagttattggcgcaattagctgaaaaacacgccgtggtggtgcgcttgaaagggggagatccatttatttttggccgggggggggaagagatggaagacctagtcaaagctggcattgaggtggaagtggtaccgggcattacagcgggcattgctgcccctgcctacgctcaaattcccctcacccatcgggcttacagttcctccgtcacctttgtcacgggccatgaatcggcgggaaaatatcggcctgaagtcaattgggcggcgatcgccaagggctcggaaaccattgtgatttatatgggggtgtacagtttggccaccattttgccccagttaatgctggcggggttgggggaagatactcccattgccctaattcgttggggaacctgtccagaacaacagaaactagttggcacttttgccaccattttggcccagattgaagtagaaaattttcaagccccggcgatcgtggtcatcggggcagtggttaattatccggctaatctccgtcagcaattggcacctatactgggcggtgtcaattaa

SEQ ID NO: 94 - protein, 263 aa, Synechocystis sp.
MVVRLQNGLMGKVYLVGAGPGDPGLITVKGKTLLENAEAVVYDALVSTAILAMVNPQAELIDAGKRRGRHTKLQSETTQLLAQLAEKHAVVVRLKGGDPFIFGRGGEEMEDLVKAGIEVEVVPGITAGIAAPAYAQIPLTHRAYSSSVTFVTGHESAGKYRPEVNWAAIAKGSETIVIYMGVYSLATILPQLMLAGLGEDTPIALIRWGTCPEQQKLVGTFATILAQIEVENFQAPAIVVIGAVVNYPANLRQQLAPILGGVN

SEQ ID NO: 95 - DNA, 594 bp, Synechocystis sp.
atgctctggccctataagaccccaggaattacagatggcctgtttgaacgattaccgggcattcccctcagcaaaagggaggtacgcctgctgattatttccgctttgcaattgaaggaggaatccatcatttgggatattggggctggcactggcaccattccggtggaactagccctactgtgtccccgcagtcgcattgtggcggtggaaagggatgaggaggtggcgggtttaattcgccgtaactgcgaacggtttggggtaactaatgtgactgtccatgagggcagtgctccggattgcttaccgcagttaattgcccaccccacccatatttgcattgagggagggcgtcccattaagcaaattttgcaggaaacctgggcccatttagctcccggaggtcgtttagtggcaacagcgaataatttggaaagtctctatggcatttctgaaaccctgtcggaactccaggtccgcaacgtggaggtggtgcaagcgggggtaaatcgtctagaaaaacgtggtttacaacagatttttgcagcggtggaccctacttttattttggcgggagaaaagttgtcttag

SEQ ID NO: 96 - protein, 197 aa, Synechocystis sp.
MLWPYKTPGITDGLFERLPGIPLSKREVRLLIISALQLKEESIIWDIGAGTGTIPVELALLCPRSRIVAVERDEEVAGLIRRNCERFGVTNVTVHEGSAPDCLPQLIAHPTHICIEGGRPIKQILQETWAHLAPGGRLVATANNLESLYGISETLSELQVRNVEVVQAGVNRLEKRGLQQIFAAVDPTFILAGEKLS

SEQ ID NO: 97 - DNA, 1278 bp, Synechocystis sp.
atgatccatgtggtgggcattggcctgaatggtgccgaagggttaactagttcaacactagaattaattgcccaagcaaaaatattggccggcggcgatcgccatttgagttacttcccgcagtacggcaaaaaaagtttagtcatcaaggatttttcagcagatttaaagaaaatcaagcagtttcatcagactttaaagtcacacgaaactattgttgttttagcttccggtgatcccctgtactttggcttgggacggttattactggaaaaattttccccggaacaattaaaatttcatccccatttgagttccattcagctagcctttaatcgtttaaaaatgccctggcaggatgccaccatcatcagtgcccacggaagatccaacgagttgctcatacaagccctgcaaaaaggagcaaaaaaattagccattctcaccgatgggcaaaatcatccaggggcgatcgccaatttatgtttatctttggggttgaccaccacttaccaagcttgggtttgtgaaaacttagcggcaacaaatgaacagattcaagcctttgatttgccatctttagcatccctaaccgcaacggatttttcccccctcaatgtagtggtgctagttaaacaaaagccagaaaatcccctaattgatctggatcagttacccattttgggcatcgccgatcatcactttgctagctttgacgatcgccctggcatgattaccaaacagcctatccgggtgcaaattttagccgctttatccttgcaaccccgacaaattatttgggacattggggccggtaccgggtcagtggcgatcgagggtgcccggttgtgtccccagggaaaagtttttgccattgaaaaaactagcgctggtcaacggttaatcgaacaaaattgccaacgatttcaactgcaaaacgtcgaggtagtggccggctctgccccggaagttttagccaatttacctactcctaaccgagttttcattggtggtaacggtgggcaattaacctctattttgcagacttgcggggaacgtttagcgataaatggtttggtggtgatggcgatcgccagtttggagcatttgagcttagccctgggctggtttaaacaacaacaatggcaggtgaaagtgcaacaggtgcaaattagccagagcgttaaatttgccgaactgacccgctttgatcccctcaatccgatttatttactcactgcggggaggaactccatgggaaacgacgaccttggttaa

SEQ ID NO: 98 - protein, 425 aa, Synechocystis sp.
MIHVVGIGLNGAEGLTSSTLELIAQAKILAGGDRHLSYFPQYGKKSLVIKDFSADLKKIKQFHQTLKSHETIVVLASGDPLYFGLGRLLLEKFSPEQLKFHPHLSSIQLAFNRLKMPWQDATIISAHGRSNELLIQALQKGAKKLAILTDGQNHPGAIANLCLSLGLTTTYQAWVCENLAATNEQIQAFDLPSLASLTATDFSPLNVVVLVKQKPENPLIDLDQLPILGIADHHFASFDDRPGMITKQPIRVQILAALSLQPRQIIWDIGAGTGSVAIEGARLCPQGKVFAIEKTSAGQRLIEQNCQRFQLQNVEVVAGSAPEVLANLPTPNRVFIGGNGGQLTSILQTCGERLAINGLVVMAIASLEHLSLALGWFKQQQWQVKVQQVQISQSVKFAELTRFDPLNPIYLLTAGRNSMGNDDLG

SEQ ID NO: 99 - protein, 8 aa, Artificial Sequence
  Walker A motif; P-loop nucleotide binding motif found across phyla
  BINDING (1)..(8): ATP/GTP-binding site; Prosite PDOC00017
  MISC_FEATURE (8)..(8): Xaa can either be a serine or a threonine residue
GXXXXGKX

SEQ ID NO: 100 - protein, 4 aa, Artificial Sequence
  GTP-specificity motif found across phyla
  MISC_FEATURE (1)..(1): Xaa can be either an asparagine or threonine residue
  misc_feature (3)..(3): Xaa can be any naturally occurring amino acid
XKXD

SEQ ID NO: 101 - protein, 5 aa, Artificial
  characteristic histone H4 signature sequence
GAKRH

SEQ ID NO: 102 - DNA, 986 bp, Petroselinum crispum
  promoter (1)..(986): Ubiquitin promoter
ctagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

SEQ ID NO: 103 - DNA, 1112 bp, Artificial
  synthetic sequence
  promoter (1)..(1112): Super promoter
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 104 - DNA, 695 bp, Vicia faba
  promoter (1)..(695): USP promoter variant used for expression of yeast genes in plants
ctagactgcagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcataccattaattaag

SEQ ID NO: 105 - DNA, 674 bp, Vicia faba
  promoter (1)..(674): USP promoter used for expression of bacterial genes in plants
caaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagc

SEQ ID NO: 106 - DNA, 102 bp, Arabidopsis thaliana
  CDS (1)..(102)
  transit_peptide (1)..(81): for targeting translational fusions to the mitochondria
  misc_feature (82)..(102): linker sequence used in translational fusions with bacterial genes
atgcagaggtttttctccgccagatcgattctcggttacgccgtcaagacgcggaggaggtctttctcttctcgttcttcgtctctcctttgctcttccatg

SEQ ID NO: 107 - protein, 34 aa, Arabidopsis thaliana
MQRFFSARSILGYAVKTRRRSFSSRSSSLLCSSM

SEQ ID NO: 108 - DNA, 102 bp, Arabidopsis thaliana
  CDS (1)..(102)
  transit_peptide (1)..(81): for targeting translational fusions to the mitochondria
  misc_feature (82)..(102): linker sequence used in translational fusions with yeast genes
atgcagaggtttttctccgccagatcgattctcggttacgccgtcaagacgcggaggaggtctttctcttctcgttcttcggaattccagctgaccaccatg

SEQ ID NO: 109 - protein, 34 aa, Arabidopsis thaliana
MQRFFSARSILGYAVKTRRRSFSSRSSEFQLTTM

SEQ ID NO: 110 - DNA, 419 bp, Spinacia oleracea
  5′UTR (1)..(165): 5′ untranslated region
  CDS (166)..(273): first exon of mature chloroplast transit peptide
  Intron (274)..(350): intron
  CDS (351)..(419): second exon of mature chloroplast transit peptide
gcataaacttatcttcatagttgccactccaatttgctccttgaatctcctccacccaatacataatccactcctccatcacccacttcactactaaatcaaacttaactctgtttttctctctcctcctttcatttcttattcttccaatcatcgtactccgccatgaccaccgctgtcaccgccgctgtttctttcccctctaccaaaaccacctctctctccgcccgaagctcctccgtcatttcccctgacaaaatcagctacaaaaaggtgattcccaatttcactgtgttttttattaataatttgttattttgatgatgagatgattaatttgggtgctgcaggttcctttgtactacaggaatgtatctgcaactgggaaaatgggacccatcagggcccagatcgcctct

SEQ ID NO: 111 - protein, 59 aa, Spinacia oleracea
MTTAVTAAVSFPSTKTTSLSARSSSVISPDKISYKKVPLYYRNVSATGKMGPIRAQIAS

(19) United States
(12) Patent Application Publication
(10) Pub. No.: US 2011/0302673 A1
(43) Pub. Date: Dec. 8, 2011

(54) Transgenic Plants with Increased Yield

(75) Inventors: Bryan D. McKersie, Raleigh, NC (US); Wesley Bruce, Raleigh, NC (US)

(73) Assignee: BASF Plant Science GmbH, Ludwigshafen (DE)

(21) Appl. No.: 13/063,941

(22) PCT Filed: Sep. 15, 2009
(86) PCT No.: PCT/EP2009/061931

§ 371 Date: Mar. 15, 2011

Related U.S. Application Data
(60) Provisional application No. 61/099,224, filed on Sep. 23, 2008.
(60) Provisional application No. 61/107,739, filed on Oct. 23, 2008.
(60) Provisional application No. 61/115,947, filed on Nov. 19, 2008.

(51) Int. Cl. A01H 1/06; A01H 5/10; C07H 21/00; A01H 5/00

(52) U.S. Cl. 800/287; 800/298; 800/320.1; 800/312; 800/314; 800/320.2; 800/320.3; 800/306; 800/290; 536/23.6

(57) ABSTRACT

Polynucleotides are disclosed which are capable of enhancing yield of a plant transformed to contain such polynucleotides. Also provided are methods of using such polynucleotides and transgenic plants and agricultural products, including seeds, containing such polynucleotides as transgenes.

This application claims priority benefit of U.S. provisional patent application Ser. No. 61/115,947, filed Nov. 19, 2008; U.S. provisional patent application Ser. No. 61/107,739, filed Oct. 23, 2008; and U.S. provisional patent application Ser. No. 61/099,224, filed Sep. 23, 2008, the entire contents of each of which are incorporated herein by reference.

### FIELD OF THE INVENTION

This invention relates generally to transgenic plants which overexpress isolated polynucleotides that encode polypeptides, in specific plant tissues and organelles, thereby improving yield of said plants.

### BACKGROUND OF THE INVENTION

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass. For example, water use efficiency (WUE) is a parameter often correlated with drought tolerance. Studies of a plant's response to desiccation, osmotic shock, and temperature extremes are also employed to determine the plant's tolerance or resistance to abiotic stresses.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Plant cell membrane transporters are often affected when water availability is limited. In extreme instances, removal of water from the membrane disrupts the normal bilayer structure and results in the membrane becoming exceptionally porous when desiccated. Under more moderate conditions, stress to the lipid bilayer may result in displacement and configuration changes of membrane transporters, leading to lower efficiency in molecule transport. Water deficit can also increase cellular solute concentration which in turn affects configurations of proteins, including transporter proteins.

Crop yield depends on the health, growth and development of crop plants under varying environmental conditions. Correct targeting and timely delivery of mineral nutrients and organic compounds are essential for plant growth and development. Stress conditions such as drought can severely disrupt the normal transport system in a plant. Genes that stabilize molecule transport under such stress conditions help to maintain homeostasis in the plant.

Regulated molecular transport requires energy for many processes in plants. Ion and proton gradients across cell membranes are one form of stored energy in a plant cell. These gradients are used to drive the transport of other molecules across membranes. An example is the mitochondrial electron transport chain that uses the reduction energy of NADH to move protons across the inner mitochondrial membrane creating a gradient of pH and charge. Another example is the electron transport chain in the chloroplast that enables photosynthesis to use the energy of photons to create a proton gradient across the thylakoid membrane and also to create reduction power in the form of NADPH. In both instances, the energy from the proton gradient across the mitochondrial or thylakoid membrane, called the proton motive force, is converted to chemical energy in the form of ATP by membrane bound ATPases. Primary active transport uses the energy from ATP directly in the transport process through the action of an ATPase that cleaves the terminal phosphate of ATP forming ADP.

ATPases are a class of enzymes that catalyze the decomposition of ATP into ADP and a free phosphate ion or the reverse reaction to generate ATP. The dephosphorylation reaction releases energy, which is used to move solutes across the membrane. Transmembrane ATPases import many of the metabolites necessary for cell metabolism and export toxins, wastes, and solutes that can hinder cellular processes. Besides exchangers, other categories of transmembrane ATPase include co-transporters and pumps.

ATPases can differ in function, structure and in the type of ions they transport. F-ATPases in mitochondria, chloroplasts and bacterial plasma membranes are the prime producers of ATP, using the proton gradient generated by oxidative phosphorylation in mitochondria or photosynthesis in chloroplasts. A-ATPases are found in Archaea and function like F-ATPases. V-ATPases are primarily found in eukaryotic vacuoles, catalysing ATP hydrolysis to transport solutes and lower pH in organelles. V-ATPases function exclusively as proton pumps. The proton motive force generated by V-ATPases in organelles and membranes of eukaryotic cells is then used as a driving force for numerous secondary transport processes. P-ATPases are found in bacteria, fungi and in eukaryotic plasma membranes and organelles, and function to transport a variety of different ions across membranes. E-ATPases are cell-surface enzymes that hydrolyse a range of NTPs, including extracellular ATP.

In contrast to primary active transport, secondary active transport uses the energy from a concentration gradient previously established by the above processes. There are two types of secondary active transport processes, exchange transport (antiport) and cotransport (symport). Amino acid, and sugar transport occur via secondary active transport mechanisms.

ABC (ATP-binding cassette) transporters are membrane spanning proteins that utilize the energy of ATP hydrolysis to transport a wide variety of substrates across extra- and intracellular membranes, including metabolic products, lipids and sterols, and drugs. Within bacteria, ABC transporters mainly pump essential compounds such as sugars, vitamins, and metal ions into the cell. Within eukaryotes, ABC transporters mainly transport molecules to the outside of the plasma membrane or into membrane-bound organelles such as the endoplasmic reticulum and mitochondria.

Electron transport reactions are fundamental to the major energy metabolism processes in plant mitochondria (respiration) and chloroplasts (photosynthesis). In both organelles, the transfer of electrons from one molecule on one side of a cell membrane to another molecule on the opposite side of the membrane creates a proton motive force across the membrane. Although efficient, the electron transfer processes in the plant mitochondria and chloroplasts leak a small percentage of electrons to partially reduce oxygen, forming reactive oxygen species such as superoxide. The formation of superoxide not only wastes cellular energy but can cause oxidative stress that promotes a decline in cell function as a result of damage to membrane lipids, proteins and DNA. In addition, there is potential for energy transfer from an activated chlorophyll molecule in the light harvesting complex to molecular triplet oxygen to form singlet oxygen, which is another precursor of reactive oxygen molecules. The tendency of the photosystems and the light harvesting complex to activate oxygen is increased during periods of stress as a consequence of blockage in the normal metabolic pathway that increase or decrease substrate levels beyond critical thresholds.

Respiration in plant mitochondria transfers biochemical energy from nutrients into adenosine triphosphate (ATP) through a series of catabolic oxidation reduction reactions. Typically sugars, but also amino acids and fatty acids, are used as substrates for the transfer of electrons to oxygen using the released energy to synthesize ATP. The overall reaction for sugars can be simplified as C₆H₁₂O₆+6O₂→>6CO₂+6H₂O with a Δ Hc −2880 kJ. In plant mitochondria, the Kreb's cycle reactions release electrons that are used to reduce NAD to NADH. The redox energy from NADH is transferred by an electron transport chain to oxygen. This transfer of electrons along the protein complexes of the inner membrane releases energy that creates a proton gradient across the membrane. The resultant proton motive force across the mitochondrial membrane is used to synthesize ATP. The energy stored in ATP is used in various cellular processes requiring energy, including biosynthesis and transport of molecules across cell membranes.

Photosynthesis is a complex process by which plants and certain types of bacteria produce glucose and oxygen from carbon dioxide (CO₂) and water using the energy from sunlight. The overall chemical reaction can be expressed simply as 6CO₂+6H₂O (+light energy) C₆H₁₂O₆+6O₂. The numerous reactions that occur during photosynthetic are commonly divided into two stages—the “light reactions” of electron and proton transfer within and across the photosynthetic membrane and the “dark reactions” involving the biosynthesis of carbohydrates from CO₂. Higher plants capture light energy using two multi-subunit photosystems (I and II) located in the thylakoid membranes of chloroplasts. This electron transfer creates a proton gradient across the thylakoid membrane generated that is used for the synthesis of ATP. The light reactions in photosynthesis generate both ATP and NADPH that are subsequently used in biochemical reactions producing sugars, amino acids and other cellular components.

Photosystem I (PS-I) is a multi-subunit complex that uses light energy to drive the transport of the electron donated from Photosystem II (PSII) across the thylakoid membrane to reduce NADP to NADPH. PS-I catalyzes the light-driven electron transfer from plastocyanin, which is located on the lumenal side of the thylakoids, to ferredoxin, which is on the stromal side of the membrane. The PS-I complex has at its center the PsaA/PsaB heterodimer, which contains the primary electron donor—a chlorophyll dimer called P700—and the electron acceptors A0, A1 and FX/A/B. A number of smaller protein subunits make up the rest of the complex. Some of these subunits serve as binding sites for the soluble electron carriers plastocyanin and ferredoxin, while the functions of some of the other proteins are not well understood. A large antenna system of about 90 chlorophylls and 22 carotenoids captures light and transfers the excitiation energy to the center. P700 is re-reduced with the electrons delivered from PS-II by plastocyanin. PsaF, is a plastocyanin docking protein in PS-I that facilitates the binding of plastocyanin or cytochrome c, the mobile electron carriers responsible for the reduction of the oxidized donor P700. U.S. Pat. Application Publication 2008/0148432 discloses use of a PS-I PsaF gene to enhance agronomic traits in transgenic plants.

PS-II, also a multi-subunit protein-pigment complex containing polypeptides both intrinsic and extrinsic to the photosynthetic membrane, uses light energy to oxidize water. PS-II has a P680 reaction center containing chlorophyll a. Within the core of the complex, the chlorophyll and beta-carotene pigments are mainly bound to the proteins CP43 (PsbC) and CP47 (PsbB), which pass the excitation energy on to the reaction center proteins D1 (Qb, PsbA) and D2 (Qa, PsbD) that bind all the redox-active cofactors involved in the energy conversion process. The PS-II oxygen-evolving complex (OEC) oxidizes water to provide protons for use by PS-I, and consists of OEE1 (PsbO), OEE2 (PsbP) and OEE3 (PsbQ). The remaining subunits in PS-II are of low molecular weight (less than 10 kDa), and are involved in PS-II assembly, stabilization, demonization, and photo-protection. PsbW is part of this low molecular weight transmembrane protein complex, where it is a subunit of the oxygen-evolving complex. PsbW appears to have several roles, including guiding PS-II biogenesis and assembly, stabilising dimeric PS-II and facilitating PS-II repair after photo-inhibition. U.S. Pat. Application Publication 2007/0067865 discloses a transformed plant having a nucleic acid molecule comprising a structural nucleic acid which may be a PsbW gene.

Electrons from photosystems are occasionally transferred to molecular oxygen forming superoxide, a precursor of more reactive oxygen intermediates. One of the key points of such transfer is at ferredoxin. Ferredoxins are ubiquitous [2Fe-2S] proteins involved in many electron transfer pathways in plants, animals and microorganisms. Ferredoxin (PetF) is an electron carrier protein in the PS-I electron transport chain. In this chain, ferredoxin transports the electron from the PS-I to ferredoxin-NADP oxidoreductase, which catalyzes the electron transfer from Fd to NADP+ to produce NADPH. In addition reducing equivalents from ferredoxin are used for nitrogen and sulfur assimilation, as well as amino acid and fatty acid metabolism. Ferredoxin also provides reducing equivalents for the activation of chloroplast enzymes by the thioredoxin. High levels of ferredoxin are thought to be critical for plant survival in suboptimal environments. In higher plants, ferredoxin is encoded by a small gene family that has tissue-specific and environmentally regulated expression. The genes encoding the ferredoxin protein are down-regulated by iron deficit, oxidative stress and several environmental stresses, including drought, chilling, salinity and ultraviolet light. The amount of ferredoxin mRNA is redox-regulated at the post-transcriptional level, and consequently strategies to improve stress tolerance in crops using transgenic approaches to increase expression of plant ferrodoxin genes have not been successful. Mitochondria also contain ferredoxin proteins that participate in electron transfer reactions.

Flavodoxin has similar redox potential and functions similarly to ferrodoxin in cyanobacteria and algae, but the gene is not found in any plant genome. Flavodoxin has been implicated in the development of stress tolerance in cyanobacteria and algae. U.S. Pat. No. 6,781,034 discloses that expression of a flavodoxin gene from Anabaena in tobacco produced transgenic plants with increased tolerance of drought, high light intensities, heat, chilling, UV radiation, and the herbicide paraquat.

Chlorophyll is a major component of the light harvesting complex surrounding photosystems I and II. It is structurally similar to and produced through the same metabolic pathway as other porphyrin pigments such as heme. At the center of the ring is a magnesium ion and attached are different side chains, usually including a long phytol chain. Cobalamins are complex small molecules produced exclusively by microorganisms, in a pathway that shares early stages with the biosynthetic pathway of chlorophyll. Both cobalamin and chlorophyll pathways stem from a common precursor, uroporphyinogen Ill. The complexity and the specificity of cobalamin (vitamin B12) itself and its production requires about 30 enzymes that discriminate between specific, but closely related substrates in a chemically intricate pathway. One such enzyme, uroporphyrin-III C-methyltransferase, catalyzes the two successive C-2 and C-7 methylation reactions involved in the conversion of uroporphyrinogen-III to precorrin-2 via the intermediate formation of precorrin-1. This reaction directs uroporphyrinogen-III into cobalamin (vitamin B12) or siroheme biosynthesis. U.S. Pat. Application Publication 2005/0108791 discloses use of a Synechocystis sp. uropoyphyrin III C-methyltransferase (CobA) with a chloroplast targeting peptide to produce transgenic plants with improved phenotype.

Some genes that are involved in stress responses, water use, and/or biomass in plants have been characterized, but to date, success at developing transgenic crop plants with improved yield has been limited, and no such plants have been commercialized. There is a need, therefore, to identify additional genes that have the capacity to increase yield of crop plants.

### SUMMARY OF THE INVENTION

The present inventors have discovered that transformation of plants with certain polynucleotides results in improvement in plant yield when the genes are expressed at appropriate levels and the resulting proteins targeted to the appropriate subcellular location. When targeted as described herein, the polynucleotides and polypeptides set forth in Table 1 are capable of improving yield of transgenic plants.

TABLE 1

| | | Polynucleotide | Amino acid |
|---|---|---|---|
| Gene Name | Organism | SEQ ID NO | SEQ ID NO |
| B0821 | Escherichia coli | 1 | 2 |
| B2668 | E. coli | 3 | 4 |
| B3362 | E. coli | 5 | 6 |
| B3555 | E. coli | 7 | 8 |
| SLL1911 | Synechocystis | 9 | 10 |
| | sp. pcc6811 | | |
| SLR1062 | Synechocystis | 11 | 12 |
| | sp. pcc6818 | | |
| YDL193W | Saccharomyces | 13 | 14 |
| | cerevisiae | | |
| B1187 | E. coli | 15 | 16 |
| B2173 | E. coli | 17 | 18 |
| GM50181105 | Glycine max | 19 | 20 |
| B2670 | E. coli | 21 | 22 |
| YBR222C | S. cerevisiae | 23 | 24 |
| BN51408632 | B. napus | 25 | 26 |
| BN51423788 | B. napus | 27 | 28 |
| BN51486050 | B. napus | 29 | 30 |
| GM50942269 | G. max | 31 | 32 |
| GM59534234 | G. max | 33 | 34 |
| GM59654631 | G. max | 35 | 36 |
| GM59778298 | G. max | 37 | 38 |
| YNL030W | S. cerevisiae | 39 | 40 |
| LU62237699 | Linum usitatissimum | 41 | 42 |
| OS36075085 | O. sativa | 43 | 44 |
| YLR251W | S. cerevisiae | 45 | 46 |
| BN42108421 | B. napus | 47 | 48 |
| GMsf23a01 | G. max | 49 | 50 |
| HV62697288 | Hordeum vulgare | 51 | 52 |
| LU61649286 | L. usitatissimum | 53 | 54 |
| OS40298410 | O. sativa | 55 | 56 |
| YPR036W | S. cerevisiae | 57 | 58 |
| BN51362135 | B. napus | 59 | 60 |
| SLL1326 | Synechocystis sp. | 61 | 62 |
| LU61815688 | L. usitatissimum | 63 | 64 |
| SLR1329 | Synechocystis sp. | 65 | 66 |
| SLR0977 | Synechocystis sp. | 67 | 68 |
| ssr0390 | Synechocystis sp. | 69 | 70 |
| sll1382 | Synechocystis sp. | 71 | 72 |
| BN42448747 | B. napus | 73 | 74 |
| GM49779037 | G. max | 75 | 76 |
| sll0248 | Synechocystis sp. | 77 | 78 |
| sll0819 | Synechocystis sp. | 79 | 80 |
| BN51362302 | B. napus | 81 | 82 |
| BNDLM1779_30 | B. napus | 83 | 84 |
| GMsk95f02 | G. max | 85 | 86 |
| GMso56a01 | G. max | 87 | 88 |
| sll1796 | Synechocystis sp. | 89 | 90 |
| slr1739 | Synechocystis sp. | 91 | 92 |
| sll0378 | Synechocystis sp. | 93 | 94 |
| slr1368 | Synechocystis sp. | 95 | 96 |
| sll0099 | Synechocystis sp. | 97 | 98 |

In one embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide having a sequence selected from the group consisting of SEQ ID NO:2; SEQ ID NO:4; SEQ ID NO:6; and SEQ ID NO:8; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; and an isolated polynucleotide encoding a chloroplast transit peptide; and an isolated polynucleotide encoding a full-length polypeptide having a sequence selected from the group consisting of SEQ ID NO:10 and SEQ ID NO:12; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length probable undecaprenyl pyrophosphate synthetase polypeptide having a sequence as set forth in SEQ ID NO:14; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves, and an isolated polynucleotide encoding a mitochondrial transit peptide; and an isolated polynucleotide encoding a full-length polypeptide which is a putative transcriptional regulator of fatty acid metabolism having a gntR-type HTH DNA-binding domain comprising amino acids 34 to 53 of SEQ ID NO:16; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide having a G3E, P-loop domain comprising a Walker A motif having a sequence as set forth in SEQ ID NO:99 and a GTP-specificity motif having a sequence as set forth in SEQ ID NO:100; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves, and an isolated polynucleotide encoding a full-length polypeptide which is a putative membrane protein having a sequence as set forth in SEQ ID NO:22; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length peroxisomal-coenzyme A synthetase polypeptide comprising an AMP-binding domain selected from the group consisting of amino acids 194 to 205 of SEQ ID NO:24, amino acids 202 to 213 of SEQ ID NO:26, amino acids 214 to 225 of SEQ ID NO:28, amino acids 195 to 206 of SEQ ID NO:30, amino acids 175 to 186 of SEQ ID NO:32, amino acids 171 to 182 of SEQ ID NO:34, amino acids 189 to 200 of SEQ ID NO:36, amino acids 201 to 212 of SEQ ID NO:38, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length histone H4 polypeptide having a G-A-K-R-H (SEQ ID NO:101) signature sequence domain selected from the group consisting of amino acids 3 to 92 of SEQ ID NO:40; amino acids 3 to 92 of SEQ ID NO:56; amino acids 3 to 92 of SEQ ID NO:42; and amino acids 3 to 92 of SEQ ID NO:44, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves or a constitutive promoter; an isolated polynucleotide encoding a chloroplast transit peptide; and an isolated polynucleotide encoding a full-length SYM1-type integral membrane polypeptide; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter capable of enhancing gene expression in leaves; an isolated polynucleotide encoding a mitochondrial transit peptide, and an isolated polynucleotide encoding a full-length vacuolar proton pump subunit H polypeptide, wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length F-ATPase subunit alpha polypeptide comprising an ATP synthase domain selected from the group consisting of amino acids 356 to 365 of SEQ ID NO:62; amino acids 254 to 263 of SEQ ID NO:64; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length F-ATPase subunit beta polypeptide comprising an ATP synthase domain selected from the group consisting of amino acids 353 to 362 of SEQ ID NO:66; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ABC transporter polypeptide having a sequence as set forth in SEQ ID NO:68; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a plastid transit peptide; and an isolated polynucleotide encoding a full-length photosystem I reaction center subunit psaK polypeptide having a psaGK signature comprising amino acids 56 to 73 of SEQ ID NO:70; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ferredoxin polypeptide comprising a Fer2 signature sequence selected from the group consisting of amino acids 11 to 87 of SEQ ID NO:72; amino acids 12 to 88 of SEQ ID NO:74; and amino acids 63 to 139 of SEQ ID NO:76, wherein the transgenic plant demonstrates increased yield as compared to a wild type plant of the same variety which does not comprise the expression cassette.

In another embodiment, the invention provides a transgenic plant transformed with an expression cassette comprising in operative association, an isolated polynucleotide encoding a promoter; an isolated polynucleotide encoding a plastid transit peptide; and an isolated polynucleotide encoding a full-length flavodoxin polypeptide having a Flavidoxin_{—}1 signature sequence comprising amino acids 6 to 160 of SEQ ID NO:78; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a plastid transit peptide; and an isolated polynucleotide encoding a full-length photosystem I reaction center subunit III psaF polypeptide comprising a PSI_PsaF signature sequence selected from the group consisting of amino acids 3 to 158 of SEQ ID NO:80; amino acids 43 to 217 of SEQ ID NO:82; amino acids 46 to 220 of SEQ ID NO:84; amino acids 50 to 224 of SEQ ID NO:86; and amino acids 50 to 224 of SEQ ID NO:88; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length cytochrome c553 (PetJ) polypeptide having aPSI_PsaF signature sequence comprising amino acids 38 to 116 of SEQ ID NO:90; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length photosystem II reaction center W (PsbW) polypeptide having a Cytochrome C signature sequence comprising amino acids 5 to 120 of SEQ ID NO:92; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter; an isolated polynucleotide encoding a plastid transit peptide; and an isolated polynucleotide encoding a full-length uroporphyrin-III c-methyltransferase (CobA) polypeptide having a sequence as set forth in SEQ ID NO:93; wherein the transgenic plant demonstrates increased yield as compared to a wild type plant of the same variety which does not comprise the expression cassette.

In another embodiment, the invention provides a transgenic plant transformed with an expression cassette comprising, in operative association, an isolated polynucleotide encoding a promoter and an isolated polynucleotide encoding a full-length precorrin-6b methylase having a Methyltransf_{—}12 signature sequence comprising amino acids 45 to 138 of SEQ ID NO:96; wherein the transgenic plant demonstrates increased yield as compared to a wild type plant of the same variety which does not comprise the expression cassette. The expression cassette of this embodiment may optionally comprise an isolated polynucleotide encoding a mitochondrial transit peptide.

In another embodiment, the invention provides a transgenic plant transformed with an expression cassette comprising, in operative association, an isolated polynucleotide encoding a promoter and an isolated polynucleotide encoding a decarboxylating precorrin-6y methylase having a TP_methylase signature sequence comprising amino acids 1 to 195 of SEQ ID NO:98; wherein the transgenic plant demonstrates increased yield as compared to a wild type plant of the same variety which does not comprise the expression cassette. The expression cassette of this embodiment may optionally comprise an isolated polynucleotide encoding a mitochondrial transit peptide.

In a further embodiment, the invention provides a seed produced by the transgenic plant of the invention, wherein the seed is true breeding for a transgene comprising the expression vectors described above. Plants derived from the seed of the invention demonstrate increased tolerance to an environmental stress, and/or increased plant growth, and/or increased yield, under normal or stress conditions as compared to a wild type variety of the plant.

In a still another aspect, the invention concerns products produced by or from the transgenic plants of the invention, their plant parts, or their seeds, such as a foodstuff, feedstuff, food supplement, feed supplement, fiber, cosmetic or pharmaceutical.

The invention further provides certain isolated polynucleotides identified in Table 1, and certain isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of the invention, and generating a transgenic plant from the plant cell, wherein the transgenic plant comprises the polynucleotide.

### BRIEF DESCRIPTION OF THE DRAWINGS

FIG. 1 shows an alignment of the amino acid sequences of the nucleotide binding domain containing proteins designated B2173 (SEQ ID NO:18), GM50181105 (SEQ ID NO:20). The alignment was generated using Align X of Vector NTI.

FIG. 2 shows an alignment of the amino acid sequences of the peroxisomal-coenzyme A synthetases designated YBR222C (SEQ ID NO:24), BN51408632 (SEQ ID NO:26), BN51423788 (SEQ ID NO:28), BN51486050 (SEQ ID NO:30), GM50942269 (SEQ ID NO:32), GM59534234 (SEQ ID NO:34), GM59654631 (SEQ ID NO:36), GM59778298 (SEQ ID NO:38). The alignment was generated using Align X of Vector NTI.

FIG. 3 shows an alignment of the amino acid sequences of the histone H4 designated YNL030W (SEQ ID NO:40), GM53663330 (SEQ ID NO:56), LU62237699 (SEQ ID NO:42), OS36075085 (SEQ ID NO:44). The alignment was generated using Align X of Vector NTI.

FIG. 4 shows an alignment of the amino acid sequences of the SYM1-type integral membrane proteins designated YLR251W (SEQ ID NO:62), BN42108421 (SEQ ID NO:64), GMsf23a01 (SEQ ID NO:50), HV62697288 (SEQ ID NO:52), LU61649286 (SEQ ID NO:54), OS40298410 (SEQ ID NO:56). The alignment was generated using Align X of Vector NTI.

FIG. 5 shows an alignment of the amino acid sequences of the V-ATPase subunit H polypeptides designated YPR036W (SEQ ID NO:58), BN51362135 (SEQ ID NO:60). The alignment was generated using Align X of Vector NTI.

FIG. 6 shows an alignment of the amino acid sequences of the F-ATPase subunit alphas designated SLL1326 (SEQ ID NO:62), LU61815688 (SEQ ID NO:64). The alignment was generated using Align X of Vector NTI.

FIG. 7 shows an alignment of the amino acid sequences of the ferredoxins designated sll1382 (SEQ ID NO:72), BN42448747 (SEQ ID NO:74), GM49779037 (SEQ ID NO:76). The alignment was generated using Align X of Vector NTI.

FIG. 8 shows an alignment of the amino acid sequences of the photosystem I reaction center subunit III proteins designated sll0819 (SEQ ID NO:80), BN51362302 (SEQ ID NO:82), BNDLM1779_{—}30 (SEQ ID NO:84), GMsk95f02 (SEQ ID NO:86), and GMso56a01 (SEQ ID NO:88). The alignment was generated using Align X of Vector NTI.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, is an improved yield in accordance with the invention.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli; and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are A. thaliana, Nicotiana tabacum, rice, oilseed rape, canola, soybean, corn (maize), cotton, and wheat.

### A. Untargeted Uncharacterized Proteins

In one embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide having a sequence selected from the group consisting of SEQ ID NO:2, SEQ ID NO:4; SEQ ID NO:6; and SEQ ID NO:8; wherein the transgenic plant demonstrates increased yield as compared to a wild type plant of the same variety which does not comprise the expression cassette.

### B. Plastid-Targeted Unknown Proteins

In another embodiment, the invention provides a transgenic plant transformed with an expression cassette comprising, in operative association, an isolated polynucleotide encoding a promoter capable of enhancing gene expression in leaves; and an isolated polynucleotide encoding a chloroplast transit peptide; and an isolated polynucleotide encoding a full-length polypeptide having a sequence selected from the group consisting of SEQ ID NO:10 and SEQ ID NO:12; wherein the transgenic plant demonstrates increased yield as compared to a wild type plant of the same variety which does not comprise the expression cassette.

### C. Undecaprenyl Pyrophosphate Synthetase

In another embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length polypeptide having a sequence as set forth in SEQ ID NO:14; wherein the transgenic plant demonstrates increased yield as compared to a wild type plant of the same variety which does not comprise the expression cassette.

### D. Putative Transcriptional Regulator of Fatty Acid Metabolism

In another embodiment, the invention provides a transgenic plant transformed with an expression cassette comprising, in operative association, an isolated polynucleotide encoding a promoter capable of enhancing gene expression in leaves, and an isolated polynucleotide encoding a mitochondrial transit peptide; and an isolated polynucleotide encoding a full-length polypeptide which is a putative transcriptional regulator of fatty acid metabolism, wherein the transgenic plant demonstrates increased yield as compared to a wild type plant of the same variety which does not comprise the expression cassette. Gene B1187 (SEQ ID NO:15) encodes a putative transcriptional regulator of fatty acid metabolism. Transcriptional regulators are characterized, in part, by the type and context of their DNA-binding domains. The gntR-type HTH DNA-binding domain characterizes, in part, the class of transcriptional regulators of fatty acid metabolism exemplified by the B1187 protein (SEQ ID NO:16).

The transgenic plant of this embodiment may comprise any polynucleotide encoding a putative transcriptional regulator of fatty acid metabolism. Preferably, the transgenic plant of this embodiment comprises a polynucleotide encoding a full-length polypeptide, wherein the polypeptide comprises a gntR-type HTH DNA-binding domain. Preferably, the polynucleotide encodes a transcriptional regulator of fatty acid metabolism polypeptide comprising a gntR-type HTH DNA-binding domain, wherein the domain has a sequence consisting of amino acids 34 to 53 of SEQ ID NO:16. More preferably, the polynucleotide encodes a transcriptional regulator of fatty acid metabolism polypeptide comprising a transcriptional regulator domain consisting of amino acids 3 to 90 of SEQ ID NO:16. Most preferably, the polynucleotide encodes a putative transcriptional regulator of fatty acid metabolism polypeptide comprising amino acids 1 to 239 of SEQ ID NO: 4.

### E. G3E-Family, P-Loop Domain, Nucleotide-Binding Proteins

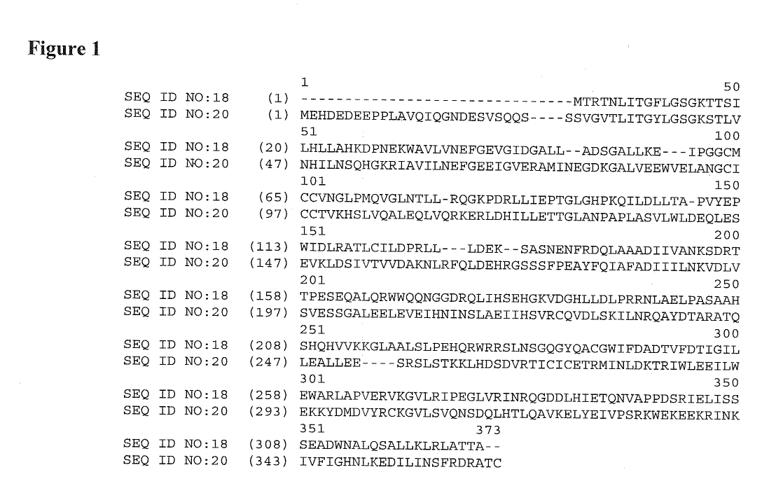

In another embodiment, the invention provides a transgenic plant transformed with an expression cassette comprising, in operative association, an isolated polynucleotide encoding a promoter capable of enhancing gene expression in leaves; and an isolated polynucleotide encoding a full-length polypeptide which is a nucleotide binding domain containing polypeptide; wherein the transgenic plant demonstrates increased yield as compared to a wild type plant of the same variety which does not comprise the expression cassette. Gene B2173 (SEQ ID NO:17) encodes a G3E family P-loop GTPase domain-containing polypeptide (SEQ ID NO:18). G3E family P-loop GTPase domains are characterized, in part, by the presence of two distinctive motifs, a Walker A motif near the N-terminus of the mature polypeptide and a GTP-specificity motif. The Walker A motif is G-x-x-x-x-G-K-S/T (SEQ ID NO:99). The Walker A motif functions to position the triphosphate moiety of a bound nucleotide. The GTP-specificity motif is an amino acid stretch of NIT-K-x-D (SEQ ID NO:100) and is thought to be essential for the specificity for guanine over other bases. Such conserved motifs are exemplified in the proteins set forth in FIG. 1.

The transgenic plant of this embodiment may comprise any polynucleotide encoding a G3E family P-loop GTPase domain nucleotide-binding protein. Preferably, the transgenic plant of this embodiment comprises a polynucleotide encoding a full-length polypeptide having nucleotide-binding activity, wherein the polypeptide comprises a domain comprising a Walker A motif combined with a GTP-specificity motif, wherein the Walker A motif has a sequence selected from the group consisting of amino acids 9 to 16 of SEQ ID NO:18, amino acids 36 to 43 of SEQ ID NO:20 and the GTP-specificity motif has a sequence selected from the group consisting of amino acids 152 to 155 of SEQ ID NO:18, amino acids 191 to 191 of SEQ ID NO:20. More preferably, the polynucleotide encodes a full-length polypeptide having nucleotide-binding activity, wherein the polypeptide comprises a domain selected from the group consisting of amino acids 6 to 320 of SEQ ID NO:18, amino acids 33 to 355 of SEQ ID NO:20. Most preferably, the transgenic plant of this embodiment comprises a polynucleotide encoding a nucleotide-binding protein comprising amino acids 1 to 328 of SEQ ID NO:18; amino acids 1 to 365 of SEQ ID NO:20.

### F. Putative Membrane Protein

In another embodiment, the invention provides a transgenic plant transformed with an expression cassette comprising, in operative association, an isolated polynucleotide encoding a promoter capable of enhancing gene expression in leaves; and an isolated polynucleotide encoding a full-length putative membrane polypeptide having a sequence as set forth in SEQ ID NO:22; wherein the transgenic plant demonstrates increased yield as compared to a wild type plant of the same variety which does not comprise the expression cassette. Gene B2670 (SEQ ID NO:21) encodes a putative membrane protein (SEQ ID NO:22). The transgenic plant of this embodiment may comprise any polynucleotide encoding a putative membrane protein having a sequence comprising amino acids 1 to 149 of SEQ ID NO:22.

### G. Peroxisomal-Coenzyme A Synthetases

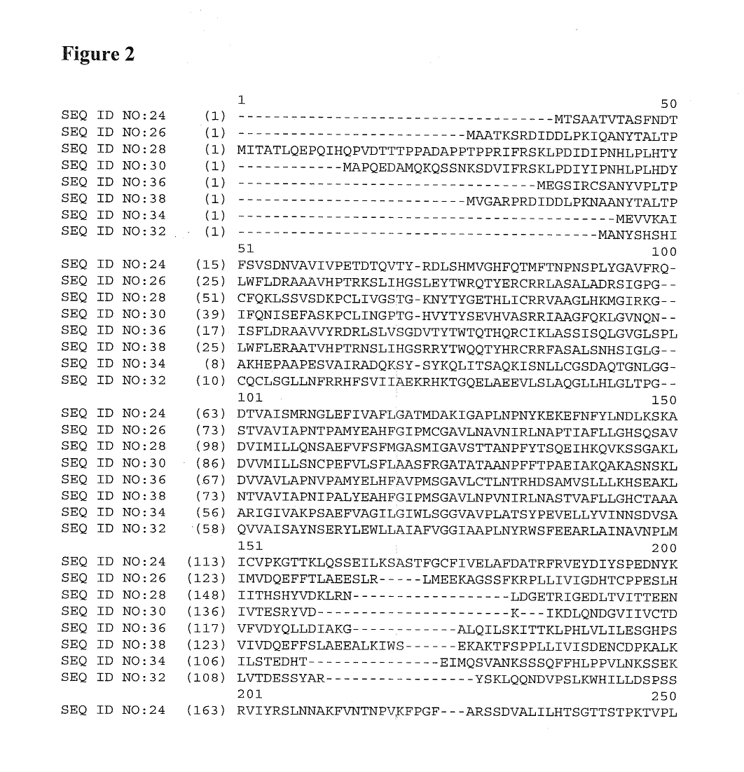

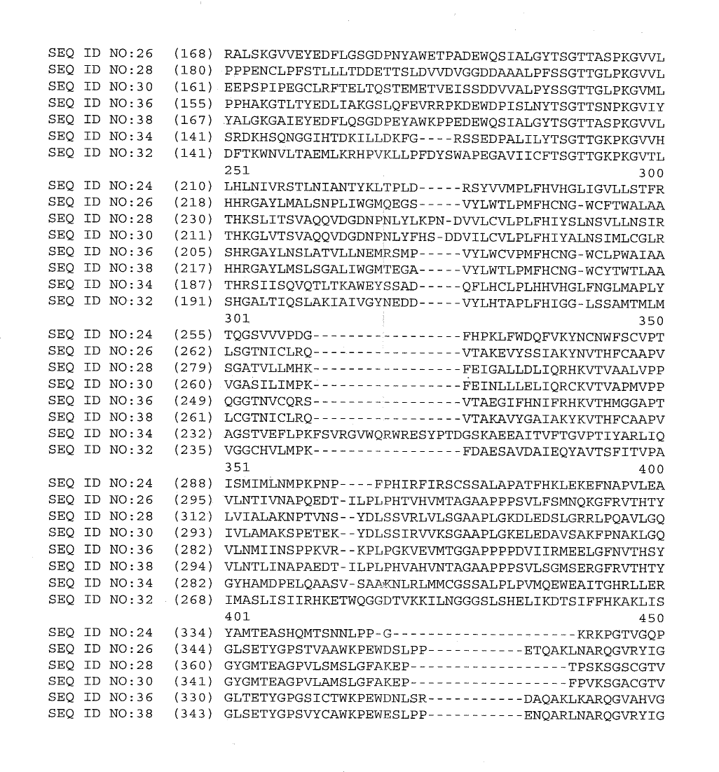

In another embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length peroxisomal-coenzyme A synthetase polypeptide; wherein the transgenic plant demonstrates increased yield as compared to a wild type plant of the same variety which does not comprise the expression cassette. Gene YBR222C (SEQ ID NO:23) encodes a peroxisomal-coenzyme A synthetase protein (SEQ ID NO:24). Peroxisomal-coenzyme A synthetases are characterized, in part, by the presence of an AMP-binding domain which has a distinctive signature sequence. Such conserved signature sequences are exemplified in the peroxisomal-coenzyme A synthetase proteins set forth in FIG. 2.

The transgenic plant of this embodiment may comprise any polynucleotide encoding a peroxisomal-coenzyme A synthetase protein. Preferably, the transgenic plant of this embodiment comprises a polynucleotide encoding a full-length polypeptide having peroxisomal-coenzyme A synthetase activity, wherein the polypeptide comprises an AMP-binding domain having a sequence selected from the group consisting of amino acids 194 to 205 of SEQ ID NO:24, amino acids 202 to 213 of SEQ ID NO:26, amino acids 214 to 225 of SEQ ID NO:28, amino acids 195 to 206 of SEQ ID NO:30, amino acids 175 to 186 of SEQ ID NO:32, amino acids 171 to 182 of SEQ ID NO:34, amino acids 189 to 200 of SEQ ID NO:36, amino acids 201 to 212 of SEQ ID NO:38. More preferably, the polynucleotide encodes a full-length polypeptide having peroxisomal-coenzyme A synthetase activity, wherein the polypeptide comprises a domain selected from the group consisting of amino acids 198 to 456 of SEQ ID NO:24, amino acids 206 to 477 of SEQ ID NO:26, amino acids 218 to 487 of SEQ ID NO:28, amino acids 199 to 468 of SEQ ID NO:30, amino acids 179 to 457 of SEQ ID NO:32, amino acids 175 to 452 of SEQ ID NO:34, amino acids 193 to 463 of SEQ ID NO:36, amino acids 205 to 476 of SEQ ID NO:38. Most preferably, the transgenic plant of this embodiment comprises a polynucleotide encoding a peroxisomal-coenzyme A synthetase comprising amino acids 1 to 543 of SEQ ID NO:24, amino acids 1 to 569 of SEQ ID NO:26, amino acids 1 to 565 of SEQ ID NO:28, amino acids 1 to 551 of SEQ ID NO:30, amino acids 1 to 560 of SEQ ID NO:32, amino acids 1 to 543 of SEQ ID NO:34, amino acids 1 to 553 of SEQ ID NO:36, amino acids 1 to 568 of SEQ ID NO:38.

### H. Histone H4 Proteins

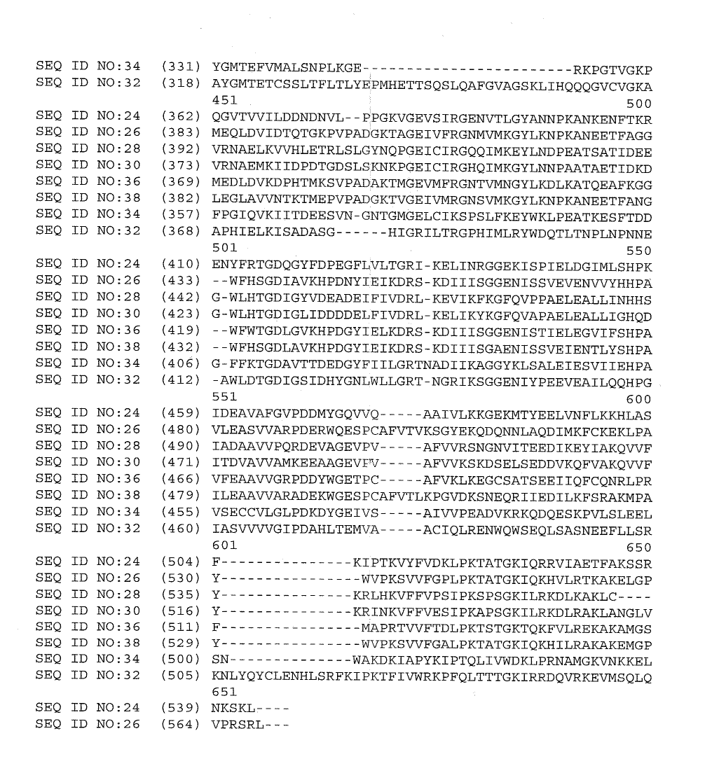

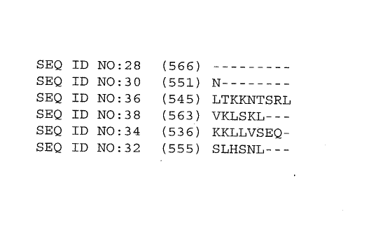

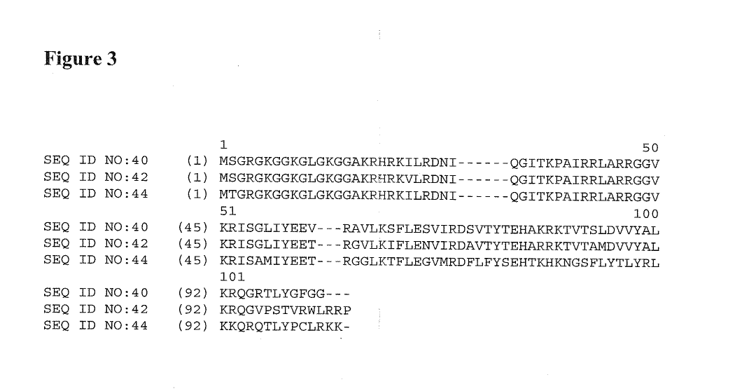

In another embodiment, the invention provides a transgenic plant transformed with an expression cassette comprising, in operative association, an isolated polynucleotide encoding a promoter capable of enhancing gene expression in leaves; and an isolated polynucleotide encoding a mitochondrial transit peptide; and an isolated polynucleotide encoding a full-length histone H4 polypeptide; wherein the transgenic plant demonstrates increased yield as compared to a wild type plant of the same variety which does not comprise the expression cassette. Gene YNL030W (SEQ ID NO:39) encodes a histone H4 protein (SEQ ID NO:40). Histones are not naturally found in mitochondria, although histone-like proteins have been found. Together with the other core histones, H4 histones form the histone octamer around which nuclear DNA is wrapped in the formation of nucleosomes, the primary structural units of chromatin. Histone H4 proteins are characterized, in part, by the presence of the distinctive signature sequence, G-A-K-R-H (SEQ ID NO:101), which is located between positions 14 and 18 of the protein. This conserved signature sequence is exemplified in the histone H4 proteins set forth in FIG. 3.

The transgenic plant of this embodiment may comprise any polynucleotide encoding a histone H4 protein. Preferably, the transgenic plant of this embodiment comprises a polynucleotide encoding a full-length polypeptide having histone H4 synthetase activity, wherein the polypeptide comprises a domain comprising a histone H4 signature having a sequence selected from the group consisting of amino acids 15 to 19 of SEQ ID NO:40, amino acids 15 to 19 of SEQ ID NO:56, amino acids 15 to 19 of SEQ ID NO:42, amino acids 15 to 19 of SEQ ID NO:44. More preferably, the polynucleotide encodes a full-length polypeptide having histone H4 activity, wherein the polypeptide comprises a domain selected from the group consisting of amino acids amino acids 3 to 92 of SEQ ID NO:40, amino acids 3 to 92 of SEQ ID NO:56, amino acids 3 to 92 of SEQ ID NO:42, amino acids 3 to 92 of SEQ ID NO:44. Most preferably, the transgenic plant of this embodiment comprises a polynucleotide encoding a histone H4 comprising amino acids 1 to 103 of SEQ ID NO:40, amino acids 1 to 103 of SEQ ID NO:56, amino acids 1 to 106 of SEQ ID NO:42, amino acids 1 to 105 of SEQ ID NO:44.

### I. SYM1-Type Integral Membrane Proteins

In another embodiment, the invention provides a transgenic plant transformed with an expression cassette comprising, in operative association, an isolated polynucleotide encoding a promoter capable of enhancing gene expression in leaves; an isolated polynucleotide encoding a chloroplast transit peptide; and polynucleotide encoding a full-length SYM1-type integral membrane protein, wherein the transgenic plant demonstrates increased yield as compared to a wild type plant of the same variety which does not comprise the expression cassette.

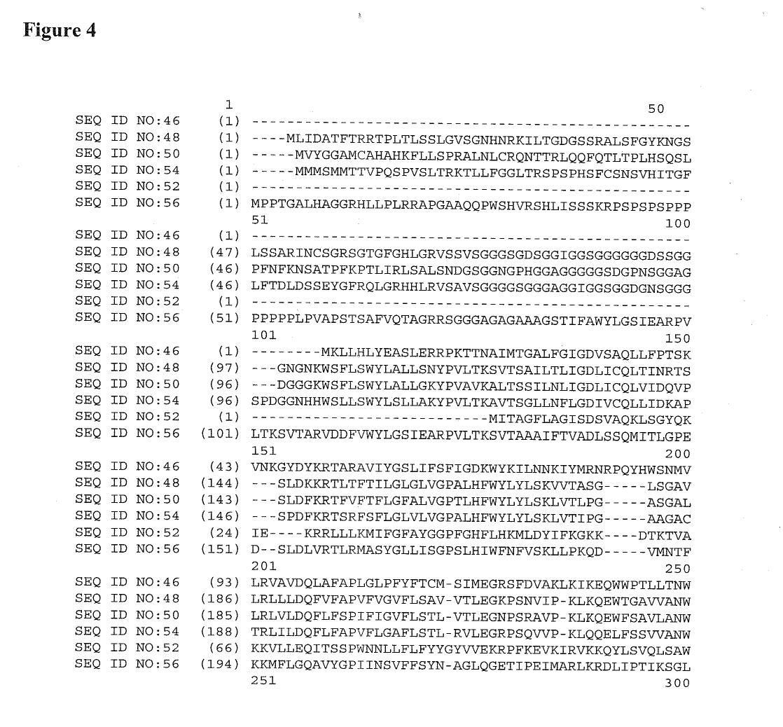

Gene YLR251W (SEQ ID NO: 61) is SYM1 (for “Stress-inducible Yeast Mpv17”). Sym1 is an integral membrane protein that has an important role in membrane transport during heat shock. Example 2 below shows that expression of gene YLR251W (SEQ ID NO:61) under control of the USP promoter or the PCUbi promoter and targeted to the chloroplast, results in larger plants either under water limiting growth conditions or when well-watered. FIG. 4 shows an alignment of representative SYM1-type polypeptides which may be employed in accordance with this embodiment of the invention.

The transgenic plant of this embodiment may comprise any polynucleotide encoding a SYM1-type integral membrane polypeptide. Preferably, the transgenic plant of this embodiment comprises a polynucleotide encoding a full-length SYM1-type integral membrane polypeptide, wherein the polypeptide comprises a domain selected from the group consisting of amino acids 31 to 171 of SEQ ID NO:62; amino acids 132 to 263 of SEQ ID NO:64; amino acids 131 to 262 of SEQ ID NO:50; amino acids 12 to 145 of SEQ ID NO:52; amino acids 134 to 265 of SEQ ID NO:54; and amino acids 139 to 272 of SEQ ID NO:56. Most preferably, the transgenic plant of this embodiment comprises a polynucleotide encoding a SYM1-type integral membrane polypeptide having a sequence comprising amino acids 1 to 197 of SEQ ID NO:62; amino acids 1 to 278 of SEQ ID NO:64; amino acids 1 to 277 of SEQ ID NO:50; amino acids 1 to 161 of SEQ ID NO:52; amino acids 1 to 280 of SEQ ID NO:54; or amino acids 1 to 293 of SEQ ID NO:56.

J. Vacuolar Pump Subunit H polypeptides

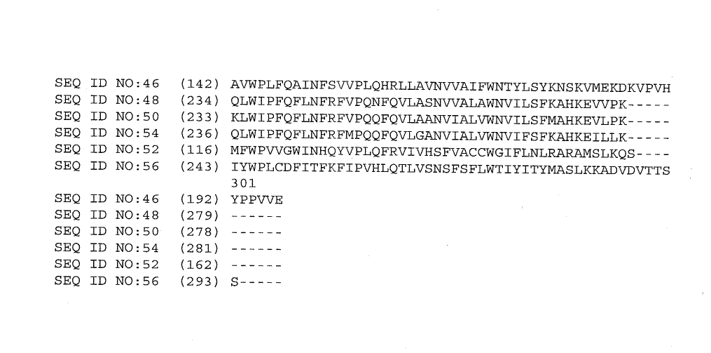

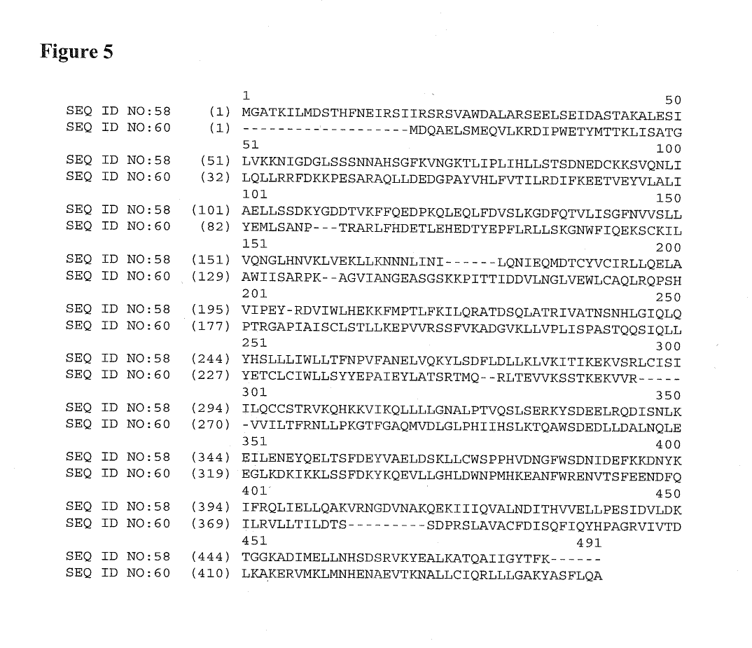

In another embodiment, the invention provides a transgenic plant transformed with an expression cassette comprising, in operative association, an isolated polynucleotide encoding a promoter capable of enhancing gene expression in leaves, an isolated polynucleotide encoding a mitochondrial transit peptide, and an isolated polynucleotide encoding a full-length vacuolar proton pump subunit H polypeptide, wherein the transgenic plant demonstrates increased yield as compared to a wild type plant of the same variety which does not comprise the expression cassette. Gene YPRO36W (SEQ ID NO:57) encodes V-type ATPase subunit H, which is a regulatory subunit necessary for the activity, but not the assembly, of V-type ATPases in yeast. Example 2 below shows that expression of gene YPR036W (SEQ ID NO: 73) under control of the USP promoter and targeted to the mitochondria results in larger plants under water limiting growth conditions. FIG. 5 shows an alignment of representative V-type ATPase subunit H polypeptides which may be employed in accordance with this embodiment of the invention.

The transgenic plant of this embodiment may comprise any polynucleotide encoding a V-type ATPase subunit H polypeptide. Preferably, the transgenic plant of this embodiment comprises a polynucleotide encoding a full-length polypeptide having V-type ATPase subunit H activity, wherein the polypeptide comprises a domain which has a sequence selected from the group consisting of amino acids 38 to 470 of SEQ ID NO:58; amino acids 19 to 436 of SEQ ID NO:60. Most preferably, the polynucleotide encodes a V-type ATPase subunit H polypeptide comprising amino acids 1 to 478 of SEQ ID NO:58; amino acids 1 to 450 of SEQ ID NO:60.

K. F-ATPase Subunit Alpha Polypeptides

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a F-ATPase subunit alpha polypeptide; wherein the transgenic plant demonstrates increased yield as compared to a wild type plant of the same variety which does not comprise the expression cassette. Gene SLL1326 (SEQ ID NO:61) encodes F-ATPase subunit alpha, which is an essential component of the F-ATP holoenzyme. Example 2 below shows that expression of gene SLL1326 (SEQ ID NO:61) under control of the ubiquitin promoter and targeted to the mitochondria results in larger plants under water limiting growth conditions.

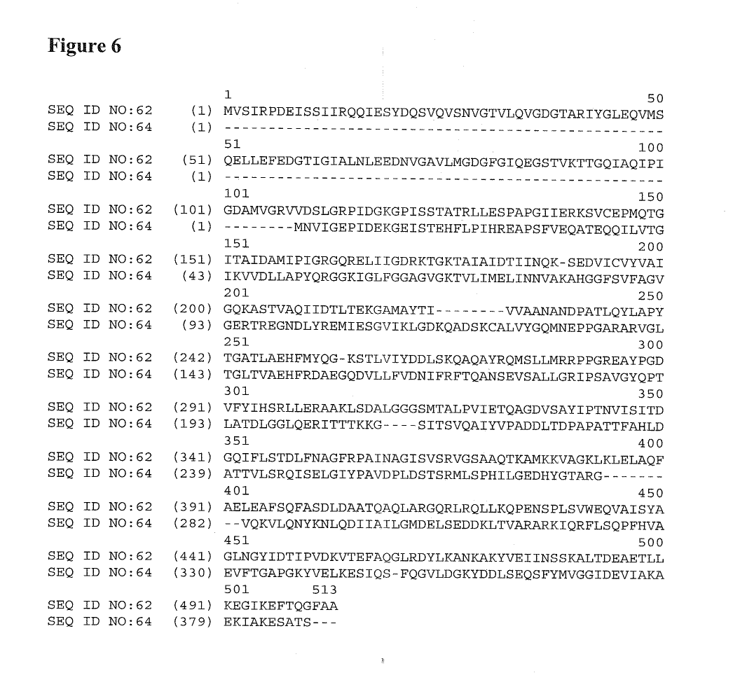

F-ATPases are the prime producers of ATP, using the proton gradient generated by oxidative phosphorylation in mitochondria or photosynthesis in chloroplasts. Both the alpha and the beta subunits of F-ATPases comprise an ATP synthase domain which is characterized by a distinctive signature sequence with the sequence “P-[SAP]-[LIV]-[DNH]-{LKGN}-{F}-{S}-S-{DCPH}-S” where amino acid positions within square brackets can be any of the designated residues, amino acid positions within curly brackets can be any amino acid residue except the one(s) listed and unbracketed amino acid positions can only be that specific amino acid residue. Such conserved signature seqeunces are exemplified in the F-ATPase subunit alpha proteins set forth in FIG. 6.

The transgenic plant of this embodiment may comprise any polynucleotide encoding a F-ATPase subunit alpha. Preferably, the transgenic plant of this embodiment comprises a polynucleotide encoding a full-length polypeptide having F-ATPase subunit alpha activity, wherein the polypeptide comprises a domain comprising an ATP synthase signature sequence selected from the group consisting of amino acids 356 to 365 of SEQ ID NO:62; amino acids 254 to 263 of SEQ ID NO:64. More preferably, the polynucleotide encodes a full-length polypeptide having F-ATPase subunit alpha activity, wherein the polypeptide comprises a domain selected from the group consisting of amino acids 149 to 365 of SEQ ID NO:62; amino acids 41 to 263 of SEQ ID NO:64. Most preferably, the transgenic plant of this embodiment comprises a polynucleotide encoding an F-ATPase subunit alpha comprising amino acids 1 to 503 of SEQ ID NO:62; amino acids 1 to 388 of SEQ ID NO:64.

### L. F-ATPase Subunit Beta Polypeptides

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length F-ATPase subunit beta polypeptide; wherein the transgenic plant demonstrates increased yield as compared to a wild type plant of the same variety which does not comprise the expression cassette. Gene SLR1329 (SEQ ID NO:65) encodes F-ATPase subunit beta, which like the alpha subunit is an essential component of the F-ATP holoenzyme. Example 2 below shows that expression of gene Gene SLR1329 (SEQ ID NO:65) under control of the ubiquitin promoter and targeted to the mitochondria results in larger plants under water limiting growth conditions. F-ATPase subunit beta enzymes, are also characterized, in part, by the presence of the ATP synthase signature sequence “P-[SAP]-[LIV]-[DNH]-{LKGN}-{F}-{S}-S-{DCPH}-S” as described for the alpha subunits. Such conserved motifs are exemplified in the F-ATPase subunit beta proteins set forth in FIG. 6.

The transgenic plant of this embodiment may comprise any polynucleotide encoding a F-ATPase subunit beta. Preferably, the transgenic plant of this embodiment comprises a polynucleotide encoding a full-length polypeptide having F-ATPase subunit beta activity, wherein the polypeptide comprises polynucleotide encoding an F-ATPase subunit beta comprising amino acids 1 to 483 of SEQ ID NO:66.

### M. ABC Transporters

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ABC transporter polypeptide; wherein the transgenic plant demonstrates increased yield as compared to a wild type plant of the same variety which does not comprise the expression cassette. Gene SLR0977 (SEQ ID NO:67) encodes an ABC transporter, which are membrane spanning proteins that utilize the energy of ATP hydrolysis to transport a wide variety of substrates across membranes. Example 2 below shows that expression of gene SLR0977 (SEQ ID NO:67) under control of the ubiquitin promoter and targeted to the mitochondria results in larger plants under water limiting growth conditions.

The transgenic plant of this embodiment may comprise any polynucleotide encoding an ABC transporter. Most preferably, the transgenic plant of this embodiment comprises a polynucleotide encoding an ABC transporter comprising amino acids 1 to 276 of SEQ ID NO:68.

N. PS-I Subunit psaK Polypeptides

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length PS-I subunit psaK polypeptide, wherein the transgenic plant demonstrates increased yield as compared to a wild type plant of the same variety which does not comprise the expression cassette. As demonstrated in Example 2 below, transgenic Arabidopsis plants containing the Synechocystis sp. gene ssr0390 (SEQ ID NO:69) targeted to the chloroplast demonstrate increased biomass as compared to control Arabidopsis plants. The ssr0390 gene encodes a psaK subunit of PS-I, which is characterized, in part, by the presence of a distinctive PsaGK signature sequence representative of the psaG/psaK family of genes. The photosystem I psaGK signature sequence is [GTND]-[FPMI]-x-[LIVMH]-x-[DEAT]-x(2)-[GA]-x-[GTAM]-[STA]-x-G-H-x-[LIVM]-[GAS] where amino acid positions within square brackets can be any of the designated residues. The protein, psaK, is a small hydrophobic protein with two transmembrane domains (amino acids 14 to 34 and amino acids 61 to 81 of SEQ ID NO:70) related to psaG in plants. The psaGK signature sequence is found at residue positions 56 to 73 and thus resides almost completely within the second transmembrane domain.

The transgenic plant of this embodiment may comprise any polynucleotide encoding a full-length psaK subunit. Preferably, the transgenic plant of this embodiment comprises a polynucleotide encoding a full-length polypeptide having psaK activity, wherein the polypeptide comprises a PSI_PsaK signature comprising amino acids 14 to 86 of SEQ ID NO:2. More preferably, the transgenic plant of this embodiment comprises a polynucleotide encoding a photosystem I reaction center psaK subunit having a sequence comprising amino acids 1 to 86 of SEQ ID NO:2.

### O. Ferredoxins

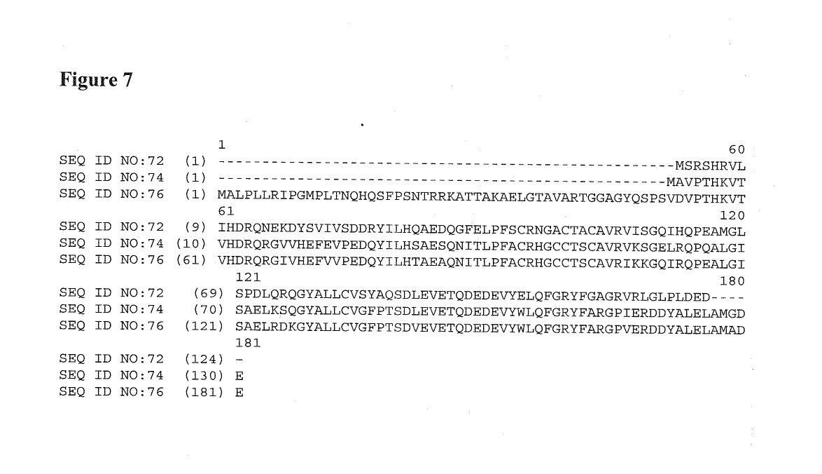

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length ferredoxin polypeptide, wherein the transgenic plant demonstrates increased yield as compared to a wild type plant of the same variety which does not comprise the expression cassette. As demonstrated in Example 2 below, transgenic Arabidopsis plants containing the Synechocystis sp. gene sll1382 (SEQ ID NO:71) targeted to mitochondria demonstrate increased biomass as compared to control Arabidopsis plants. The sll1382 gene encodes ferredoxin (PetF), characterized, in part, by the presence of a Fer2 signature sequence. Such signature sequences are exemplified in the ferredoxin proteins set forth in FIG. 7.

The transgenic plant of this embodiment may comprise any polynucleotide encoding a ferredoxin. Preferably, the transgenic plant of this embodiment comprises a polynucleotide encoding a full-length polypeptide having ferredoxin activity, wherein the polypeptide comprises a Fer2 signature sequence selected from the group consisting of amino acids 11 to 87 of SEQ ID NO:72; amino acids 12 to 88 of SEQ ID NO:74; amino acids 63 to 139 of SEQ ID NO:76. More preferably, the transgenic plant of this embodiment comprises a polynucleotide encoding a ferredoxin polypeptide having a sequence comprising amino acids 1 to 122 of SEQ ID NO:72; amino acids 1 to 128 of SEQ ID NO:74; amino acids 1 to 179 of SEQ ID NO:76.

### P. Flavodoxins

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length flavodoxin polypeptide comprising amino acids 6 to 160 of SEQ ID NO:78, wherein the transgenic plant demonstrates increased yield as compared to a wild type plant of the same variety which does not comprise the expression cassette. As demonstrated in Example 2 below, transgenic Arabidopsis plants containing the Synechocystis sp. gene sll0248 (SEQ ID NO:77) targeted to the chloroplast demonstrate increased biomass as compared to control Arabidopsis plants. The sll0248 gene encodes flavodoxin and is characterized, in part, by the presence of the Flavodoxin_{—}1 signature sequence represented as amino acids 6 to 160 of SEQ ID NO:78.

The transgenic plant of this embodiment may comprise any polynucleotide encoding a full-length flavodoxin polypeptide comprising amino acids 6 to 160 of SEQ ID NO:78. Preferably, the transgenic plant of this embodiment comprises a polynucleotide encoding a full-length flavodoxin having a sequence comprising amino acids 1 to 170 of SEQ ID NO:78.

Q. PS-I psaF Polypeptides

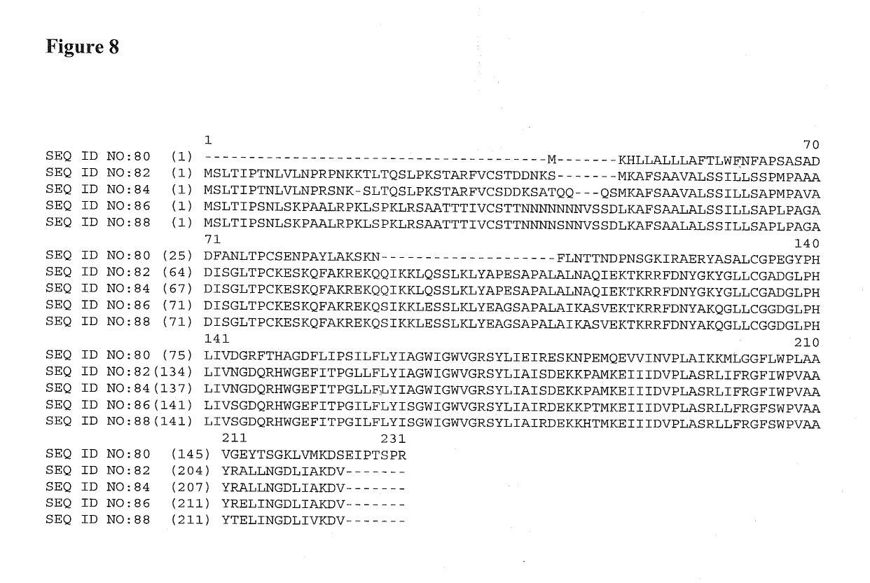

In another embodiment, the invention provides a transgenic plant transformed with an expression cassette comprising, in operative association an isolated polynucleotide encoding a promoter; an isolated polynucleotide encoding a chloroplast transit peptide; and an isolated polynucleotide encoding a full-length PS-I psaF polypeptide, wherein the transgenic plant demonstrates increased yield as compared to a wild type plant of the same variety which does not comprise the expression cassette. As demonstrated in Example 2 below, transgenic Arabidopsis plants containing the Synechocystis sp. gene sll0819 (SEQ ID NO:79) targeted to the chloroplast demonstrate increased biomass as compared to control Arabidopsis plants. The sll0819 gene encodes PS-I subunit III (PsaF) characterized, in part, by the presence of a PSI_PsaF signature sequence. Such signature sequences are exemplified in the PS-I subunit III proteins set forth in FIG. 8.

The transgenic plant of this embodiment may comprise any polynucleotide encoding a PS-I subunit III. Preferably, the transgenic plant of this embodiment comprises a polynucleotide encoding a full-length polypeptide having PS-I subunit III activity, wherein the polypeptide comprises a PSI_PsaF signature sequence selected from the group consisting of amino acids 3 to 158 of SEQ ID NO:80; amino acids 43 to 217 of SEQ ID NO:82; amino acids 46 to 220 of SEQ ID NO:84; amino acids 50 to 224 of SEQ ID NO:86; and amino acids 50 to 224 of SEQ ID NO:88. More preferably, the transgenic plant of this embodiment comprises a polynucleotide encoding a plant PS-I subunit III having a sequence comprising amino acids 1 to 217 of SEQ ID NO:82; amino acids 1 to 220 of SEQ ID NO:84; amino acids 1 to 224 of SEQ ID NO:86; or amino acids 1 to 224 of SEQ ID NO:88.

R. Cytochrome c553 Proteins

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length cytochrome c553 (petJ) polypeptide, wherein the transgenic plant demonstrates increased biomass as compared to a wild type plant of the same variety which does not comprise the expression cassette. As demonstrated in Example 2 below, transgenic Arabidopsis plants containing the Synechocystis sp. gene sll1796 (SEQ ID NO:89) targeted to mitochondria demonstrate increased yield as compared to control Arabidopsis plants.

Gene sll1796 (SEQ ID NO:89) encodes cytochrome C553. Cytochrome C553 (PetJ), also known as cytochrome c6, is involved in photosynthetic electron transport. PetJ functions as an electron carrier between membrane-bound cytochrome b6-f and photosystem I, which is a function conducted by plastocyanin in higher plants. Photosynthetic electron transport from the cytochrome bf complex to PS-I can be mediated by cytochrome c6 or plastocyanin, depending on the concentration of copper in the growth medium. Cytochrome c553 protiens are characterized, in part, by the presence of a Cytochrom_C signature sequence represented as amino acids 38 to 116 of SEQ ID NO:90. The transgenic plant of this embodiment may comprise any polynucleotide encoding a cytochrome c553 protein. Preferably, the transgenic plant of this embodiment comprises a polynucleotide encoding a full-length polypeptide having cytochrome c553 activity, wherein the polypeptide comprises a Cytochrom_C signature sequence comprising amino acids 38 to 116 of SEQ ID NO:90. More preferably, the transgenic plant of this embodiment comprises a polynucleotide encoding a cytochrome c553 polypeptide having a sequence comprising amino acids 1 to 120 of SEQ ID NO:90.

### S. PS_II W Polypeptides

In another embodiment, the invention provides a transgenic plant transformed with an expression cassette comprising, in operative association, an isolated polynucleotide encoding a promoter; an isolated polynucleotide encoding a mitochondrial transit peptide; and an isolated polynucleotide encoding a full-length PS-II W (PsbW) polypeptide, wherein the transgenic plant demonstrates increased yield as compared to a wild type plant of the same variety which does not comprise the expression cassette. As demonstrated in Example 2 below, transgenic Arabidopsis plants containing the Synechocystis sp. gene sll1739 (SEQ ID NO:91) targeted to mitochondria demonstrate increased biomass as compared to control Arabidopsis plants. Gene slr1739 (SEQ ID NO:91) encodes psbW, which is characterized, in part, by the presence of the PsbW signature sequence represented as amino acids 5 to 120 of SEQ ID NO:92.

The transgenic plant of this embodiment may comprise any polynucleotide encoding a full-length PsbW protein comprising a PsbW signature sequence comprising amino acids 5 to 120 of SEQ ID NO:92. Most preferably, the transgenic plant of this embodiment comprises a polynucleotide encoding a PsbW activity having a sequence comprising amino acids 1 to 122 of SEQ ID NO:92.

### T. Uroporphyrin-III C-Methyltransferases

In another embodiment, the invention provides a transgenic plant transformed with an expression cassette comprising, in operative association, an isolated polynucleotide encoding a promoter; an isolated polynucleotide encoding a chloroplast transit peptide; and an isolated polynucleotide encoding a full-length uroporphyrin-III c-methyltransferase (CobA) polypeptide, wherein the transgenic plant demonstrates increased biomass as compared to a wild type plant of the same variety which does not comprise the expression cassette. As demonstrated in Example 2 below, transgenic Arabidopsis plants containing the Synechocystis sp. gene sll0378 (SEQ ID NO:93) targeted to chloroplast demonstrate increased yield as compared to control Arabidopsis plants. Gene sll0378 (SEQ ID NO:93) encodes uroporphyrin-III C-methyltransferase (CobA). Uroporphyrin-III c-methyltransferases are characterized, in part, by the presence of a TP_methylase signature sequence.

The transgenic plant of this embodiment may comprise any plant polynucleotide encoding a uroporphyrin-III c-methyltransferase. Preferably, the transgenic plant of this embodiment comprises a polynucleotide encoding a full-length polypeptide having uroporphyrin-III c-methyltransferase activity, having a sequence comprising amino acids 1 to 263 of SEQ ID NO:94.

### U. Precorrin-6b Methylases

In another embodiment, the invention provides a transgenic plant transformed with an expression cassette comprising, in operative association an isolated polynucleotide encoding a promoter and an isolated polynucleotide encoding a full-length precorrin-6b methylase polypeptide, wherein the transgenic plant demonstrates increased yield as compared to a wild type plant of the same variety which does not comprise the expression cassette. The expression cassette of this embodiment may optionally comprise an isolated polynucleotide encoding a mitochondrial transit peptide. As demonstrated in Example 2 below, transgenic Arabidopsis plants containing the Synechocystis sp. gene slr1368 (SEQ ID NO:95) demonstrate increased biomass as compared to control Arabidopsis plants. Gene slr1368 encodes a precorrin-6b methylase characterized, in part, by the presence of a Methyltransf_{—}12 signature sequence.

The transgenic plant of this embodiment may comprise any polynucleotide encoding a precorrin-6b methylase. Preferably, the transgenic plant of this embodiment comprises a polynucleotide encoding a full-length polypeptide having precorrin-6b methylase activity, wherein the polypeptide comprises a Methyltransf_{—}12 signature sequence comprising amino acids 45 to 138 of SEQ ID NO:96. Most preferably, the transgenic plant of this embodiment comprises a polynucleotide encoding a precorrin-6b methylase having a sequence comprising amino acids 1 to 197 of SEQ ID NO:96.

### V. Decarboxylating Precorrin-6y Methylases

In another embodiment, the invention provides a transgenic plant transformed with an expression cassette comprising, in operative association, an isolated polynucleotide encoding a promoter and an isolated polynucleotide encoding a full-length decarboxylating precorrin-6y c5,15-methyltransferase polypeptide, wherein the transgenic plant demonstrates increased yield as compared to a wild type plant of the same variety which does not comprise the expression cassette. The expression cassette of this embodiment may optionally comprise an isolated polynucleotide encoding a mitochondrial transit peptide. As demonstrated in Example 2 below, transgenic Arabidopsis plants containing the Synechocystis sp. gene sll0099 (SEQ ID NO:97), with and without targeting to the mitochondria, demonstrate increased biomass as compared to control Arabidopsis plants. Gene sll0099 encodes a decarboxylating precorrin-6y methylase characterized, in part, by the presence of a TP_methylase signature sequence.

The transgenic plant of this embodiment may comprise any polynucleotide encoding a decarboxylating precorrin-6y methylase. Preferably, the transgenic plant of this embodiment comprises a polynucleotide encoding a full-length polypeptide having decarboxylating precorrin-6y methylase activity, wherein the polypeptide comprises a TP_methylase signature sequence comprising of amino acids 1 to 195 of SEQ ID NO:98. Most preferably, the transgenic plant of this embodiment comprises a polynucleotide encoding a decarboxylating precorrin-6y methylase having a sequence comprising amino acids 1 to 425 of SEQ ID NO:98.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO:19; SEQ ID NO:25; SEQ ID NO:27; SEQ ID NO:29; SEQ ID NO:31; SEQ ID NO:33; SEQ ID NO:37; SEQ ID NO:41; SEQ ID NO:43; SEQ ID NO:63; SEQ ID NO:49; SEQ ID NO:51; SEQ ID NO:53; SEQ ID NO:59; SEQ ID NO:63; SEQ ID NO:73, SEQ ID NO:75, SEQ ID NO:81, SEQ ID NO:83, SEQ ID NO:85, and SEQ ID NO:87. Also encompassed by the isolated polynucleotide of the invention is an isolated polynucleotide encoding a polypeptide having an amino acid sequence selected from the group consisting of SEQ ID NO:20; SEQ ID NO:26; SEQ ID NO:28; SEQ ID NO:30; SEQ ID NO:32; SEQ ID NO:36; SEQ ID NO:38; SEQ ID NO:42; SEQ ID NO:44; SEQ ID NO:64; SEQ ID NO:50; SEQ ID NO:52; SEQ ID NO:54; SEQ ID NO:60; SEQ ID NO:64; SEQ ID NO:74, SEQ ID NO:76, SEQ ID NO:82, SEQ ID NO:84, SEQ ID NO:86, and SEQ ID NO:88. A polynucleotide of the invention can be isolated using standard molecular biology techniques and the sequence information provided herein, for example, using an automated DNA synthesizer.

The isolated polynucleotides of the invention include homologs of the polynucleotides of Table 1. “Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide.

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI 9.0 (PC) software package (Invitrogen, 1600 Faraday Ave., Carlsbad, CA92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

The invention further provides a recombinant expression vector which comprises an expression cassette selected from the group consisting of a) an expression cassette comprising, in operative association, an isolated polynucleotide encoding a promoter capable of enhancing gene expression in leaves and an isolated polynucleotide encoding a full-length polypeptide having a sequence as set forth in SEQ ID NO:2; SEQ ID NO:4; SEQ ID NO:6; SEQ ID NO:8, or SEQ ID NO:22; b) an expression cassette comprising, in operative association, an isolated polynucleotide encoding a promoter capable of enhancing gene expression in leaves; an isolated polynucleotide encoding a plastid transit peptide; and an isolated polynucleotide encoding a full-length polypeptide having a sequence as set forth in SEQ ID NO:10, SEQ ID NO:12, or SEQ ID NO:14; c) an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length transcriptional regulator of fatty acid metabolism having a gntR-type HTH DNA-binding domain comprising amino acids 34 to 53 of SEQ ID NO:16; d) an expression cassette comprising, in operative association, an isolated polynucleotide encoding a promoter capable of enhancing gene expression in leaves; and an isolated polynucleotide encoding a full-length polypeptide having a G3E, P-loop domain comprising a Walker A motif having a sequence as set forth in SEQ ID NO:99 and a GTP-specificity motif having a sequence as set forth in SEQ ID NO:100; e) an expression cassette comprising in operative association, an isolated polynucleotide encoding a promoter capable of enhancing gene expression in leaves; an isolated polynucleotide encoding a mitochondrial transit peptide; and an isolated polynucleotide encoding a full-length peroxisomal-coenzyme A synthetase polypeptide comprising an AMP-binding domain selected from the group consisting of amino acids 194 to 205 of SEQ ID NO:24, amino acids 202 to 213 of SEQ ID NO:26, amino acids 214 to 225 of SEQ ID NO:28, amino acids 195 to 206 of SEQ ID NO:30, amino acids 175 to 186 of SEQ ID NO:32, amino acids 171 to 182 of SEQ ID NO:34, amino acids 189 to 200 of SEQ ID NO:36, amino acids 201 to 212 of SEQ ID NO:38; f) an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length histone H4 polypeptide having a G-A-K-R-H (SEQ ID NO:101) signature sequence domain selected from the group consisting of amino acids 3 to 92 of SEQ ID NO:40; amino acids 3 to 92 of SEQ ID NO:56; amino acids 3 to 92 of SEQ ID NO:42; and amino acids 3 to 92 of SEQ ID NO:44; g) an expression cassette comprising in operative association, an isolated polynucleotide encoding a promoter capable of enhancing gene expression in leaves or a constitutive promoter; an isolated polynucleotide encoding a chloroplast transit peptide; and polynucleotide encoding a full-length SYM1-type integral membrane protein; h) an expression cassette comprising in operative association, an isolated polynucleotide encoding a promoter capable of enhancing gene expression in leaves; an isolated polynucleotide encoding a mitochondrial transit peptide, and an isolated polynucleotide encoding a full-length vacuolar proton pump subunit H polypeptide; i) an expression cassette comprising in operative association, an isolated polynucleotide encoding a promoter capable of enhancing gene expression in leaves; an isolated polynucleotide encoding a mitochondrial transit peptide; and an isolated polynucleotide encoding a F-ATPase subunit alpha polypeptide comprising an ATP synthase domain selected from the group consisting of amino acids 356 to 365 of SEQ ID NO:62; amino acids 254 to 263 of SEQ ID NO:64; j) an expression cassette comprising, in operative association, an isolated polynucleotide encoding a promoter capable of enhancing gene expression in leaves; an isolated polynucleotide encoding a mitochondrial transit peptide; and an isolated polynucleotide encoding a full-length F-ATPase subunit beta polypeptide having a sequence as set forth in SEQ ID NO:66 k) an expression cassette comprising, in operative association, an isolated polynucleotide encoding a promoter; an isolated polynucleotide encoding a mitochondrial transit peptide; and an isolated polynucleotide encoding a full-length ABC transporter polypeptide having a sequence as set forth in SEQ ID NO:68; l) an expression cassette comprising in operative association, an isolated polynucleotide encoding a promoter; an isolated polynucleotide encoding a chloroplast transit peptide; and an isolated polynucleotide encoding a full-length PS-I subunit psaK polypeptide having a psaGK signature comprising amino acids 56 to 73 of SEQ ID NO:70; m) an expression cassette comprising in operative association, an isolated polynucleotide encoding a promoter; an isolated polynucleotide encoding a mitochondrial transit peptide; and an isolated polynucleotide encoding a full-length ferredoxin polypeptide comprising a Fer2 signature sequence selected from the group consisting of amino acids 11 to 87 of SEQ ID NO:72; amino acids 12 to 88 of SEQ ID NO:74; amino acids 63 to 139 of SEQ ID NO:76; n) an expression cassette comprising in operative association, an isolated polynucleotide encoding a promoter; an isolated polynucleotide encoding a chloroplast transit peptide; and an isolated polynucleotide encoding a full-length flavodoxin polypeptide having a Flavidoxin_{—}1 signature sequence comprising amino acids 6 to 160 of SEQ ID NO:78; o) an expression cassette comprising in operative association, an isolated polynucleotide encoding a promoter; an isolated polynucleotide encoding a chloroplast transit peptide; and an isolated polynucleotide encoding a full-length PS-I psaF polypeptide comprising a PSI_PsaF signature sequence selected from the group consisting of amino acids 3 to 158 of SEQ ID NO:80; amino acids 43 to 217 of SEQ ID NO:82; amino acids 46 to 220 of SEQ ID NO:84; amino acids 50 to 224 of SEQ ID NO:86; and amino acids 50 to 224 of SEQ ID NO:88; p) an expression cassette comprising in operative association, an isolated polynucleotide encoding a promoter; an isolated polynucleotide encoding a mitochondrial transit peptide; and an isolated polynucleotide encoding a full-length cytochrome c553 (petJ) polypeptide having aPSI_PsaF signature sequence comprising amino acids 38 to 116 of SEQ ID NO:90; q) an expression cassette comprising in operative association, an isolated polynucleotide encoding a promoter; an isolated polynucleotide encoding a mitochondrial transit peptide; and an isolated polynucleotide encoding a full-length PS-II W (PsbW) polypeptide having a Cytochrome C signature sequence comprising amino acids 5 to 120 of SEQ ID NO:92; r) an expression cassette comprising in operative association, an isolated polynucleotide encoding a promoter; an isolated polynucleotide encoding a chloroplast transit peptide; and an isolated polynucleotide encoding a full-length uroporphyrin-III c-methyltransferase (CobA) polypeptide having a sequence as set forth in SEQ ID NO:92; s) an expression cassette comprising in operative association, an isolated polynucleotide encoding a promoter and an isolated polynucleotide encoding a full-length precorrin-6b methylase polypeptide having a Methyltransf_{—}12 signature sequence comprising amino acids 45 to 138 of SEQ ID NO:96; and t) an expression cassette comprising in operative association, an isolated polynucleotide encoding a promoter and an isolated polynucleotide encoding a full-length decarboxylating precorrin-6y c5,15-methyltransferase having a TP_methylase signature sequence comprising amino acids 1 to 195 of SEQ ID NO:98.

In another embodiment, the recombinant expression vector of the invention comprises an isolated polynucleotide having a sequence selected from the group consisting of SEQ ID NO:19; SEQ ID NO:25; SEQ ID NO:27; SEQ ID NO:29; SEQ ID NO:31; SEQ ID NO:33; [SEQ ID NO:35?] SEQ ID NO:37; SEQ ID NO:41; SEQ ID NO:43; SEQ ID NO:63; SEQ ID NO:49; SEQ ID NO:51; SEQ ID NO:53; [SEQ ID NO:55?] SEQ ID NO:59; SEQ ID NO:63; SEQ ID NO:73, SEQ ID NO:75, SEQ ID NO:81, SEQ ID NO:83, SEQ ID NO:85, and SEQ ID NO:87. In addition, the recombinant expression vector of the invention comprises an isolated polynucleotide encoding a polypeptide having an amino acid sequence selected from the group consisting of SEQ ID NO:20; SEQ ID NO:26; SEQ ID NO:28; SEQ ID NO:30; SEQ ID NO:32; SEQ ID NO:36; SEQ ID NO:38; SEQ ID NO:42; SEQ ID NO:44; SEQ ID NO:64; SEQ ID NO:50; SEQ ID NO:52; SEQ ID NO:54; SEQ ID NO:60; SEQ ID NO:64; SEQ ID NO:74, SEQ ID NO:76, SEQ ID NO:82, SEQ ID NO:84, SEQ ID NO:86, and SEQ ID NO:88.

The recombinant expression vector of the invention includes one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

Such a combination of one or more regulatory sequences, selected on the basis of the host cells to be used for expression, in operative association with said polynucleotide is known in the art as the typical elements of an “expression cassette”. Such an expression cassette may further contain a chloroplast or mitochondrial transit sequence as defined below, linked to said polynucleotide. Expression cassettes are often described in the art as “constructs” and the two terms are used equivalently herein.

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (Baeumlein et al. (1991) Mol. Gen. Genet. 225, 459-67), promoters of light-inducible genes such as ribulose-1.5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105,357-67) and other leaf specific promoters such as those identified in Aleman, I. (2001) Isolation and characterization of leaf-specific promoters from alfalfa (Medicago sativa), Masters thesis, New Mexico State University, Los Cruces, N.M.

In other embodiments of the invention, a root or shoot specific promoter is employed. For example, the Super promoter provides high level expression in both root and shoots (Ni et al. (1995) Plant J. 7: 661-676). Other root specific promoters include, without limitation, the TobRB7 promoter (Yamamoto et al. (1991) Plant Cell 3, 371-382), the rolD promoter (Leach et al. (1991) Plant Science 79, 69-76); CaMV 35S Domain A (Benfey et al. (1989) Science 244, 174-181), and the like.

In other embodiments, a constitutive promoter is employed. Constitutive promoters are active under most conditions. Examples of constitutive promoters suitable for use in these embodiments include the parsley ubiquitin promoter described in WO 2003/102198 (SEQ ID NO:102) the CaMV 19S and 35S promoters, the sX CaMV 35S promoter, the Sep1 promoter, the rice actin promoter, the Arabidopsis actin promoter, the maize ubiquitin promoter, pEmu, the figwort mosaic virus 35S promoter, the Smas promoter, the super promoter (U.S. Pat. No. 5, 955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In accordance with the invention, a chloroplast transit sequence refers to a nucleotide sequence that encodes a chloroplast transit peptide. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); ferredoxin (Jansen et al. (1988) Curr. Genetics 13:517-522) (SEQ ID NO:111); nitrite reductase (Back et al (1988) MGG 212:20-26) and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

As defined herein, a mitochondrial transit sequence refers to a nucleotide sequence that encodes a mitochondrial presequence and directs the protein to mitochondria. Examples of mitochondrial presequences include groups consisting of ATPase subunits, ATP synthase subunits, Rieske-FeS protein, Hsp60, malate dehydrogenase, citrate synthase, aconitase, isocitrate dehydrogenase, pyruvate dehydrogenase, malic enzyme, glycine decarboxylase, serine hydroxymethyl transferase, isovaleryl-CoA dehydrogenase and superoxide dismutase. Such transit peptides are known in the art. See, for example, Von Heijne et al. (1991) Plant Mol. Biol. Rep. 9:104-126; Clark et al. (1989) J. Biol. Chem. 264:17544-17550; Romer et al. (1993) Biochem. Biophys. Res. Commun. 196:1414-1421; Faivre-Nitschke et al (2001) Eur J Biochem 268 1332-1339; Daschner et al. (1999) 39:1275-1282 (SEQ ID NO:109 and SEQ ID NO:107); and Shah et al. (1986) Science 233:478-481.

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example any of the techniques described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression cassette described above, (b) regenerating a transgenic plant from the transformed plant cell; and selecting higher-yielding plants from the regenerated plant sells. The plant cell may be, but is not limited to, a protoplast, gamete producing cell, and a cell that regenerates into a whole plant. As used herein, the term “transgenic” refers to any plant, plant cell, callus, plant tissue, or plant part, that contains the expression cassette described above. In accordance with the invention, the expression casette is stably integrated into a chromosome or stable extra-chromosomal element, so that it is passed on to successive generations.

The effect of the genetic modification on plant growth and/or yield and/or stress tolerance can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, lipid synthesis, evapotranspiration rates, general plant and/or crop yield, flowering, reproduction, seed setting, root growth, respiration rates, photosynthesis rates, metabolite composition, and the like.

The invention is further illustrated by the following examples, which are not to be construed in any way as imposing limitations upon the scope thereof.

### EXAMPLE 1

### Characterization of Genes

Genes B0821 (SEQ ID NO:1), B1187 (SEQ ID NO:15), B2173 (SEQ ID NO:17), B2668 (SEQ ID NO:3), B2670 (SEQ ID NO:21), B3362 (SEQ ID NO:5), B3555 (SEQ ID NO:7), SLL1911 (SEQ ID NO:9), SLR1062 (SEQ ID NO:11), YBR222C (SEQ ID NO:23), YDL193W (SEQ ID NO:13), YNL030W (SEQ ID NO:39), YLR251W (SEQ ID NO:45), YPR036W (SEQ ID NO:57), SLL1326 (SEQ ID NO:61), SLR1329 (SEQ ID NO:65), SLR0977 (SEQ ID NO:67), ssr0390 (SEQ ID NO:69), sll1382 (SEQ ID NO:71), sll0248 (SEQ ID NO:77), sll0819 (SEQ ID NO:79), sll1796 (SEQ ID NO:89), slr1739 (SEQ ID NO:91), sll0378 (SEQ ID NO:93), slr1368 (SEQ ID NO:95), and sll0099 (SEQ ID NO:97) were cloned using standard recombinant techniques. The functionality of each gene was predicted by comparing the predicted amino acid sequence of the gene with other genes of known functionality. Homolog cDNAs were isolated from proprietary libraries of the respective species using known methods. Sequences were processed and annotated using bioinformatics analyses. The degrees of amino acid identity and similarity of the isolated sequences to the respective closest known public sequences were used in the selection of homologous sequences as described below. Pairwise Comparison was used: gap penalty: 11; gap extension penalty: 1; score matrix: blosum62.

B2173 (SEQ ID NO:17) is a nucleotide-binding domain protein gene. The full-length predicted amino acid sequence of this gene was blasted against a proprietary database of predicted soybean amino acid sequences at an e value of e⁻¹⁰ (Altschul et al., supra). One homolog each from soybean and maize were identified. The amino acid relatedness of these sequences is indicated in the alignments shown in FIG. 1.

The full-length DNA sequence of YBR222C (SEQ ID NO:23) encodes a peroxisomal-coenzyme A synthetase from S. cerevisiae. The full-length predicted amino acid sequence of this gene was blasted against proprietary databases of canola, soybean, rice and maize cDNAs at an e value of e⁻¹⁰ (Altschul et al., supra). Three homologs from canola and four from soybean were identified. The amino acid relatedness of these sequences is indicated in the alignments shown in FIG. 2.

The full-length DNA sequence of YNL030W (SEQ ID NO:39) encodes a histone H4 from S. cerevisiae. The full-length predicted amino acid sequence of this gene was blasted against proprietary databases of rice and linseed cDNAs at an e value of e⁻¹⁰ (Altschul et al., supra). One homolog each from rice and linseed was identified. The amino acid relatedness of these sequences is indicated in the alignments shown in FIG. 3.

YLR251W (SEQ ID NO:45) is a SYM1-type integral membrane protein. The full-length predicted amino acid sequence of this gene was blasted against proprietary predicted amino acid sequence databases of canola, barley, soybean, linseed and rice at an e value of e⁻¹⁰ (Altschul et al., supra). One homolog from each library was identified. The amino acid relatedness of these sequences is indicated in the alignments shown in FIG. 4.

YPR036W (SEQ ID NO:57) is a vacuolar proton pump subunit H protein. The full-length predicted amino acid sequence of this gene was blasted against a proprietary predicted amino acid sequence database of canola at an e value of e⁻¹⁰ (Altschul et al., supra). One homolog from canola was identified. The amino acid relatedness of these sequences is indicated in the alignments shown in FIG. 5.

SLL1326 (SEQ ID NO:61) is an ATP synthase subunit alpha protein. The full-length predicted amino acid sequence of this gene was blasted against proprietary predicted amino acid sequence databases at an e value of e⁻¹⁰ (Altschul et al., supra). One homolog from the linseed library was identified. The amino acid relatedness of these sequences is indicated in the alignments shown in FIG. 6.

The sll1382 (SEQ ID NO:71) gene encodes ferredoxin in Synechocystis sp. The full-length amino acid sequence of sll1382 was blasted against a proprietary database of cDNAs at an e value of e⁻¹⁰ (Altschul et al., supra). One homolog from canola and one homolog from soybean were identified. The amino acid relatedness of these sequences is indicated in the alignments shown in FIG. 7.

The sll0819 (SEQ ID NO:79) gene encodes photosystem I reaction center subunit III in Synechocystis sp. The full-length amino acid sequence of sll0819 was blasted against a proprietary database of cDNAs at an e value of e⁻¹⁰ (Altschul et al., supra). Two homologs from canola and two homologs from soybean were identified. The amino acid relatedness of these sequences is indicated in the alignments shown in FIG. 8.

### EXAMPLE 2

### Overexpression of Selected Genes in Plants

The polynucleotides of Table 1 were ligated into an expression cassette using known methods. Three different promoters were used to control expression of the transgenes in Arabidopsis: the USP promoter from Vida faba (SEQ ID NO:104) was used for expression of genes from E. coli and cyanobacteria or SEQ ID NO:105 was used for expression of genes from S. cerevisiae); the super promoter (SEQ ID NO:103); and the parsley ubiquitin promoter (SEQ ID NO:102). For selective targeting of the polypeptides, the mitochondrial transit peptide from an A. thaliana gene encoding mitochondrial isovaleryl-CoA-dehydrogenase designated “Mito” in Tables 8, 9, 12, 13, 15-18, 20-25 and 27. SEQ ID NO:107 was used for expression of genes from E. coli and cyanobacteria or SEQ ID NO:109 was used for expression of genes from S. cerevisiae. In addition, for targeted expression, the chloroplast transit peptide of an Spinacia oleracea gene encoding ferredoxin nitrite reductase designated “ Chlor” in Tables 6, 14, 16, 17, 19-23 and 25 (SEQ ID NO:111) was used.

The Arabidopsis ecotype C24 was transformed with constructs containing the genes described in Example 1 using known methods. Seeds from T2 transformed plants were pooled on the basis of the promoter driving the expression, gene source species and type of targeting (chloroplastic, mitochondrial and none- the latter meaning no additional targeting signals were added). The seed pools were used in the primary screens for biomass under well watered and water limited growth conditions. Hits from pools in the primary screen were selected, molecular analysis performed and seed collected. The collected seeds were then used for analysis in secondary screens where a larger number of individuals for each transgenic event were analyzed. If plants from a construct were identified in the secondary screen as having increased biomass compared to the controls, it passed to the tertiary screen. In this screen, over 100 plants from all transgenic events for that construct were measured under well watered and drought growth conditions. The data from the transgenic plants were compared to wild type Arabidopsis plants or to plants grown from a pool of randomly selected transgenic Arabidopsis seeds using standard statistical procedures.

Plants that were grown under well watered conditions were watered to soil saturation twice a week. Images of the transgenic plants were taken at 17 and 21 days using a commercial imaging system. Alternatively, plants were grown under water limited growth conditions by watering to soil saturation infrequently which allowed the soil to dry between watering treatments. In these experiments, water was given on days 0, 8, and 19 after sowing. Images of the transgenic plants were taken at 20 and 27 days using a commercial imaging system.

Image analysis software was used to compare the images of the transgenic and control plants grown in the same experiment. The images were used to determine the relative size or biomass of the plants as pixels and the color of the plants as the ratio of dark green to total area. The latter ratio, termed the health index, was a measure of the relative amount of chlorophyll in the leaves and therefore the relative amount of leaf senescence or yellowing and was recorded at day 27 only. Variation exists among transgenic plants that contain the various genes, due to different sites of DNA insertion and other factors that impact the level or pattern of gene expression.

Tables 2 to 27 show the comparison of measurements of the Arabidopsis plants. Percent change indicates the measurement of the transgenic relative to the control plants as a percentage of the control non-transgenic plants; p value is the statistical significance of the difference between transgenic and control plants based on a T-test comparison of all independent events where NS indicates not significant at the 5% level of probabilty; No. of events indicates the total number of independent transgenic events tested in the experiment; positive events indicates the total number of independent transgenic events that were larger than the control in the experiment; negative events indicates the total number of independent transgenic events that were smaller than the control in the experiment. NS indicates not significant at the 5% level of probability.

### A. Untargeted Unknown Proteins

The protein designated B0821 (SEQ ID NO:2) was expressed in Arabidopsis using a construct wherein B0821 expression is controlled by the Super promoter and no exogenous targeting sequence is added to SEQ ID NO:2. Table 2 sets forth biomass and health index data obtained from the Arabidopsis plants transformed with these constructs and tested under water-limiting conditions.

TABLE 2

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B0821 | None | Biomass at | C24 | −0.50 | 0.8531 | 7 | 4 | 3 |
| | | day 20 | | | | | | |
| B0821 | None | Biomass at | C24 | 2.46 | 0.4884 | 7 | 5 | 2 |
| | | day 27 | | | | | | |
| B0821 | None | Health Index | C24 | −5.56 | 0.0115 | 7 | 1 | 6 |
| B0821 | None | Biomass at | Super | 9.11 | 0.0086 | 7 | 6 | 1 |
| | | day 20 | Pool | | | | | |
| B0821 | None | Biomass at | Super | 22.84 | 0.0000 | 7 | 5 | 2 |
| | | day 27 | Pool | | | | | |
| B0821 | None | Health Index | Super | −1.35 | 0.5720 | 7 | 3 | 4 |
| | | | Pool | | | | | |

Table 2 shows that Arabidopsis plants expressing B0821 (SEQ ID NO:2) that were grown under water limiting conditions were significantly larger than the control plants that did not express B0821 (SEQ ID NO:2) at day 27. Table 2 also shows that the majority of independent transgenic events were larger than the controls.

The B2668 gene (SEQ ID NO:4), which encodes a protein of unknown function, was expressed in Arabidopsis using a construct wherein transcription is controlled by the Super promoter. Table 3 sets forth biomass and health index data obtained from Arabidopsis plants transformed with these constructs and tested under water-limiting conditions.

TABLE 3

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B2668 | None | Biomass at | MTXC24 | −4.35 | 0.2162 | 7 | 3 | 4 |
| | | day 20 | | | | | | |
| B2668 | None | Biomass at | MTXC24 | 20.39 | 0.0000 | 6 | 6 | 0 |
| | | day 20 | | | | | | |
| B2668 | None | Biomass at | MTXC24 | −1.39 | 0.6577 | 7 | 3 | 4 |
| | | day 27 | | | | | | |
| B2668 | None | Biomass at | MTXC24 | 19.06 | 0.0000 | 6 | 6 | 0 |
| | | day 27 | | | | | | |
| B2668 | None | Health Index | MTXC24 | −3.17 | 0.1154 | 7 | 1 | 6 |
| B2668 | None | Health Index | MTXC24 | 0.49 | 0.8515 | 6 | 3 | 3 |
| B2668 | None | Biomass at | Super | 18.92 | 0.0000 | 7 | 7 | 0 |
| | | day 20 | Pool | | | | | |
| B2668 | None | Biomass at | Super | 9.96 | 0.0007 | 6 | 6 | 0 |
| | | day 20 | Pool | | | | | |
| B2668 | None | Biomass at | Super | 14.79 | 0.0001 | 7 | 7 | 0 |
| | | day 27 | Pool | | | | | |

Table 3 shows that Arabidopsis plants grown under water-limiting conditions were significantly larger than the control plants in two of three experiments. Table 3 also shows that the majority of independent transgenic events were larger than the controls.

The B3362 gene (SEQ ID NO:6), which encodes a protein of unknown function, was expressed in Arabidopsis using a construct wherein transcription is controlled by the Super promoter. Table 4 sets forth biomass and health index data obtained from Arabidopsis plants transformed with this construct and tested under water-limiting conditions.

TABLE 4

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B3362 | None | Biomass at | MTXC24 | 24.91 | 0.0000 | 7 | 7 | 0 |
| | | day 20 | | | | | | |
| B3362 | None | Biomass at | MTXC24 | 14.45 | 0.0015 | 7 | 5 | 2 |
| | | day 27 | | | | | | |
| B3362 | None | Health Index | MTXC24 | 11.97 | 0.0000 | 7 | 6 | 1 |
| B3362 | None | Biomass at | SuperPool | 35.78 | 0.0000 | 7 | 7 | 0 |
| | | day 20 | | | | | | |
| B3362 | None | Biomass at | SuperPool | 11.81 | 0.0069 | 7 | 5 | 2 |
| | | day 27 | | | | | | |
| B3362 | None | Health Index | SuperPool | 11.90 | 0.0000 | 7 | 6 | 1 |

Table 4 shows that Arabidopsis plants expression of B3362 (SEQ ID NO:6) were significantly larger than the control plants when the plants were grown under water-limiting conditions. Table 4 also shows that the majority of independent transgenic events were larger than the controls. In addition, this construct significantly increased the amount of green color of the plants when grown under water-limiting conditions.

The B3555 gene (SEQ ID NO:8), which encodes a protein of unknown function, was expressed in Arabidopsis using a construct wherein transcription is controlled by the Super promoter. Table 5 sets forth biomass and health index data obtained from Arabidopsis plants transformed with this construct and tested under water-limiting conditions.

TABLE 5

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B3555 | None | Biomass at | MTXC24 | −2.15 | 0.5969 | 6 | 2 | 4 |
| | | day 20 | | | | | | |
| B3555 | None | Biomass at | MTXC24 | 8.93 | 0.0388 | 6 | 4 | 2 |
| | | day 27 | | | | | | |
| B3555 | None | Health Index | MTXC24 | 0.16 | 0.9248 | 6 | 3 | 3 |
| B3555 | None | Biomass at | SuperPool | 5.91 | 0.2049 | 6 | 3 | 3 |
| | | day 20 | | | | | | |
| B3555 | None | Biomass at | SuperPool | 10.16 | 0.0273 | 6 | 5 | 1 |
| | | day 27 | | | | | | |
| B3555 | None | Health Index | SuperPool | 3.49 | 0.0450 | 6 | 4 | 2 |

Table 5 shows that Arabidopsis plants expressing B3555 (SEQ ID NO:8) were generally significantly larger than the control plants when the plants were grown under water-limiting conditions. Table 5 also shows that the majority of independent transgenic events were larger than the controls. In addition, this construct significantly increased the amount of green color of the plants when grown under water-limiting conditions when compared to the SuperPool controls.

### B. Plastid-Targeted Unknown Proteins

The SLL1911 gene (SEQ ID NO:10), which encodes a protein of unknown function, was expressed in Arabidopsis using two constructs wherein transcription is controlled by the PcUbi promoter. In one construct, a chloroplast targeting peptide was operatively linked to SEQ ID NO:10, whereas the other construct has no exogenous targeting peptide. Table 6 sets forth biomass and health index data obtained from Arabidopsis plants transformed with this construct and tested under water-limiting conditions.

TABLE 6

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| SLL1911 | None | Biomass at | MTXC24 | −38.77 | 0.0000 | 4 | 0 | 4 |
| | | day 20 | | | | | | |
| SLL1911 | None | Biomass at | MTXC24 | −19.00 | 0.0000 | 4 | 0 | 4 |
| | | day 27 | | | | | | |
| SLL1911 | None | Health Index | MTXC24 | −15.09 | 0.0000 | 4 | 0 | 4 |
| SLL1911 | None | Biomass at | SuperPool | −31.02 | 0.0000 | 4 | 0 | 4 |
| | | day 20 | | | | | | |
| SLL1911 | None | Biomass at | SuperPool | −13.85 | 0.0002 | 4 | 0 | 4 |
| | | day 27 | | | | | | |
| SLL1911 | None | Health Index | SuperPool | −12.79 | 0.0000 | 4 | 0 | 4 |
| SLL1911 | Chlor | Biomass at | MTXC24 | 21.96 | 0.0000 | 6 | 5 | 1 |
| | | day 20 | | | | | | |
| SLL1911 | Chlor | Biomass at | MTXC24 | 17.60 | 0.0014 | 6 | 5 | 1 |
| | | day 27 | | | | | | |
| SLL1911 | Chlor | Health Index | MTXC24 | 14.17 | 0.0006 | 6 | 4 | 2 |
| SLL1911 | Chlor | Biomass at | SuperPool | 11.83 | 0.0019 | 6 | 5 | 1 |
| | | day 20 | | | | | | |
| SLL1911 | Chlor | Biomass at | SuperPool | 15.71 | 0.0039 | 6 | 5 | 1 |
| | | day 27 | | | | | | |
| SLL1911 | Chlor | Health Index | SuperPool | 4.44 | 0.2497 | 6 | 4 | 2 |

Table 6 shows that Arabidopsis plants expressing SLL1911 (SEQ ID NO:10) were significantly larger than the control plants when SLL1911 was targeted to the chloroplast and the plants were grown under water-limiting conditions. Table 6 also shows that the majority of independent transgenic events were larger than the controls when SLL1911 was targeted to the chloroplast. In addition, the construct wherein an exogenous chloroplast targeting peptide was operatively linked to SLL1911 significantly increased the amount of green color of the plants when grown under water-limiting conditions. These data indicate that the plants produced more chlorophyll or had less chlorophyll degradation during stress than the control plants when SLL1911 was operatively linked to a chloroplast targeting peptide. In contrast, when plants expressed a version of SLL1911 which lacked an exogenous chloroplast-targeting peptide, the resulting transgenic plants were significantly smaller and had significantly less green color when compared to control plants grown under the same water-limiting conditions. Together, these observations suggest that the subcellular localization of SLL1911 is essential to increase the size and amount of green color in transgenic plants expressing the SLL1911 gene.

The SLR1062 gene (SEQ ID NO:12), which encodes a protein of unknown function, was expressed in Arabidopsis using a construct wherein transcription is controlled by the PcUbi promoter. Table 7 sets forth biomass and health index data obtained from Arabidopsis plants transformed with this construct and tested under water-limiting conditions.

TABLE 7

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| SLR1062 | None | Biomass at | MTXC24 | −1.10 | 0.8087 | 6 | 4 | 2 |
| | | day 20 | | | | | | |
| SLR1062 | None | Biomass at | MTXC24 | 50.34 | 0.0000 | 5 | 5 | 0 |
| | | day 20 | | | | | | |
| SLR1062 | None | Biomass at | MTXC24 | 16.66 | 0.0009 | 6 | 5 | 1 |
| | | day 27 | | | | | | |
| SLR1062 | None | Biomass at | MTXC24 | 32.27 | 0.0000 | 5 | 4 | 1 |
| | | day 27 | | | | | | |
| SLR1062 | None | Health Index | MTXC24 | −15.63 | 0.0000 | 6 | | 6 |
| SLR1062 | None | Health Index | MTXC24 | 20.67 | 0.0000 | 5 | 4 | 1 |
| SLR1062 | None | Biomass at | SuperPool | 8.74 | 0.0716 | 5 | 4 | 1 |
| | | day 20 | | | | | | |
| SLR1062 | None | Biomass at | SuperPool | 7.63 | 0.0340 | 5 | 4 | 1 |
| | | day 27 | | | | | | |
| SLR1062 | None | Health Index | SuperPool | 8.62 | 0.0524 | 5 | 3 | 2 |

Table 7 shows that Arabidopsis plants expressing SLR1062 (SEQ ID NO:12) were generally significantly larger than the control plants when the plants were grown under water-limiting conditions. Table 7 also shows that the majority of independent transgenic events were larger than the controls. In addition, this construct significantly increased the amount of green color of the plants when grown under water-limiting conditions in two out of three observations. These data indicate that the plants produced more chlorophyll or had less chlorophyll degradation during stress than the control plants.

### C. Undecaprenyl Pyrophosphate Synthetase

The YDL193W gene (SEQ ID NO:14), which encodes a putative Undecaprenyl Pyrophosphate Synthetase protein, was expressed in Arabidopsis using a construct wherein transcription is controlled by the USP promoter and the polypeptide translated from the resulting transcript is operatively linked to a mitochondrial targeting peptide. Table 8 sets forth biomass and health index data obtained from Arabidopsis plants transformed with this construct and tested under water-limiting conditions.

TABLE 8

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| YDL193W | Mito | Biomass at | MTXC24 | 12.18 | 0.0014 | 7 | 6 | 1 |
| | | day 20 | | | | | | |
| YDL193W | Mito | Biomass at | MTXC24 | 9.45 | 0.0017 | 7 | 5 | 2 |
| | | day 27 | | | | | | |
| YDL193W | Mito | Health Index | MTXC24 | 3.05 | 0.3250 | 7 | 5 | 2 |
| YDL193W | Mito | Biomass at | SuperPool | 19.13 | 0.0000 | 7 | 6 | 1 |
| | | day 20 | | | | | | |
| YDL193W | Mito | Biomass at | SuperPool | 13.66 | 0.0000 | 7 | 6 | 1 |
| | | day 27 | | | | | | |
| YDL193W | Mito | Health Index | SuperPool | 10.90 | 0.0024 | 7 | 6 | 1 |

Table 8 shows that Arabidopsis plants expressing YDL193W (SEQ ID NO:14) were significantly larger than the control plants when the plants were grown under water-limiting conditions. Table 8 also shows that the majority of independent transgenic events were larger than the controls. In addition, this construct significantly increased the amount of green color of the plants when grown under water-limiting conditions. The greater amount of green color indicates that the plants produced more chlorophyll or had less chlorophyll degradation during stress than the control plants.

### D. Putative Transcriptional Regulator of Fatty Acid Metabolism

The putative transcriptional regulator of fatty acid metabolism designated B1187 (SEQ ID NO:16) was expressed in Arabidopsis using a construct wherein transcriptional regulator of fatty acid metabolism expression is controlled by the USP promoter and the transcriptional regulator of fatty acid metabolism is targeted to the mitochondria. Table 9 sets forth biomass and health index data obtained from the Arabidopsis plants transformed with these constructs and tested under well-watered conditions.

TABLE 9

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B1187 | Mito | Biomass at | MTXC24 | 29.94 | 0.0000 | 6 | 5 | 1 |
| | | day 20 | | | | | | |
| B1187 | Mito | Biomass at | MTXC24 | 13.57 | 0.0009 | 6 | 4 | 2 |
| | | day 27 | | | | | | |
| B1187 | Mito | Health Index | MTXC24 | 0.53 | 0.8751 | 6 | 4 | 2 |
| B1187 | Mito | Biomass at | Super | 26.50 | 0.0000 | 6 | 5 | 1 |
| | | day 20 | Pool | | | | | |
| B1187 | Mito | Biomass at | Super | 11.60 | 0.0061 | 6 | 4 | 2 |
| | | day 27 | Pool | | | | | |
| B1187 | Mito | Health Index | Super | 8.21 | 0.0233 | 6 | 6 | 0 |
| | | | Pool | | | | | |

Table 9 shows that Arabidopsis plants that were grown under well watered conditions were significantly larger than the control plants that did not express B1187 (SEQ ID NO:16). Table 9 also shows that all independent transgenic events were larger than the controls in the well watered environment.

### E. G3E-Family, P-Loop Domain, Nucleotide-Binding Protein

The B2173 gene (SEQ ID NO:18), which encodes a G3E-family, P-loop domain, nucleotide binding protein, was expressed in Arabidopsis using construct wherein transcription is controlled by the Super promoter. Table 10 sets forth biomass and health index data obtained from Arabidopsis plants transformed with these constructs and tested under water-limiting conditions.

TABLE 10

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B2173 | None | Biomass at | MTXC24 | −4.18 | 0.1622 | 6 | 2 | 4 |
| | | day 20 | | | | | | |
| B2173 | None | Biomass at | MTXC24 | −1.13 | 0.7435 | 6 | 2 | 4 |
| | | day 27 | | | | | | |
| B2173 | None | Health Index | MTXC24 | −4.54 | 0.0530 | 6 | 2 | 4 |
| B2173 | None | Biomass at | Super | 5.07 | 0.1725 | 6 | 4 | 2 |
| | | day 20 | Pool | | | | | |
| B2173 | None | Biomass at | Super | 18.54 | 0.0000 | 6 | 5 | 1 |
| | | day 27 | Pool | | | | | |
| B2173 | None | Health Index | Super | −0.29 | 0.9087 | 6 | 3 | 3 |
| | | | Pool | | | | | |

Table 10 shows that Arabidopsis plants with expressing B2173 (SEQ ID NO:18) were significantly larger than the SuperPool control plants. Table 10 also shows that the majority of independent transgenic events were larger than the SuperPool controls.

### F. Putative Membrane Protein

The B2670 gene (SEQ ID NO:22), which encodes a putative membrane protein, was expressed in Arabidopsis using a construct wherein transcription is controlled by the Super promoter. Table 11 sets forth biomass and health index data obtained from Arabidopsis plants transformed with the first two constructs and tested under water-limiting conditions.

TABLE 11

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| B2670 | None | Biomass at | MTXC24 | 18.87 | 0.0000 | 7 | 5 | 2 |
| | | day 20 | | | | | | |
| B2670 | None | Biomass at | MTXC24 | 15.41 | 0.0005 | 7 | 6 | 1 |
| | | day 27 | | | | | | |
| B2670 | None | Health Index | MTXC24 | 15.51 | 0.0000 | 7 | 7 | 0 |
| B2670 | None | Biomass at | Super | 29.22 | 0.0000 | 7 | 6 | 1 |
| | | day 20 | Pool | | | | | |
| B2670 | None | Biomass at | Super | 12.74 | 0.0027 | 7 | 5 | 2 |
| | | day 27 | Pool | | | | | |
| B2670 | None | Health Index | Super | 15.44 | 0.0000 | 7 | 7 | 0 |
| | | | Pool | | | | | |

Table 11 shows that Arabidopsis plants expressing B2670 (SEQ ID NO:22) were significantly larger than the control plants when grown under water-limiting conditions. In addition, these transgenic plants were darker green in color than the controls. These data indicate that the plants produced more chlorophyll or had less chlorophyll degradation during stress than the control plants. Table 11 also shows that the majority of independent transgenic events were larger than the controls.

### G. Peroxisomal Coenzyme A Synthetase

The YBR222C gene (SEQ ID NO:24), which encodes a peroxisomal-coenzyme A synthetase, was expressed in Arabidopsis using a construct wherein transcription is controlled by the USP promoter and the polypeptide translated from the resulting transcript is operatively linked to a mitochondrial targeting peptide. Table 12 sets forth biomass and health index data obtained from Arabidopsis plants transformed with this construct and tested under water-limiting conditions.

TABLE 12

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| YBR222C | Mito | Biomass at | MTXC24 | 10.55 | 0.0062 | 7 | 6 | 1 |
| | | day 20 | | | | | | |
| YBR222C | Mito | Biomass at | MTXC24 | 8.43 | 0.0134 | 7 | 4 | 3 |
| | | day 27 | | | | | | |
| YBR222C | Mito | Health Index | MTXC24 | 5.27 | 0.1015 | 7 | 5 | 2 |
| YBR222C | Mito | Biomass at | SuperPool | 34.10 | 0.0000 | 7 | 7 | 0 |
| | | day 20 | | | | | | |
| YBR222C | Mito | Biomass at | SuperPool | 13.28 | 0.0001 | 7 | 5 | 2 |
| | | day 27 | | | | | | |
| YBR222C | Mito | Health Index | SuperPool | 16.39 | 0.0000 | 7 | 7 | 0 |

Table 12 shows that Arabidopsis plants expressing YBR222C (SEQ ID NO:24) were significantly larger than the control plants when the plants were grown under water-limiting conditions. Table 12 also shows that the majority of independent transgenic events were larger than the controls. In addition, this construct significantly increased the amount of green color of the plants when grown under water-limiting conditions. The greater amount of green color indicates that the plants produced more chlorophyll or had less chlorophyll degradation during stress than the control plants.

### H. Histone H4

The YNL030W gene (SEQ ID NO:40), which encodes a histone H4, was expressed in Arabidopsis using a construct wherein transcription is controlled by the USP promoter and the polypeptide translated from the resulting transcript is operatively linked to a mitochondrial targeting peptide. Table 13 sets forth biomass and health index data obtained from Arabidopsis plants transformed with this construct and tested under well-watered conditions.

TABLE 13

| | | | | | | | No of | No. of |
|---|---|---|---|---|---|---|---|---|
| | | | Control | % | p- | No. of | Positive | Negative |
| Gene | Targeting | Measurement | Name | Change | Value | Events | Events | Events |
| YNL030W | Mito | Health Index | MTXC24 | 7.82 | 0.0521 | 6 | 4 | 2 |
| YNL030W | Mito | Health Index | MTXC24 | 10.28 | 0.0023 | 6 | 5 | 1 |
| YNL030W | Mito | Biomass at day 17 | MTXC24 | −6.06 | 0.0303 | 6 | 0 | 6 |
| YNL030W | Mito | Biomass at day 17 | MTXC24 | 29.50 | 0.0000 | 6 | 6 | 0 |
| YNL030W | Mito | Biomass at day 21 | MTXC24 | −6.73 | 0.0089 | 6 | 1 | 5 |
| YNL030W | Mito | Biomass at day 21 | MTXC24 | 20.78 | 0.0000 | 6 | 5 | 1 |
| YNL030W | Mito | Health Index | SuperPool | 4.76 | 0.2704 | 6 | 4 | 2 |
| YNL030W | Mito | Health Index | SuperPool | 0.50 | 0.8830 | 6 | 2 | 4 |
| YNL030W | Mito | Biomass at day 17 | SuperPool | 7.30 | 0.0281 | 6 | 5 | 1 |
| YNL030W | Mito | Biomass at day 17 | SuperPool | 13.14 | 0.0000 | 6 | 5 | 1 |
| YNL030W | Mito | Biomass at day 21 | SuperPool | 4.15 | 0.1583 | 6 | 5 | 1 |
| YNL030W | Mito | Biomass at day 21 | SuperPool | 9.31 | 0.0017 | 6 | 5 | 1 |

Table 13 shows that Arabidopsis plants expressing YNL030W (SEQ ID NO:40) were generally, significantly larger than the control plants when the plants were well watered. Table 13 also shows that the majority of independent transgenic events were larger than the controls. In addition, this construct significantly increased the amount of green color of the plants when grown under well-watered conditions and compared to the MTXC24 control. The greater amount of green color indicates that the plants produced more chlorophyll or had less chlorophyll degradation during stress than the control plants.

### I. Integral Membrane Protein SYM1

The integral membrane protein designated YLR251W (SEQ ID NO:45) was expressed in Arabidopsis using a construct wherein SYM1-type integral membrane protein expression is controlled by the USP, Super or PCUbi promoter and the integral membrane protein is targeted to chloroplasts. Table 14 sets forth biomass and health index data obtained from the Arabidopsis plants transformed with these constructs and tested under water-limiting (CD) and well-watered (WW) conditions.

TABLE 14

| | | | | | | | | No of | No. of |
|---|---|---|---|---|---|---|---|---|---|
| Assay | | | | | % | p- | No. of | Positive | Negative |
| Type | Gene | Promoter | Targeting | Measurement | Change | Value | Events | Events | Events |
| CD | YLR251W | PCUbi | Chlor | Biomass Day 20 | 35.6 | 0.000 | 6 | 6 | 0 |
| CD | YLR251W | PCUbi | Chlor | Biomass Day 27 | 26.3 | 0.000 | 6 | 6 | 0 |
| CD | YLR251W | PCUbi | Chlor | Health Index | 8.3 | 0.037 | 6 | 3 | 3 |
| CD | YLR251W | Super | Chlor | Biomass Day 20 | −20.4 | 0.000 | 6 | 1 | 5 |
| CD | YLR251W | Super | Chlor | Biomass Day 27 | −20.4 | 0.000 | 6 | 1 | 5 |
| CD | YLR251W | Super | Chlor | Health Index | −15.4 | 0.000 | 6 | 1 | 5 |
| CD | YLR251W | USP | Chlor | Biomass Day 20 | 12.5 | 0.003 | 7 | 5 | 2 |
| CD | YLR251W | USP | Chlor | Biomass Day 27 | 2.2 | NS | 7 | 4 | 3 |
| CD | YLR251W | USP | Chlor | Health Index | 10.5 | 0.007 | 7 | 5 | 2 |
| WW | YLR251W | PCUbi | Chlor | Biomass Day 17 | 32.7 | 0.000 | 6 | 6 | 0 |
| WW | YLR251W | PCUbi | Chlor | Biomass Day 21 | 27.4 | 0.000 | 6 | 6 | 0 |
| WW | YLR251W | PCUbi | Chlor | Health Index | 0.5 | NS | 6 | 4 | 2 |
| WW | YLR251W | Super | Chlor | Biomass Day 17 | −26.2 | 0.000 | 6 | 0 | 6 |
| WW | YLR251W | Super | Chlor | Biomass Day 21 | −17.4 | 0.000 | 6 | 0 | 6 |

Table 14 shows that transgenic plants expressing the YLR251W (SEQ ID NO:62) gene under the control of promoter PCUbi (SEQ ID NO:102) or USP (SEQ ID NO:104)with targeting to the plastid were significantly larger under either well-water or drought conditions than the control plants that did not express the YLR251W (SEQ ID NO:45) gene. In these experiments, all or the majority of the independent transgenic events with these two promoters were larger than the controls in the cycling drought environment. As evidenced by the observation that the transgenic plants were larger than the control under cycling drought conditions, the presence of the SYM1 protein in the plastid, when expressed using the USP or PCUbi promoters, resulted in improved transport efficiency and reduced detrimental effects due to the loss of water.

Table 14 shows that transgenic plants expressing the YLR251W (SEQ ID NO:45) gene under control of the Super promoter with targeting to the plastid were significantly smaller under either well-water or drought conditions than the control plants that did not express the YLR251W (SEQ ID NO:45) gene. These results indicated that the expression of YLR251W (SEQ ID NO:45) provided by the PCUbi and USP are important for the function of YLR251W (SEQ ID NO:45).

### J. Vacuolar Proton Pump Subunit H

The vacuolar proton pump subunit H protein designated YPR036W (SEQ ID NO:58) was expressed in Arabidopsis using a construct wherein vacuolar proton pump subunit H protein expression is controlled by the USP promoter and the vacuolar proton pump subunit H protein protein is targeted to mitochondria. Table 15 sets forth biomass and health index data obtained from Arabidopsis plants transformed with these constructs and tested under well-watered conditions.

TABLE 15

| | | | | | | | No of | No. of |
|---|---|---|---|---|---|---|---|---|
| Assay | | | | % | p- | No. of | Positive | Negative |
| Type | Gene | Targeting | Measurement | Change | Value | Events | Events | Events |
| CD | YPR036W | Mito | Biomass Day 20 | 21.3 | 0.000 | 7 | 6 | 1 |
| CD | YPR036W | Mito | Biomass Day 27 | 17.2 | 0.000 | 7 | 6 | 1 |
| CD | YPR036W | Mito | Health Index | 14.3 | 0.000 | 7 | 7 | 0 |
| WW | YPR036W | Mito | Biomass Day 17 | −12.5 | 0.000 | 7 | 3 | 4 |
| WW | YPR036W | Mito | Biomass Day 21 | −6.9 | 0.002 | 7 | 3 | 4 |
| WW | YPR036W | Mito | Health Index | 6.5 | NS | 7 | 6 | 1 |

Table 15 shows that transgenic plants expressing the YPR036W (SEQ ID NO:58) gene under control of the USP promoter with targeting to the mitochondria were significantly larger and healthier under drought conditions than the control plants that did not express the YPR036W (SEQ ID NO:58) gene. In these experiments, the majority of the independent transgenic events with mitochondria targeting were larger and healthier than the controls in the cycling drought environment. As evidenced by the observation that the transgenic plants were larger and healthier than the control under cycling drought conditions, the presence of the V-type ATPase subunit H protein in the mitochondria resulted in improved transport efficiency and reduced detrimental effects due to the loss of water.

K. F-ATPase subunit alpha

F-ATPase subunit alpha gene SLL1326 (SEQ ID NO:62) was expressed in Arabidopsis under control of the PCUbi promoter and targeted to the plastid and mitochondria or plastid. Table 16 sets forth biomass and health index data obtained from the Arabidopsis plants transformed with these constructs and tested under cycling drought conditions.

TABLE 16

| | | | | | | | No of | No. of |
|---|---|---|---|---|---|---|---|---|
| Assay | | | | % | p- | No. of | Positive | Negative |
| Type | Gene | Targeting | Measurement | Change | Value | Events | Events | Events |
| CD | sll1326 | Mito | Biomass Day 20 | 15.1 | 0.000 | 6 | 4 | 2 |
| CD | sll1326 | Mito | Biomass Day 27 | 15.4 | 0.000 | 6 | 4 | 2 |
| CD | sll1326 | Mito | Health Index | −4.9 | NS | 6 | 2 | 4 |
| CD | sll1326 | Chlor | Biomass Day 20 | −15.1 | 0.000 | 4 | 1 | 3 |
| CD | sll1326 | Chlor | Biomass Day 27 | −14.4 | 0.001 | 4 | 0 | 4 |
| CD | sll1326 | Chlor | Health Index | −6.0 | NS | 4 | 1 | 3 |

Table 16 shows that transgenic plants expressing the SLL1326 gene under control of the PCUbi promoter with targeting to the mitochondria were significantly larger under drought conditions than the control plants that did not express the SLL1326 gene. In these experiments, the majority of the independent transgenic events with mitochondrial targeting were larger than the controls in the cycling drought environment. As evidenced by the observation that the transgenic plants were larger than the control under cycling drought conditions, the presence of the F-ATPase subunit alpha protein in the mitochondria resulted in improved transport efficiency and reduced detrimental effects due to the loss of water.

Table 16 shows that transgenic plants expressing the SLL1326 gene under control of the PCUbi promoter with targeting to the plastid were significantly smaller and less healthy under drought conditions than the control plants that did not express the SLL1326 gene. Table 16 sets forth biomass and health index data obtained from Arabidopsis plants transformed with these constructs and tested under water-limiting conditions.

### L. F-ATPase Subunit Beta

F-ATPase subunit beta gene SLR1329 (SEQ ID NO:66) was expressed in Arabidopsis under control of the PCUbi promoter and targeted to the plastid or mitochondria. Table 17 sets forth biomass and health index data obtained from the Arabidopsis plants transformed with these constructs and tested under cycling drought or well-watered conditions.

TABLE 17

| | | | | | | | No of | No. of |
|---|---|---|---|---|---|---|---|---|
| Assay | | | | % | p- | No. of | Positive | Negative |
| Type | Gene | Targeting | Measurement | Change | Value | Events | Events | Events |
| CD | slr1329 | Mito | Biomass Day 20 | 12.8 | 0.000 | 6 | 5 | 1 |
| CD | slr1329 | Mito | Biomass Day 27 | 7.8 | 0.026 | 6 | 4 | 2 |
| CD | slr1329 | Mito | Health Index | 8.1 | 0.010 | 6 | 5 | 1 |
| CD | slr1329 | Chlor | Biomass Day 20 | −34.8 | 0.000 | 6 | 0 | 6 |
| CD | slr1329 | Chlor | Biomass Day 27 | −17.5 | 0.000 | 6 | 0 | 6 |
| CD | slr1329 | Chlor | Health Index | −15.9 | 0.000 | 6 | 1 | 5 |
| WW | slr1329 | Chlor | Biomass Day 17 | −13.7 | 0.000 | 5 | 1 | 4 |
| WW | slr1329 | Chlor | Biomass Day 21 | −9.9 | 0.000 | 5 | 0 | 5 |
| WW | slr1329 | Chlor | Health Index | 0.2 | NS | 5 | 2 | 3 |

Table 17 shows that transgenic plants expressing the SLR1329 (SEQ ID NO:66) gene under control of the PCUbi promoter with targeting to the mitochondria were significantly larger and healthier under drought conditions than the control plants that did not express the SLR1329 (SEQ ID NO:66) gene. In these experiments, the majority of the independent transgenic events with mitochondria targeting were larger than the controls in the cycling drought environment. As evidenced by the observation that the transgenic plants were larger than the control under cycling drought conditions, the presence of the F-ATPase subunit beta protein in the mitochondria resulted in improved transport efficiency and reduced detrimental effects due to the loss of water.

Table 17 shows that transgenic plants expressing the SLR1329 (SEQ ID NO:66) gene under control of the PCUbi promoter with targeting to the plastid were significantly smaller under drought and well-water conditions, significantly less healthy under drought conditions than the control plants that did not express the SLR1329 (SEQ ID NO:66) gene.

### M. ABC Transporter

ABC transporter gene SLR0977 (SEQ ID NO:68) was expressed in Arabidopsis under control of the PCUbi promoter and targeted to the mitochondria. Table 18 sets forth biomass and health index data obtained from the Arabidopsis plants transformed with this construct and tested under cycling drought and well-watered conditions.

TABLE 18

| | | | | | | | No of | No. of |
|---|---|---|---|---|---|---|---|---|
| Assay | | | | % | p- | No. of | Positive | Negative |
| Type | Gene | Targeting | Measurement | Change | Value | Events | Events | Events |
| CD | slr0977 | Mito | Biomass Day 20 | 14.3 | 0.000 | 6 | 6 | 0 |
| CD | slr0977 | Mito | Biomass Day 27 | 12.1 | 0.000 | 6 | 5 | 1 |
| CD | slr0977 | Mito | Health Index | 4.5 | NS | 6 | 5 | 1 |
| WW | slr0977 | Mito | Biomass Day 17 | −0.2 | NS | 6 | 3 | 3 |
| WW | slr0977 | Mito | Biomass Day 21 | −2.6 | NS | 6 | 2 | 4 |
| WW | slr0977 | Mito | Health Index | 9.0 | 0.010 | 6 | 5 | 1 |

Table 18 shows that transgenic plants expressing the SLR0977 gene under control of the PCUbi promoter with targeting to the mitochondria were significantly larger under drought conditions than the control plants that did not express the SLR0977 gene. In these experiments, all or the majority of the independent transgenic events with mitochondria targeting were larger than the controls in the cycling drought environment. As evidenced by the observation that the transgenic plants were larger than the control under cycling drought conditions, the presence of the ABC transporter protein in the mitochondria resulted in improved transport efficiency and reduced detrimental effects due to the loss of water.

### N. PsaK

The PsaK gene SSR0390 (SEQ ID NO:69) was expressed in Arabidopsis under control of the PcUbi promoter and targeted to the plastid. Table 19 sets forth biomass and health index data obtained from the Arabidopsis plants transformed with these constructs and tested under cycling drought conditions.

TABLE 19

| Assay | | | | Percent | | Valid | Positive | Negative |
|---|---|---|---|---|---|---|---|---|
| Type | Gene | Target | Trait | Change | pValue | Events | Events | Events |
| CD | ssr0390 | Chlor | Day 17 | 14.0 | 0.00 | 6 | 4 | 2 |
| CD | ssr0390 | Chlor | Day 21 | 6.8 | 0.01 | 6 | 4 | 2 |
| CD | ssr0390 | Chlor | Health Index | 4.4 | NS | 6 | 3 | 3 |

Table 19 shows that transgenic plants expressing the ssr0390 gene with targeting to the plastid were significantly larger under well-watered conditions than the control plants that did not express the ssr0390 gene. In these experiments, the majority of the independent transgenic events with plastid targeting were larger than the controls in the cycling drought environment. As evidenced by the observation that the transgenic plants were larger than the control under cycling drought conditions, the presence of the PsaK protein in the plastid resulted in improved photosynthetic efficiency and reduced detrimental effects due to the loss of water.

### O. Ferredoxin (PetF)

The ferredoxin (PetF) gene sll1382 (SEQ ID NO:71) was expressed in Arabidopsis using two different constructs, one under control of the PcUbi promoter and targeted to mitochondria, and the second with the same promoter targeted to the plastid. Table 20 sets forth biomass and health index data obtained from the Arabidopsis plants transformed with these constructs and tested under cycling drought and well watered conditions.

TABLE 20

| Assay | | | | Percent | | Valid | Positive | Negative |
|---|---|---|---|---|---|---|---|---|
| Type | Gene | Target | Trait | Change | pValue | Events | Events | Events |
| WW | sll1382 | Mito | Day 17 | 32.5 | 0.00 | 6 | 6 | 0 |
| WW | sll1382 | Mito | Day 21 | 19.2 | 0.00 | 6 | 6 | 0 |
| WW | sll1382 | Mito | Health Index | 0.2 | NS | 6 | 2 | 4 |
| WW | sll1382 | Chlor | Day 17 | 0.6 | NS | 6 | 3 | 3 |
| WW | sll1382 | Chlor | Day 20 | 1.0 | NS | 6 | 3 | 3 |
| WW | sll1382 | Chlor | Health Index | −0.7 | NS | 6 | 3 | 3 |
| CD | sll1382 | Mito | Day 20 | −0.3 | NS | 7 | 4 | 3 |
| CD | sll1382 | Mito | Day 27 | −11.3 | 0.00 | 7 | 1 | 6 |
| CD | sll1382 | Mito | Health Index | 4.0 | NS | 7 | 5 | 2 |
| CD | sll1382 | Chlor | Day 20 | −31.7 | 0.00 | 6 | 0 | 6 |
| CD | sll1382 | Chlor | Day 27 | −13.8 | 0.00 | 6 | 0 | 6 |
| CD | sll1382 | Chlor | Health Index | −8.1 | 0.00 | 6 | 0 | 6 |

Table 20 shows that transgenic plants expressing the sll1382 gene with targeting to the mitochondria were significantly larger under well-watered conditions than the control plants that did not express the sll1382 gene. Under water-limited conditions, the transgenic plants were significantly smaller than the controls when measured at day 27, and not significantly different at other measured timepoints or in health index.

Table 20 shows that transgenic plants expressing the sll1382 gene with targeting to the plastid were significantly smaller under water-limited conditions than the control plants that did not express the sll1382 gene. Additionally, these transgenic plants had lower health index scores relative to the control in water-limited conditions. In well-watered conditions, transgenic plants expressing the sll1382 gene gene with targeting to the plastid were not significantly different from the controls in biomass or health index. In these experiments, the majority of the independent transgenic events with mitochondrial targeting were larger than the controls in the either water environment.

These observations are consistent with previous reports indicating that ferredoxin did not improve plant growth when targeted to plastids in transgenic plants. As evidenced by the observation that the transgenic plants were larger than the control plants when the ferredoxin protein was targeted to the mitochondria, the presence of the ferrredoxin protein in the mitochondria resulted in improved electron transport efficiency.

### P. Flavodoxin

The flavodoxin gene sll0248 (SEQ ID NO:77) was expressed in Arabidopsis using two different constructs under control of the PcUbi promoter and targeted to mitochondria, or to the plastid. Table 21 sets forth biomass and health index data obtained from the Arabidopsis plants transformed with these constructs and tested under cycling drought and well watered conditions.

TABLE 21

| Assay | | | | Percent | | Valid | Positive | Negative |
|---|---|---|---|---|---|---|---|---|
| Type | Gene | Target | Trait | Change | pValue | Events | Events | Events |
| WW | sll0248 | Mito | Day 17 | −7.9 | 0.01 | 7 | 2 | 5 |
| WW | sll0248 | Mito | Day 21 | −3.9 | NS | 7 | 2 | 5 |
| WW | sll0248 | Mito | Health Index | −1.7 | NS | 7 | 2 | 5 |
| WW | sll0248 | Chlor | Day 17 | 11.1 | 0.00 | 6 | 5 | 1 |
| WW | sll0248 | Chlor | Day 21 | 10.0 | 0.00 | 6 | 5 | 1 |
| WW | sll0248 | Chlor | Health Index | −3.9 | NS | 6 | 1 | 5 |

Table 21 shows that transgenic plants expressing the sll0248 gene with targeting to the plastid were significantly larger under well-watered conditions than the control plants that did not express the sll0248 gene. Transgenic plants expressing the sll0248 gene with subcellular targeting to the mitochondria were significantly smaller under well-watered conditions at 17 days than the control plants that did not express the sll0248 gene, but not significantly different at 21 days from the control plants that did not express the sll0248 gene under the same conditions. Health index of the transgenic plants expressing either construct was not significantly different from the controls. In these experiments, the majority of the independent transgenic events with plastid targeting were larger than the controls in the either water environment and those with mitochondrial targeting were smaller than the controls in the well-watered environment.

As evidenced by the observation that the transgenic plants were larger than the control under cycling drought conditions, the presence of the flavodoxin protein in the plastid resulted in improved photosynthetic efficiency and reduced detrimental effects due to the loss of water.

### Q. PsaF

The PsaF gene SLL0819 (SEQ ID NO:79) was expressed in Arabidopsis using two different constructs under control of the PcUbi promoter and targeted to mitochondria, or targeted to the plastid. Table 22 sets forth biomass and health index data obtained from the Arabidopsis plants transformed with these constructs and tested under cycling drought and well watered conditions.

TABLE 22

| Assay | | | | Percent | | Valid | Positive | Negative |
|---|---|---|---|---|---|---|---|---|
| Type | Gene | Target | Trait | Change | pValue | Events | Events | Events |
| WW | sll0819 | Mito | Day 17 | −1.8 | NS | 6 | 3 | 3 |
| WW | sll0819 | Mito | Day 21 | −1.0 | NS | 6 | 2 | 4 |
| WW | sll0819 | Mito | Health Index | 0.1 | NS | 6 | 3 | 3 |
| WW | sll0819 | Chlor | Day 17 | 22.4 | 0.00 | 5 | 5 | 0 |
| WW | sll0819 | Chlor | Day 21 | 21.2 | 0.00 | 5 | 5 | 0 |
| WW | sll0819 | Chlor | Health Index | −0.3 | NS | 5 | 1 | 4 |

Table 22 shows that transgenic plants expressing the ssr0390 gene with targeting to the plastid were significantly larger under well-watered conditions than the control plants that did not express the sll0819 gene. In these experiments, the majority of the independent transgenic events with plastid targeting were larger than the controls in the cycling drought environment. As evidenced by the observation that the transgenic plants were larger than the control under cycling drought conditions, the presence of the PsaK protein in the plastid resulted in improved photosynthetic efficiency and reduced detrimental effects due to the loss of water.

### R. PetJ

The PetJ gene SLL1796 (SEQ ID NO:89) was expressed in Arabidopsis using two different constructs under control of the PcUbi promoter and targeted to mitochondria, or targeted to the plastid. Table 23 sets forth biomass and health index data obtained from the Arabidopsis plants transformed with these constructs and tested under cycling drought and well watered conditions.

TABLE 23

| Assay | | | | Percent | | Valid | Positive | Negative |
|---|---|---|---|---|---|---|---|---|
| Type | Gene | Target | Trait | Change | pValue | Events | Events | Events |
| CD | sll1796 | Mito | Day 20 | 12.1 | 0.001 | 7 | 6 | 1 |
| CD | sll1796 | Mito | Day 27 | 9.9 | 0.003 | 7 | 5 | 2 |
| CD | sll1796 | Mito | Health Index | 0.7 | NS | 7 | 5 | 2 |
| CD | sll1796 | Chlor | Day 20 | −20.7 | 0.000 | 4 | 0 | 4 |
| CD | sll1796 | Chlor | Day 27 | −8.1 | NS | 4 | 1 | 3 |
| CD | sll1796 | Chlor | Health Index | −9.7 | 0.016 | 4 | 0 | 4 |
| WW | sll1796 | Chlor | Day 17 | −20.5 | 0.000 | 5 | 0 | 5 |
| WW | sll1796 | Chlor | Day 21 | −15.8 | 0.000 | 5 | 0 | 5 |
| WW | sll1796 | Chlor | Health Index | 0.3 | NS | 5 | 2 | 3 |

Table 23 shows that transgenic plants expressing the sll1796 gene with targeting to the mitochondria were significantly larger under water-limited conditions than the control plants that did not express the sll1796 gene. Variation does exist among transgenic plants that contain the sll1796 gene, due to different sites of DNA insertion and other factors that impact the level or pattern of gene expression. Health Index was similar between transgenic and control plants. In these experiments, the majority of the independent transgenic events were larger than the controls.

Table 23 shows that transgenic plants expressing the sll1796 gene with subcellular targeting to the plastid were significantly smaller under water-limited and well-watered conditions than the control plants that did not express the sll11796 gene. In these experiments, all of the independent transgenic events were smaller than the controls.

As evidenced by the observation that the transgenic plants were larger than the control plants when the PetJ protein was targeted to the mitochondria, the presence of the PetJ protein in the mitochondria resulted in improved mitochondrial electron transport efficiency.

### S. PsbW

The PsbW gene SLR1739 (SEQ ID NO:91) was expressed in Arabidopsis using two different constructs under control of the PcUbi promoter and targeted to mitochondria or targeted to the plastid. Table 24 sets forth biomass and health index data obtained from the Arabidopsis plants transformed with these constructs and tested under cycling drought and well watered conditions.

TABLE 24

| Assay | | | | Percent | | Valid | Positive | Negative |
|---|---|---|---|---|---|---|---|---|
| Type | Gene | Target | Trait | Change | pValue | Events | Events | Events |
| CD | slr1739 | Mito | Day 20 | 17.1 | 0.000 | 7 | 6 | 1 |
| CD | slr1739 | Mito | Day 27 | 13.4 | 0.000 | 7 | 6 | 1 |
| CD | slr1739 | Mito | Health Index | 3.0 | NS | 7 | 5 | 2 |
| WW | slr1739 | Mito | Day 17 | 13.7 | 0.000 | 8 | 7 | 1 |
| WW | slr1739 | Mito | Day 21 | 5.6 | 0.014 | 8 | 7 | 1 |
| WW | slr1739 | Mito | Health Index | 0.7 | NS | 8 | 5 | 3 |

Table 24 shows that transgenic plants expressing the slr1739 gene were significantly larger under water-limited and well-watered conditions than the control plants that did not express the slr1739 gene. Health Index was similar between transgenic and control plants under water-limited and well-watered conditions. In these experiments, the majority of the independent transgenic events were larger than the controls in either water environment.

As evidenced by the observation that the transgenic plants were larger than the control plants when the PsbW protein was targeted to the mitochondria, the presence of the PsbW protein in the mitochondria resulted in improved electron transport efficiency in both well watered and drought conditions.

### T. CobA (Cyst)

The Uroporphyrin-III C-methyltransferase gene SLL0378 (SEQ ID NO:93) was expressed in Arabidopsis using two different constructs under control of the PcUbi promoter and targeted to mitochondria or targeted to the plastid. Table 25 sets forth biomass and health index data obtained from the Arabidopsis plants transformed with these constructs and tested under cycling drought and well watered conditions.

TABLE 25

| Assay | | | | Percent | | Valid | Positive | Negative |
|---|---|---|---|---|---|---|---|---|
| Type | Gene | Target | Trait | Change | pValue | Events | Events | Events |
| CD | sll0378 | Mito | Day 20 | −16.1 | 0.000 | 6 | 2 | 4 |
| CD | sll0378 | Mito | Day 27 | −10.6 | 0.005 | 6 | 2 | 4 |
| CD | sll0378 | Mito | Health Index | −12.6 | 0.003 | 6 | 1 | 5 |
| CD | sll0378 | Chlor | Day 20 | 8.1 | 0.041 | 5 | 3 | 2 |
| CD | sll0378 | Chlor | Day 27 | 10.4 | 0.004 | 5 | 3 | 2 |
| CD | sll0378 | Chlor | Health Index | 8.9 | 0.024 | 5 | 4 | 1 |
| WW | sll0378 | Mito | Day 17 | −15.6 | 0.001 | 6 | 2 | 4 |
| WW | sll0378 | Mito | Day 21 | −21.5 | 0.000 | 6 | 2 | 4 |
| WW | sll0378 | Mito | Health Index | 28.0 | 0.000 | 6 | 5 | 1 |
| WW | sll0378 | Chlor | Day 17 | 10.1 | 0.000 | 5 | 4 | 1 |
| WW | sll0378 | Chlor | Day 21 | 6.1 | 0.005 | 5 | 4 | 1 |
| WW | sll0378 | Chlor | Health Index | −1.4 | NS | 5 | 1 | 4 |

Table 25 shows that transgenic plants expressing the sll0378 gene with targeting to the plastid were significantly larger under water-limited and well-watered conditions than the control plants that did not express the sll0378 gene. In addition, the transgenic plants grown under water-limited conditions were darker green in color than the controls as shown by the increased health index. This suggests that the plants produced more chlorophyll or had less chlorophyll degradation during stress than the control plants.

Table 25 shows that transgenic plants expressing the sll0378 gene with targeting to the mitochondria were significantly smaller under water-limited and well-watered conditions than the control plants that did not express the sll0378 gene. Additionally, these transgenic plants had lower health index scores relative to the control in water-limited conditions, but higher health index scores in well-watered conditions. In these experiments, the majority of the independent transgenic events with plastid targeting were larger than the controls in the either environment.

As evidenced by the observation that the transgenic plants were larger than the control plants when the CobA protein was targeted to the plastid, but not when it was targeted to the mitochondria, the presence of the CobA protein in the plastid resulted in improved light harvesting capacity and more efficient energy transfer to the photosystems.

### U. Precorrin-8w Decarboxylase (CbiT, CobL)

The precorrin-8w decarboxylase gene Sll1368 (SEQ ID NO:95) was expressed Arabidopsis under control of the PcUbi promoter with no subcellular targeting. Table 26 sets forth biomass and health index data obtained from the Arabidopsis plants transformed with these constructs and tested under cycling drought and well watered conditions.

TABLE 26

| Assay | | | | Percent | | Valid | Positive | Negative |
|---|---|---|---|---|---|---|---|---|
| Type | Gene | Target | Trait | Change | pValue | Events | Events | Events |
| CD | slr1368 | None | Day 20 | 12.7 | 0.000 | 6 | 6 | 0 |
| CD | slr1368 | None | Day 27 | 7.6 | 0.017 | 6 | 5 | 1 |
| CD | slr1368 | None | Health Index | 7.7 | 0.004 | 6 | 6 | 0 |
| WW | slr1368 | None | Day 17 | 2.9 | NS | 6 | 3 | 3 |
| WW | slr1368 | None | Day 21 | 1.0 | NS | 6 | 3 | 3 |
| WW | slr1368 | None | Health Index | 3.0 | NS | 6 | 5 | 1 |

Table 26 shows that transgenic plants expressing the slr1368 gene were significantly larger under water-limited conditions than the control plants that did not express the slr1368 gene. In addition, the transgenic plants grown under water-limited conditions were darker green in color than the controls as shown by the increased health index. This suggests that the plants produced more chlorophyll or had less chlorophyll degradation during stress than the control plants. In these experiments, the majority of the independent transgenic events were larger than the controls in the water-limited environment.

Transgenic plants expressing the slr1368 gene grown under well-watered conditions were not significantly different from the controls in biomass or health index.

As evidenced by the observation that the transgenic plants were larger than the control plants, the presence of the CbiT protein resulted in improved light harvesting capacity and more efficient energy transfer to the photosystems.

V. Decarboxylating Precorrin-6y c5,15-methyltransferase (CobL, CbiE/CbiT)

The decarboxylating precorrin-6y c5, 15-methyltransferase gene Sll0099 (SEQ ID NO:97) was expressed in Arabidopsis using two different constructs under control of the PcUbi promoter and targeted to the mitochondria or with no targeting. Table 27 sets forth biomass and health index data obtained from the Arabidopsis plants transformed with these constructs and tested under cycling drought and well watered conditions.

TABLE 27

| Assay | | | | Percent | | Valid | Positive | Negative |
|---|---|---|---|---|---|---|---|---|
| Type | Gene | Target | Trait | Change | pValue | Events | Events | Events |
| WW | sll0099 | Mito | Day 17 | 11.1 | 0.000 | 6 | 5 | 1 |
| WW | sll0099 | Mito | Day 21 | 5.7 | 0.008 | 6 | 5 | 1 |
| WW | sll0099 | Mito | Health Index | 3.1 | NS | 6 | 4 | 2 |
| CD | sll0099 | Mito | Day 20 | 13.4 | 0.000 | 6 | 5 | 1 |
| CD | sll0099 | Mito | Day 27 | 2.1 | NS | 6 | 3 | 3 |
| CD | sll0099 | Mito | Health Index | 13.3 | 0.000 | 6 | 5 | 1 |
| CD | sll0099 | None | Day 20 | 23.4 | 0.000 | 7 | 7 | 0 |
| CD | sll0099 | None | Day 27 | 7.9 | 0.046 | 7 | 4 | 3 |
| CD | sll0099 | None | Health Index | 16.2 | 0.000 | 7 | 6 | 1 |

Table 27 shows that transgenic plants expressing the sll0099 gene with targeting to the mitochondria were significantly larger under water-limited and well-watered conditions than the control plants that did not express the sll0099 gene. In addition, the transgenic plants grown under water-limited conditions were darker green in color than the controls as shown by the increased health index. This suggests that the plants produced more chlorophyll or had less chlorophyll degradation during stress than the control plants. Transgenic plants expressing the sll0099 gene with no targeting were also significantly larger and had higher health index scores under water-limited conditions than the controls. In these experiments, the majority of the independent transgenic events were larger than the controls in either environment.

As evidenced by the observation that the transgenic plants were larger than the control plants, the presence of the CobL protein resulted in improved light harvesting capacity and more efficient energy transfer to the photosystems.

1. A transgenic plant transformed with an expression cassette comprising, in operative association,
a) an isolated polynucleotide encoding a promoter;
b) an isolated polynucleotide encoding a plastid transit peptide; and
c) an isolated polynucleotide encoding a polypeptide selected from the group consisting of
i) a polypeptide comprising amino acids 1 to 217 of SEQ ID NO:82;
ii) a polypeptide comprising amino acids 1 to 220 of SEQ ID NO:84;
iii) a polypeptide comprising amino acids 1 to 224 of SEQ ID NO:86;
iv) a polypeptide comprising amino acids 1 to 224 of SEQ ID NO:88;
v) a polypeptide comprising amino acids 1 to 278 of SEQ ID NO:48;
vi) a polypeptide comprising amino acids 1 to 277 of SEQ ID NO:50;
vii) a polypeptide comprising amino acids 1 to 161 of SEQ ID NO:52;
viii) a polypeptide comprising amino acids 1 to 280 of SEQ ID NO:54;
ix) a polypeptide comprising amino acids 1 to 293 of SEQ ID NO:56; and
x) a polypeptide comprising amino acids 1 to 86 of SEQ ID NO:70;
wherein the transgenic plant demonstrates increased yield as compared to a wild type plant of the same variety which does not comprise the expression cassette.

2. (canceled)

3. The transgenic plant of claim 1, further described as a species selected from the group consisting of maize, soybean, cotton, canola, rice, wheat, or sugarcane.

4. A seed which is true breeding for a transgene comprising an expression cassette comprising, in operative association,
a) an isolated polynucleotide encoding a promoter;
b) an isolated polynucleotide encoding a plastid transit peptide; and
c) an isolated polynucleotide encoding a polypeptide selected from the group consisting of:
i) a polypeptide comprising amino acids 1 to 217 of SEQ ID NO:82;
ii) a polypeptide comprising amino acids 1 to 220 of SEQ ID NO:84;
iii) a polypeptide comprising amino acids 1 to 224 of SEQ ID NO:86;
iv) a polypeptide comprising amino acids 1 to 224 of SEQ ID NO:88;
v) a polypeptide comprising amino acids 1 to 278 of SEQ ID NO:48;
vi) a polypeptide comprising amino acids 1 to 277 of SEQ ID NO:50;
vii) a polypeptide comprising amino acids 1 to 161 of SEQ ID NO:52;
viii) a polypeptide comprising amino acids 1 to 280 of SEQ ID NO:54;
ix) a polypeptide comprising amino acids 1 to 293 of SEQ ID NO:56; and
x) a polypeptide comprising amino acids 1 to 86 of SEQ ID NO:70.

5. (canceled)

6. The transgenic plant of claim 1, further described as a species selected from the group consisting of maize, soybean, cotton, canola, rice, or wheat.

7. A method of increasing yield of a plant, the method comprising the steps of
a) transforming a wild type plant cell with a transgene comprising an expression cassette comprising, in operative association,
i) n an isolated polynucleotide encoding a promoter;
ii) an isolated polynucleotide encoding a plastid transit peptide; and
iii) an isolated polynucleotide encoding a polypeptide selected from the group consisting of a polypeptide comprising amino acids 1 to 217 of SEQ ID NO:82; a polypeptide comprising amino acids 1 to 220 of SEQ ID NO:84, a tide comprising amino acids 1 to 224 of SEQ ID NO:86; a polypeptide comprising amino acids 1 to 224 of SEQ ID NO:88; a polypeptide comprising amino acids 1 to 278 of SEQ ID NO:48; a polypeptide comprising amino acids 1 to 277 of SEQ ID NO:50; a polypeptide comprising amino acids 1 to 161 of SEQ ID NO:52; a polypeptide comprising amino acids 1 to 280 of SEQ ID NO:54; a polypeptide comprising amino acids 1 to 293 of SEQ ID NO:56; and a polypeptide comprising amino acids 1 to 86 of SE ID NO:70;
b) regenerating transgenic plantlets from the transformed plant cells; and
c) selecting transgenic plants which demonstrate increased yield.

8. (canceled)

9. The method of claim 7, wherein the plant is maize, soybean, cotton, canola, rice, wheat, or sugarcane.

10. A transgenic plant transformed with an expression cassette comprising, in operative association,
a) an isolated polynucleotide encoding a promoter capable of enhancing expression in leaves; and
b) an isolated polynucleotide encoding a polypeptide selected from the group consisting of a polypeptide comprising amino acids 1 to 421 of SEQ ID NO: 2; a polypeptide comprising amino acids 1 to 174 of SEQ ID NO: 4; a polypeptide comprising amino acids 1 to 55 of SEQ ID NO: 6; a polypeptide comprising amino acids 1 to 96 of SEQ ID NO: 8; a polypeptide comprising amino acids 1 to 384 of SEQ ID NO: 20; and a polypeptide comprising amino acids 1 to 149 of SEQ ID NO:22;
wherein the transgenic plant demonstrates increased yield as compared to a wild type plant of the same variety which does not comprise the expression cassette,

11. A seed which is true breeding for a transgene comprising an expression cassette comprising, in operative association,
a) an isolated polynucleotide encoding a promoter capable of enhancing expression in leaves; and
b) an isolated polynucleotide encoding a polypeptide selected from the group consisting of a polypeptide comprising amino acids 1 to 421 of SEQ ID NO: 2; a polypeptide comprising amino acids 1 to 174 of SEQ ID NO: 4; a polypeptide comprising amino acids 1 to 55 of SEQ ID NO: 6; a polypeptide comprising amino acids 1 to 96 of SEQ ID NO: 8; a polypeptide comprising amino acids 1 to 384 of SEQ ID NO: 20; and a polypeptide comprising amino acids 1 to 149 of SEQ ID NO:22.

12. A method of increasing yield of a plant, the method comprising the steps of
a) transforming a wild type plant cell with a transgene comprising an expression cassette comprising, in operative association,
i) an isolated polynucleotide encoding a promoter capable of enhancing expression in leaves; and
ii) an isolated polynucleotide encoding a polypeptide selected from the group consisting of a polypeptide comprising amino acids 1 to 421 of SEQ ID NO: 2; a polypeptide comprising amino acids 1 to 174 of SEQ ID NO: 4; a polypeptide comprising amino acids 1 to 55 of SEQ ID NO: 6; a polypeptide comprising amino acids 1 to 96 of SEQ ID NO: 8; a polypeptide comprising amino acids 1 to 384 of SEQ ID NO: 20; and a polypeptide comprising amino acids 1 to 149 of SEQ ID NO:22;
b) regenerating transgenic plantlets from the transformed plant cells; and
c) selecting transgenic plants which demonstrate increased yield.

13. A transgenic plant transformed with an expression cassette comprising, in operative association,
a) an isolated polynucleotide encoding a promoter capable of enhancing expression in leaves;
b) an isolated polynucleotide encoding a chloroplast transit peptide; and
c) an isolated polynucleotide encoding a polypeptide selected from the group consisting of a polypeptide comprising amino acids 1 to 128 of SEQ ID NO: 10 and a polypeptide comprising amino acids 1 to 115 of SEQ ID NO:12;
wherein the transgenic plant demonstrates increased yield as compared to a wild type plant of the same variety which does not comprise the expression cassette.

14. A seed which is true breeding for a transgene comprising an expression cassette comprising, in operative association,
a) an isolated polynucleotide encoding a promoter capable of enhancing expression in leaves;
b) an isolated polynucleotide encoding a chloroplast transit peptide; and
c) an isolated polynucleotide encoding a polypeptide selected from the group consisting of a polypeptide comprising amino acids 1 to 128 of SEQ ID NO: 10 and a polypeptide comprising amino acids 1 to 115 of SEQ ID NO:12.

15. A method of increasing yield of a plant, the method comprising the steps of
a) transforming a wild type plant cell with a transgene comprising an expression cassette comprising, in operative association,
i) an isolated polynucleotide encoding a promoter capable of enhancing expression in leaves;
ii) an isolated polynucleotide encoding a chloroplast transit peptide; and
iii) an isolated polynucleotide encoding a polypeptide selected from the group consisting of a polypeptide comprising amino acids 1 to 128 of SEQ ID NO: 10 and a polypeptide comprising amino acids 1 to 115 of SEQ ID NO:12;
b) regenerating transgenic plantlets from the transformed plant cells; and
c) selecting transgenic plants which demonstrate increased yield.

16. A transgenic plant transformed with an expression cassette comprising, in operative association,
a) an isolated polynucleotide encoding a promoter capable of enhancing expression in leaves;
b) an isolated polynucleotide encoding a mitochondrial transit peptide; and
c) an isolated polynucleotide encoding a polypeptide selected from the group consisting of a polypeptide comprising amino acids 1 to 569 of SEQ ID NO: 26; a polypeptide comprising amino acids 1 to 565 of SEQ ID NO: 28; a polypeptide comprising amino acids 1 to 551 of SEQ ID NO:30; a polypeptide comprising amino acids 1 to 331 of SEQ ID NO: 32; a polypeptide comprising amino acids 1 to 335 of SEQ ID NO: 34; a polypeptide comprising amino acids 1 to 332 of SEQ ID NO:36; a polypeptide comprising amino acids 1 to 238 of SEQ ID NO: 38; a polypeptide comprising amino acids 1 to 106 of SEQ ID NO: 42; a polypeptide comprising amino acids 1 to 105 of SEQ ID NO:44; and a polypeptide comprising amino acids 1 to 450 of SEQ ID NO:60; wherein the transgenic plant demonstrates increased yield as compared to a wild type plant of the same variety which does not comprise the expression cassette.

17. A seed which is true breeding for a transgene comprising an expression cassette comprising, in operative association,
a) an isolated polynucleotide encoding a promoter capable of enhancing expression in leaves;
b) an isolated polynucleotide encoding a mitochondrial transit peptide; and
c) an isolated polynucleotide encoding a polypeptide selected from the group consisting of a polypeptide comprising amino acids 1 to 569 of SEQ ID NO: 26; a polypeptide comprising amino acids 1 to 565 of SEQ ID NO: 28; a polypeptide comprising amino acids 1 to 551 of SEQ ID NO:30; a polypeptide comprising amino acids 1 to 331 of SEQ ID NO: 32; a polypeptide comprising amino acids 1 to 335 of SEQ ID NO: 34; a polypeptide comprising amino acids 1 to 332 of SEQ ID NO:36; a polypeptide comprising amino acids 1 to 238 of SEQ ID NO: 38; a polypeptide comprising amino acids 1 to 106 of SEQ ID NO: 42; a polypeptide comprising amino acids 1 to 105 of SEQ ID NO:44; and a polypeptide comprising amino acids 1 to 450 of SEQ ID NO:60.

18. A method of increasing yield of a plant, the method comprising the steps of
a) transforming a wild type plant cell with a transgene comprising an expression cassette comprising, in operative association,
i) an isolated polynucleotide encoding a promoter capable of enhancing expression in leaves;
ii) an isolated polynucleotide encoding a mitochondrial transit peptide; and
iii) an isolated polynucleotide encoding a polypeptide selected from the group consisting of a polypeptide comprising amino acids 1 to 569 of SEQ ID NO: 26; a polypeptide comprising amino acids 1 to 565 of SEQ ID NO: 28; a polypeptide comprising amino acids 1 to 551 of SEQ ID NO:30; a polypeptide comprising amino acids 1 to 331 of SEQ ID NO: 32; a polypeptide comprising amino acids 1 to 335 of SEQ ID NO: 34; a polypeptide comprising amino acids 1 to 332 of SEQ ID NO:36; a polypeptide comprising amino acids 1 to 238 of SEQ ID NO: 38; a polypeptide comprising amino acids 1 to 106 of SEQ ID NO: 42; a polypeptide comprising amino acids 1 to 105 of SEQ ID NO:44; and a polypeptide comprising amino acids 1 to 450 of SEQ ID NO:60;
b) regenerating transgenic plantlets from the transformed plant cells; and
c) selecting transgenic plants which demonstrate increased yield.

19. A transgenic plant transformed with an expression cassette comprising, in operative association,
a) an isolated polynucleotide encoding a promoter;
b) an isolated polynucleotide encoding a mitochondrial transit peptide; and
c) an isolated polynucleotide encoding a polypeptide selected from the group consisting of a polypeptide comprising amino acids 1 to 388 of SEQ ID NO: 64; a polypeptide comprising amino acids 1 to 276 of SEQ ID NO: 68; a polypeptide comprising amino acids 1 to 122 of SEQ ID NO:72; a polypeptide comprising amino acids 1 to 128 of SEQ ID NO: 74; a polypeptide comprising amino acids 1 to 179 of SEQ ID NO: 76; and a polypeptide comprising amino acids 1 to 122 of SEQ ID NO: 92;
wherein the transgenic plant demonstrates increased yield as compared to a wild type plant of the same variety which does not comprise the expression cassette.

20. A seed which is true breeding for a transgene comprising an expression cassette comprising, in operative association,
a) an isolated polynucleotide encoding a promoter;
b) an isolated polynucleotide encoding a mitochondrial transit peptide; and
c) an isolated polynucleotide encoding a polypeptide selected from the group consisting of a polypeptide comprising amino acids 1 to 388 of SEQ ID NO: 64; a polypeptide comprising amino acids 1 to 276 of SEQ ID NO: 68; a polypeptide comprising amino acids 1 to 122 of SEQ ID NO:72; a polypeptide comprising amino acids 1 to 128 of SEQ ID NO: 74; a polypeptide comprising amino acids 1 to 179 of SEQ ID NO: 76; and a polypeptide comprising amino acids 1 to 122 of SEQ ID NO: 92.

21. A method of increasing yield of a plant, the method comprising the steps of
a) transforming a wild type plant cell with a transgene comprising an expression cassette comprising, in operative association,
i) an isolated polynucleotide encoding a promoter;
ii) an isolated polynucleotide encoding a mitochondrial transit peptide; and
iii) an isolated polynucleotide encoding a polypeptide selected from the group consisting of a polypeptide comprising amino acids 1 to 388 of SEQ ID NO: 64; a polypeptide comprising amino acids 1 to 276 of SEQ ID NO: 68; a polypeptide comprising amino acids 1 to 122 of SEQ ID NO:72; a polypeptide comprising amino acids 1 to 128 of SEQ ID NO: 74; a polypeptide comprising amino acids 1 to 179 of SEQ ID NO: 76; and a polypeptide comprising amino acids 1 to 122 of SEQ ID NO: 92;
b) regenerating transgenic plantlets from the transformed plant cells; and
c) selecting transgenic plants which demonstrate increased yield.

22. A transgenic plant transformed with an expression cassette comprising, in operative association,
a) an isolated polynucleotide encoding a promoter; and
b) an isolated polynucleotide encoding a polypeptide selected from the group consisting of a polypeptide comprising amino acids 1 to 197 of SEQ ID NO: 96;
wherein the transgenic plant demonstrates increased yield as compared to a wild type plant of the same variety which does not comprise the expression cassette,

23. A seed which is true breeding for a transgene comprising an expression cassette comprising, in operative association,
a) an isolated polynucleotide encoding a promoter; and
b) an isolated polynucleotide encoding a polypeptide selected from the group consisting of a polypeptide comprising amino acids 1 to 197 of SEQ ID NO: 96.

24. A method of increasing yield of a plant, the method comprising the steps of
a) transforming a wild type plant cell with a transgene comprising an expression cassette comprising, in operative association,
i) an isolated polynucleotide encoding a promoter; and
ii) an isolated polynucleotide encoding a polypeptide selected from the group consisting of a polypeptide comprising amino acids 1 to 197 of SEQ ID NO: 96;
b) regenerating transgenic plantlets from the transformed plant cells; and
c) selecting transgenic plants which demonstrate increased yield.

25. An isolated polynucleotide encoding a polypeptide selected from the group consisting of a polypeptide comprising amino acids 1 to 384 of SEQ ID NO: 20; a polypeptide comprising amino acids 1 to 569 of SEQ ID NO: 26; a polypeptide comprising amino acids 1 to 565 of SEQ ID NO: 28; a polypeptide comprising amino acids 1 to 551 of SEQ ID NO:30; a polypeptide comprising amino acids 1 to 331 of SEQ ID NO: 32; a polypeptide comprising amino acids 1 to 335 of SEQ ID NO: 34; a polypeptide comprising amino acids 1 to 332 of SEQ ID NO:36; a polypeptide comprising amino acids 1 to 238 of SEQ ID NO: 38; a polypeptide comprising amino acids 1 to 106 of SEQ ID NO: 42; a polypeptide comprising amino acids 1 to 105 of SEQ ID NO:44; a polypeptide comprising amino acids 1 to 278 of SEQ ID NO:48; a polypeptide comprising amino acids 1 to 277 of SEQ ID NO:50; a polypeptide comprising amino acids 1 to 161 of SEQ ID NO:52; a polypeptide comprising amino acids 1 to 280 of SEQ ID NO:54; a polypeptide comprising amino acids 1 to 293 of SEQ ID NO:56; a polypeptide comprising amino acids 1 to 450 of SEQ ID NO:60; a polypeptide comprising amino acids 1 to 388 of SEQ ID NO:64; a polypeptide comprising amino acids 1 to 128 of SEQ ID NO:74; a polypeptide comprising amino acids 1 to 179 of SEQ ID NO:76; a polypeptide comprising amino acids 1 to 217 of SEQ ID NO:82; a polypeptide comprising amino acids 1 to 220 of SEQ ID NO:84; a polypeptide comprising amino acids 1 to 224 of SEQ ID NO:86; and a polypeptide comprising amino acids 1 to 224 of SEQ ID NO:88.
